(19) United States
(12) Patent Application Publication
(10) Pub. No.: US 2022/0267445 A1
(43) Pub. Date: Aug. 25, 2022

(54) Methods and Compositions for Determining Susceptibility to Treatment with Checkpoint Inhibitors

(71) Applicant: Novocure GmbH, Root D4 (CH)

(72) Inventors: David TRAN, Gainesville, FL (US); Dongjiang Chen, Gainesville, FL (US)

(73) Assignee: Novocure GmbH, Root D4 (CH)

(21) Appl. No.: 17/673,602

(22) Filed: Feb. 16, 2022

Related U.S. Application Data
(60) Provisional application No. 63/172,862, filed on Apr. 9, 2021.
(60) Provisional application No. 63/150,359, filed on Feb. 17, 2021.

(51) Int. Cl. C07K 16/28; A61P 35/00

(52) U.S. Cl. CPC C07K 16/2818; A61K 2039/505; A61P 35/00

(57) ABSTRACT

Methods and kits treating a subject with a checkpoint inhibitor are provided by detecting expression levels of one or more biomarkers (e.g., cytokines and cytotoxic genes, immune cell functional regulators, naïve immune cell markers, regulatory T cell factors, and immune inhibitory receptors) in immune cells of a patient or subject with a condition (e.g., cancer) before and after exposure of tumor cells to alternating electric fields. Kits comprising nucleic acid probes for detecting the one or more biomarkers are also provided.

### CROSS REFERENCE TO RELATED APPLICATIONS

This application claims the benefit of U.S. Provisional Applications 63/150,359, filed Feb. 17, 2021, and 63/172,862, filed Apr. 9, 2021, both of which are incorporated herein by reference in their entirety.

All references cited herein, including but not limited to patents and patent applications, are incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 30, 2022, is named 1459-0091US01_SL.txt and is 10,935 bytes in size.

### BACKGROUND

Tumor Treating Fields (TTFields) are an effective anti-neoplastic treatment that involves applying low intensity, intermediate frequency (e.g., 50 kHz-1 MHz or 100-500 kHz), alternating electric fields to a target region.

In the in vivo context, TTFields therapy can be delivered using a wearable and portable device (Optune®). The delivery system includes an electric field generator, four adhesive patches (non-invasive, insulated transducer arrays), rechargeable batteries and a carrying case. The transducer arrays are applied to the skin and are connected to the device and battery. The therapy is designed to be worn for as many hours as possible throughout the day and night. In the preclinical setting, TTFields can be applied in vitro using, for example, the Inovitro™ TTFields lab bench system. Inovitro™ includes a TTFields generator and base plate containing 8 ceramic dishes per plate. Cells are plated on cover slips placed inside each dish. TTFields are applied using two perpendicular pairs of transducer arrays insulated by a high dielectric constant ceramic in each dish. In both the in vivo and in vitro contexts, the orientation of the TTFields is switched 90° every 1 second, thus covering different orientation axes of cell divisions.

GBM, the most common and lethal brain cancer in adults (1, 2), is also one of the least immunogenic tumors. Recent work has collectively demonstrated striking immune dysregulation and functional impairment in patients with GBM. The tumor immune microenvironment (TiME) in GBM is profoundly immunosuppressed, characterized by higher expression of immune checkpoint proteins and infiltration of immune suppressive cells, lower numbers of tumor infiltrating lymphocytes, systemic T cell lymphopenia and anergy, cytokine dysregulation among others (3,4). In addition, the blood brain barrier further diminishes exposure of tumor-associated antigens to immune cells and vice versa, further hindering immunotherapeutic efforts (4).

A gene signature is a gene or group of genes that have a characteristic expression pattern as a result of a biological process, disease or condition, or response to a treatment or other external event. For example, one or more genes in a gene signature can have increased or decreased expression levels after a patient or subject is exposed to a treatment or environmental condition. The collective pattern of altered expression levels as a whole can serve as a marker to determine the presence or absence of biological conditions prior to or after treatment for a disease or condition or to select and/or predict those patients or subjects that have a higher or lower chance of responding to the said treatment or subsequent treatment or that have a higher or lower chance of worsening of the disease or condition.

### SUMMARY

As described herein, TTFields can be applied to tumor cells of a subject in order to activate the immune system. The activation of the immune system by TTFields can be assessed by measuring the expression level (e.g., mRNA, other nucleic acids, or protein expression level) of one or more genes comprising a gene signature. The pattern of expression of the genes of the gene signature can then be used to determine if the subject is susceptible to treatment of the tumor with, for example, checkpoint inhibitors.

One aspect provides a method of treating a subject with a checkpoint inhibitor by (a) determining a first expression level of nucleic acids expressing cytokines and cytotoxic genes in immune T cells of the subject; (b) determining a first expression level of nucleic acids expressing T cell functional regulators in immune T cells of the subject; (c) determining a first expression level of nucleic acids expressing naïve T cell markers in immune T cells of the subject; (d) determining a first expression level of nucleic acids expressing regulatory T cell factors in immune T cells of the subject; (e) determining a first expression level of nucleic acids expressing immune inhibitory receptors in immune T cells of the subject; and (f) determining a first expression level of nucleic acids expressing type 1 interferon response genes in immune T cells of the subject.

Alternating electric fields can be applied to tumor cells of the subject at a frequency between 50 kHz-1 MHz, preferably between 100 and 500 kHz, after determining the first expression level (e.g., steps a-f above) and prior to determining the second expression level (e.g., steps h-m below).

The method further includes (h) determining a second expression level of nucleic acids expressing cytokines and cytotoxic genes in immune T cells of the subject; (i) determining a second expression level of nucleic acids expressing T cell functional regulators in immune T cells of the subject; (j) determining a second expression level of nucleic acids expressing naïve T cell markers in immune T cells of the subject; (k) determining a second expression level of nucleic acids expressing regulatory T cell factors in immune T cells of the subject; (1) determining a second expression level of nucleic acids expressing immune inhibitory receptors in immune T cells of the subject; and (m) determining a second expression level of nucleic acids expressing type 1 interferon response genes in immune T cells of the subject.

The subject is treated with a checkpoint inhibitor if (i) the first expression level of at least 50% of the nucleic acids expressing cytokines and cytotoxic genes is lower than the second expression level of nucleic acids expressing cytokines and cytotoxic genes, (ii) the first expression level of at least 50% of the nucleic acids expressing T cell functional regulators is lower than the second expression level of nucleic acids expressing T cell functional regulators, (iii) the first expression level of at least 50% of the nucleic acids expressing naïve T cell markers is greater than the second expression level of nucleic acids expressing naïve T cell markers, (iv) the first expression level of at least 50% of the nucleic acids expressing regulatory T cell factors is greater than the second expression level of nucleic acids expressing regulatory T cell factors, (v) the first expression level of at least 50% of the nucleic acids expressing immune inhibitory receptors is either greater than or unchanged compared to the second expression level of nucleic acids expressing immune inhibitory receptors, and (vi) the first expression level of nucleic acids expressing type 1 interferon response genes is either greater or lower than or unchanged compared to the second expression level of nucleic acids expressing type 1 interferon response genes.

Another aspect described herein provides a method including steps of (a) determining in immune cells of a subject a first expression level of the following biomarker(s): cytokines and cytotoxic genes, immune cell functional regulators, naïve immune cell markers, regulatory T cell factors, or immune inhibitory receptors, or combinations thereof; (b) applying alternating electric fields to tumor cells of the subject at a frequency between 50 kHz-1 MHz, preferably between 100 and 500 kHz, after step (a) and prior to step (c); and (c) determining in immune cells of the subject a second expression level of the biomarker(s) of step (a).

Optionally, step (a) comprises determining a first expression level of cytokines and cytotoxic genes, or step (a) comprises determining a first expression level of immune cell functional regulators, or step (a) comprises both determining a first expression level of cytokines and cytotoxic genes and determining a first expression level of immune cell functional regulators.

In an aspect the immune cell functional regulators are T cell functional regulators or natural killer cells.

In one aspect, step (a) includes determining a first expression level of cytokines and cytotoxic genes, immune cell functional regulators, naïve immune cell markers, regulatory T cell factors, and immune inhibitory receptors.

Biomarker expression levels may be determined by nucleic acid expression or by expression of a corresponding protein.

In another aspect, the method may subsequently include treating the subject with a checkpoint inhibitor if (i) the first expression level of at least 50% of the cytokines and cytotoxic genes is lower than the second expression level of cytokines and cytotoxic genes, (ii) the first expression level of at least 50% of the immune cell functional regulators is lower than the second expression level of immune cell functional regulators, (iii) the first expression level of at least 50% of the naïve immune cell markers is greater than the second expression level of naïve immune cell markers, (iv) the first expression level of at least 50% of the regulatory T cell factors is greater than the second expression level of regulatory T cell factors, or (v) the first expression level of at least 50% of the immune inhibitory receptors is either greater than or unchanged compared to the second expression level of immune inhibitory receptors. This aspect may further include treating the subject with a checkpoint inhibitor if the first expression level of at least 50% of the cytokines and cytotoxic genes is lower than the second expression level of cytokines and cytotoxic genes; or treating the subject with a checkpoint inhibitor if the first expression level of at least 50% of the immune cell functional regulators is lower than the second expression level of immune cell functional regulators; or treating the subject with a checkpoint inhibitor if both (i) the first expression level of at least 50% of the cytokines and cytotoxic genes is lower than the second expression level of cytokines and cytotoxic genes, and (ii) the first expression level of at least 50% of the immune cell functional regulators is lower than the second expression level of immune cell functional regulators.

In another aspect the checkpoint inhibitor is ipilimumab pembrolizumab, nivolumab, cemilimab, atezolimumab, avelumab, durvalumab, IDO1 inhibitors, TIGIT inhibitors, LAG-3 inhibitors, TIM-3 inhibitors, VISTA inhibitors, or B7-H3 inhibitors, and the checkpoint inhibitor is for use in treatment of a subject, wherein the subject has undergone the steps of determining the first and second expression level as described above.

Another aspect provides a method of indicating the activation of a subject's immune system prior to administration of an anti-cancer drug, the method including determining, in the immune cells of the subject, first and second expression levels of the one or more biomarkers as described herein, wherein (as also described herein) the alternating electric field has been applied to tumor cells of the subject between the two determinations, and comparing the first and second expression level of the one or more biomarkers, wherein a difference in the first and second expression levels indicates the activation of the subject's immune system.

In an aspect the immune cell functional regulators are T cell functional regulators or the naïve immune cell markers are naïve T cell markers.

In an aspect the nucleic acids expressing cytokines and cytotoxic gene are GZMB, GZMH, GZMK, GNLY, PRF1, INFG, NKG7, CX3CR1, CCL3, or CCL4, or combinations thereof; or the nucleic acids expressing immune cell functional regulators are ZEB2, ZHF683, HOPX, TBX21, ID2, TOX, GF11, EOMES, or HMGB3, or combinations thereof; or the nucleic acids expressing naïve immune cell markers are TCF7, SELL, LEF1, CCR7, or IL7R, or combinations thereof; or the nucleic acids expressing naïve immune cell markers are TCF7, SELL, LEF1, CCR7, or IL7R, or combinations thereof; or the nucleic acids expressing regulatory immune cell factors are IL2RA, FOXP3, or IKZF2, or combinations thereof; or the nucleic acids expressing immune inhibitory receptors are LAG3, TIGIT, PDCD1, or CTLA4, or combinations thereof.

In yet another aspect, the nucleic acids are GZMB, GZMH, GZMK, GNLY, PRF1, INFG, NKG7, CX3CR1, CCL3, CCL4, ZEB2, ZHF683, HOPX, TBX21, ID2, TOX, GF11, EOMES, HMGB3, TCF7, SELL, LEF1, CCR7, IL7R, IL2RA, FOXP3, IKZF2, LAG3, TIGIT, PDCD1, CTLA4, or combinations thereof.

In yet another aspect, the checkpoint inhibitor is ipilimumab, pembrolizumab, nivolumab, cemilimab, atezolimumab, avelumab, durvalumab, IDO1 inhibitors, TIGIT inhibitors, LAG-3 inhibitors, TIM-3 inhibitors, VISTA inhibitors, or B7-H3 inhibitors.

The tumor cells may be brain cells, blood cells, breast cells, pancreatic cells, ovarian cells, lung cells, or mesenchymal cells. In particular, the tumor cells may be brain cells. Preferably, the tumor cells are cancer cells.

Another aspect described herein provides a kit containing nucleic acid probes for detecting nucleic acids expressing cytokines and cytotoxic genes, nucleic acids expressing T cell functional regulators, nucleic acids expressing naïve T cell markers, nucleic acids expressing regulatory T cell factors, nucleic acids expressing immune inhibitory receptors, and/or nucleic acids expressing type 1 interferon response genes.

In another aspect, the kit includes two or more, preferably three or more, more preferably four or more, nucleic acids (including probes or primers) for detecting expression of cytokines and cytotoxic genes, nucleic acids expressing T cell functional regulators, nucleic acids expressing naïve T cell markers, nucleic acids expressing regulatory T cell factors, and nucleic acids expressing immune inhibitory receptors.

In an aspect, the kit may include nucleic acids for detecting GZMB, GZMH, GZMK, GNLY, PRF1, INFG, NKG7, CX3CR1, CCL3, or CCL4; or the kit may include nucleic acids for detecting ZEB2, ZHF683, HOPX, TBX21, ID2, TOX, GF11, EOMES, or HMGB3; or the kit may include nucleic acids for detecting TCF7, SELL, LEF1, CCR7, or IL7R; or the kit may include nucleic acids for detecting IL2RA, FOXP3, or IKZF2; or the kit may include nucleic acids for detecting LAG3, TIGIT, PDCD1, or CTLA4; or the kit may include nucleic acids for detecting GZMB, GZMH, GZMK, GNLY, PRF1, INFG, NKG7, CX3CR1, CCL3, CCL4, ZEB2, ZHF683, HOPX, TBX21, ID2, TOX, GF11, EOMES, HMGB3, TCF7, SELL, LEF1, CCR7, IL7R, IL2RA, FOXP3, IKZF2, LAG3, TIGIT, PDCD1, or CTLA4; or any combinations thereof.

### BRIEF DESCRIPTION OF THE DRAWINGS

FIGS. 1A-1h show TTFields-induced cytosolic micronuclei clusters recruit cGAS and AIM2 and require G1-S entry. FIGS. 1A-1B show exemplary confocal images stained for cGAS and AIM2, b-actin for cytoplasmic outline, and DAPI for nuclear counter-staining in LN428 GBM cells non-treated (NT) with TTFields (TTF);

FIG. 1B shows the experiment of FIG. 1A with cells treated with TTFields for 24 hours with a side view (far right panel) showing that TTFields-induced cytosolic micronuclei clusters protrude directly from the true nuclei through a narrow bridge;

FIG. 1C, FIG. 1D, FIG. 1E, and FIG. 1F show exemplary confocal images with z stack showing immunofluorescent staining for cGAS, AIM2, and LAMINA/C with DAPI counter-staining in LN428 GBM cells that were pretreated with either the vehicle (FIG. 1C, FIG. 1E) or ribociclib (4.5 μM) (FIG. 1D, FIG. 1F) to induce G1 arrest, then non-treated (FIG. 1C, FIG. 1D) or treated with TTFields for 24 hours (FIG. 1E,

FIG. 1F), demonstrating that S phase entry is required for TTFields-induced cytosolic micronuclei clusters;

FIG. 1G provides an exemplary bar graph showing percentages of cells with large cytosolic micronuclei clusters with cGAS and AIM2 recruitment being dependent on TTFields in the 3 indicated GBM cell lines treated as described with respect to FIG. 1C, FIG. 1D, FIG. 1E, and FIG. 1F;

FIG. 1H provides exemplary histograms showing DNA content analysis by propidium iodide (PI) staining of LN428 cells treated with ribociclib (4.5 μM) for 0 and 24 hours demonstrating effective G₁-S arrest;

FIGS. 2A-2G show that TTFields activate the cGAS-STING inflammasome. FIG. 2A and FIG. 2B show that the cGAS-STING inflammasome's components IRF3 and p65 were activated following TTFields treatment, as determined by immunoblotting for p-IRF3 and p-p65 in total lysate (FIG. 2A) and quantified by densitometry for phospho-IRF3 (p-IRF3) and p-p65 fractions relative to total IRF3 and p65 levels, normalized against b-actin loading control with values for the non-treated condition set at 1 and (FIG. 2B) in the 3 indicated GBM cell lines either non-treated or treated with TTFields for 24 hours;

FIG. 2C shows increased concentration and recruitment of p-IRF3 and p65 in large cytosolic micronuclei clusters detected by immunofluorescent staining and confocal microscopy in LN428 cells after 24 hour treatment with TTFields;

FIG. 2D and FIG. 2E provide bar graphs demonstrating relative mRNA upregulation of several PIC genes (FIG. 2D) and T1IFNs and T1IRGs (FIG. 2E) in the 3 indicated GBM cell lines in response to 24 hour treatment with TTFields;

FIG. 2F and FIG. 2G provide exemplary bar graphs showing that TTFields-induced upregulation of PICs and T1IRGs was dependent on STING as measured in mRNA expression (FIG. 2F), and in INFb protein level in total lysate by ELISA (FIG. 2G) in the 3 indicated GBM cell lines that express a scrambled (Sc) or STING (ST KD) shRNA, and that are either non-treated or treated with TTFields for 24 hours;

FIGS. 3A-3C show TTFields activate the AIM2-caspase-1 inflammasome. FIG. 3A provides exemplary histograms of caspase-1 activation level, as determined using the fluorescently labeled specific irreversible inhibitor of activated caspase-1 FAM-YVAD-FMK (SEQ ID NO: 47), in the 3 indicated GBM cell lines that expressed a scrambled (Sc) or AIM2 (AIM2 KD) shRNA, and that were either non-treated or treated with TTFields for 24 hours;

FIG. 3B provides exemplary radiographs showing immunoblotting for GSDMD revealing the caspase-1 cleaved product (N-GSDMD) in total lysates from U87MG and LN827 cells that expressed a scrambled (Sc) or AIM2 (AIM2 KD) shRNA and were either non-treated or treated with TTFields for 24 hours;

FIG. 3C provides an exemplary bar graph showing TTFields induced increased plasma membrane disruption in a AIM2-dependent manner as determined by LDH release into the supernatants in the 3 indicated GBM cell lines that expressed a scrambled (Sc) or AIM2 (AIM2 KD) shRNA, and that were either non-treated or treated with TTFields for 24 hours;

FIGS. 4A-4L show induction of anti-tumor immunity in GBM by TTFields requires STING and AIM2. FIG. 4A provides an exemplary diagram detailing the immunization, rechallenge and monitoring schema testing the use of TTFields-treated KR158-luc murine GBM cells as a complete vaccination platform, providing both tumor-associated antigens (neoantigens) and adjuvant “danger” signal through the cGAS-STING and AIM2-caspase-1 inflammasomes;

FIG. 4B, FIG. 4C, and FIG. 4D show exemplary orthotopic KR158-luc GBM growth after immunization using KR158-luc cells with or without STING and AIM2 DKD that were non-treated or pretreated with TTFields for 72 hours, as determined by serial in vivo BLI up to 40 days after intracranial immunization (FIG. 4B), and up to 21 days post rechallenge with 2× parental KR158-luc cells (FIG. 4C), and the numbers of tumor-free animals at day 100 in each protocol as summarized in (FIG. 4D);

FIG. 4E provides an exemplary Kaplan-Meier estimate showing survival rates of animals immunized and rechallenged with KR158-luc cells in the various conditions used in FIG. 4B, FIG. 4C, and FIG. 4D;

FIG. 4F and FIG. 4G provide exemplary combo box and whisker and dot plots showing immunophenotyping of animals immunized with KR158-luc in the various conditions used in FIG. 4B, FIG. 4C, FIG. 4D, and FIG. 4E for total DCs (MHCII⁺, CD11c⁺) and the fractions of activated DCs (CD80⁺, CD86⁺) in draining deep cervical lymph nodes (dcLNs) (FIG. 4F) and for total DCs, activated DCs, and early activated CD69⁺, CD4⁺ and CD8⁺ T cells in the spleen at 2 weeks post primary immunization (FIG. 4G);

FIG. 18A, FIG. 18B, FIG. 18C, FIG. 18D, FIG. 18E, FIG. 18F, and FIG. 18G provide more detailed immunophenotyping of these same animals described in reference to FIGS. 4A-4L with respect to dcLNs, PMBCs and the spleen;

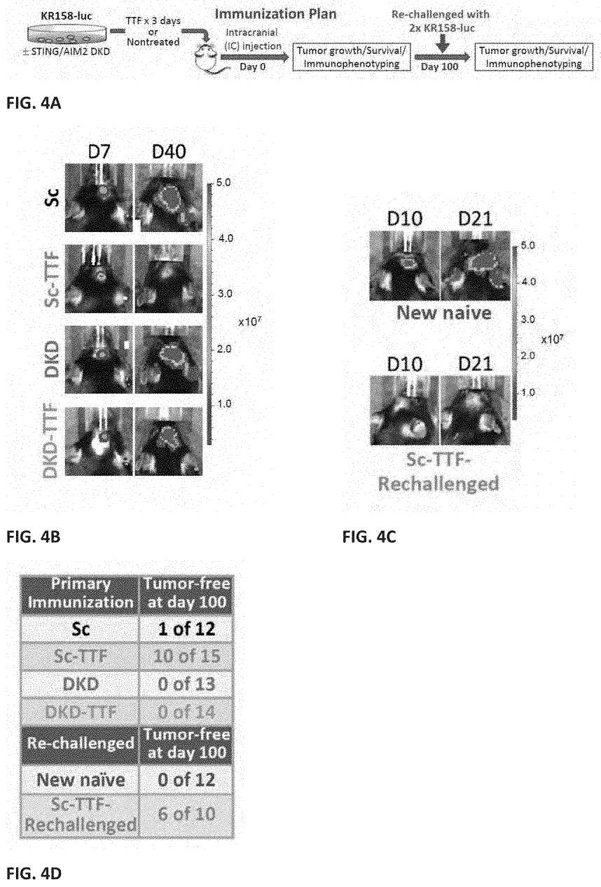

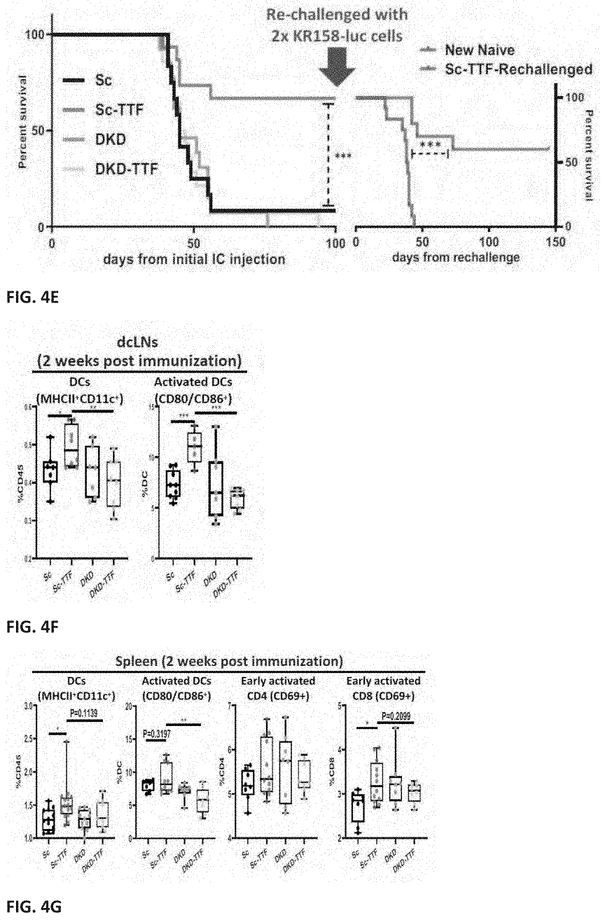

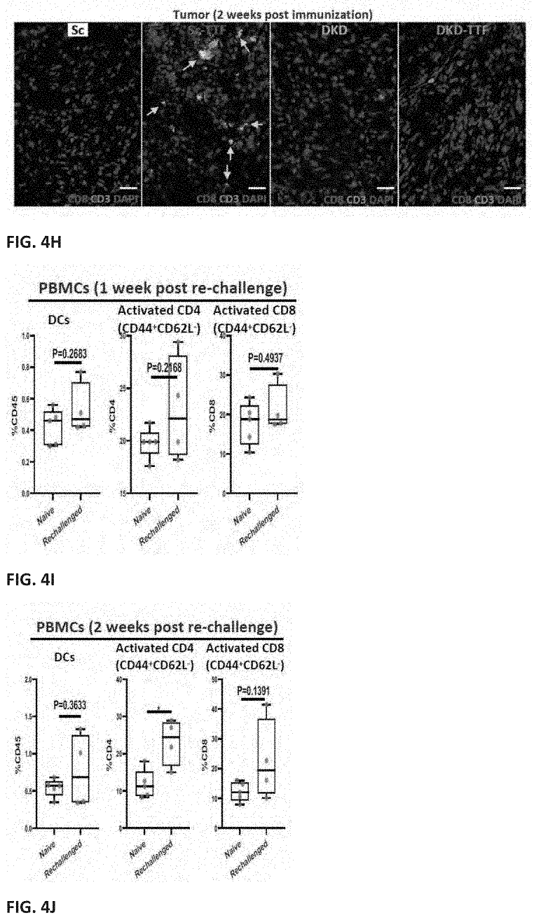

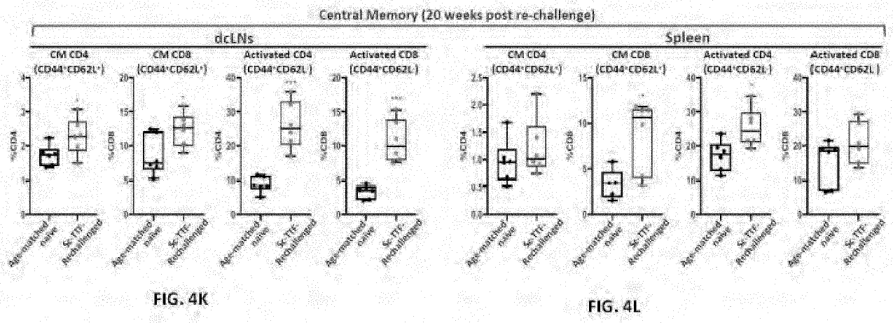

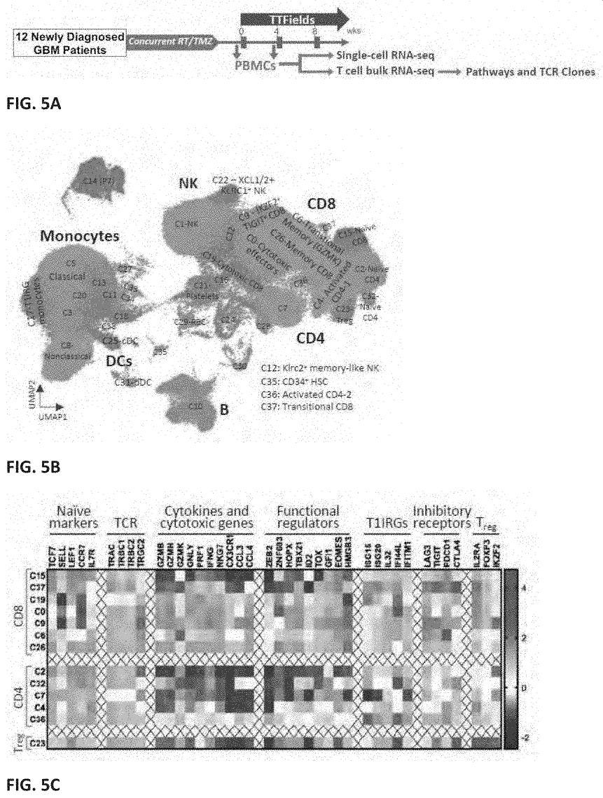

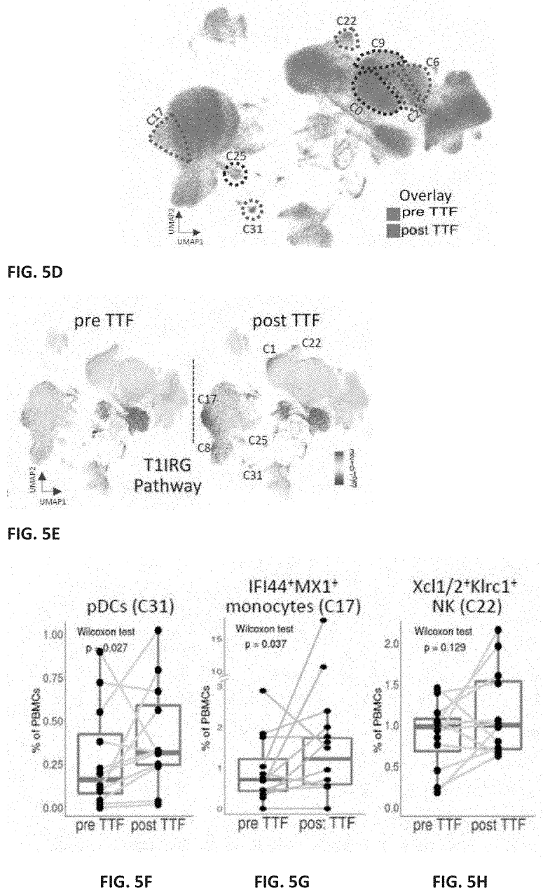

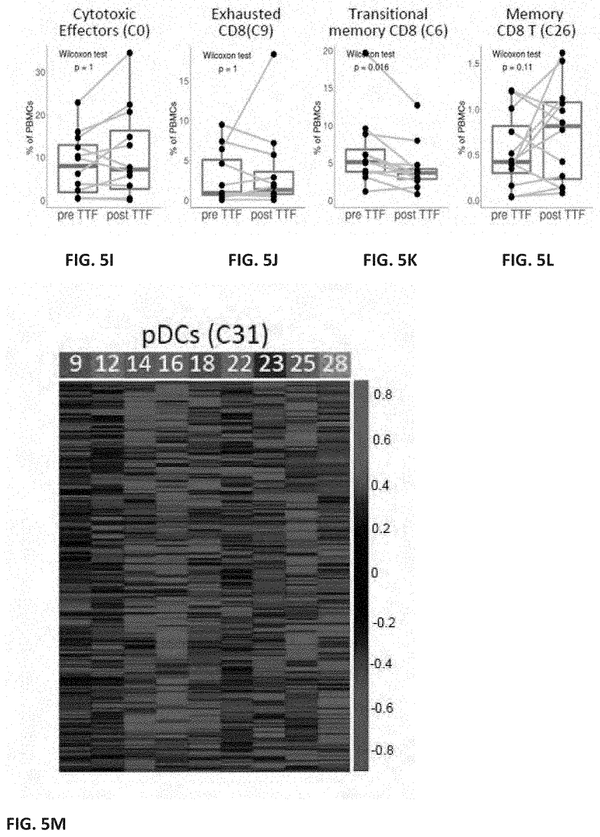

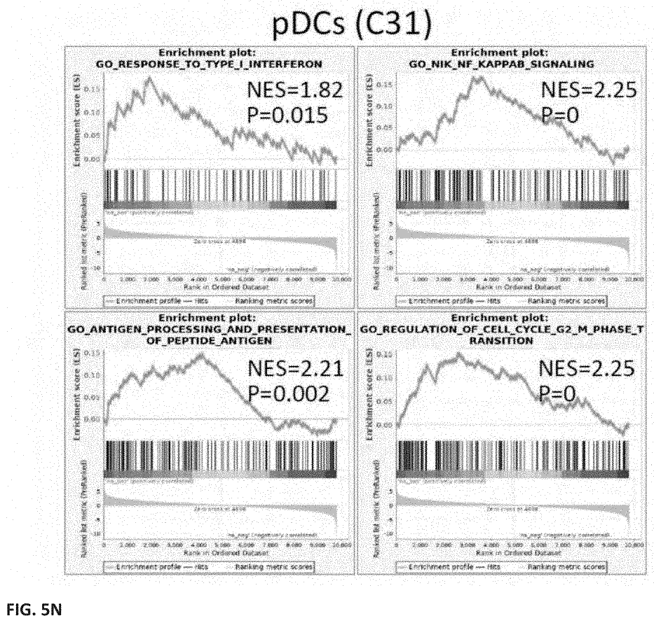

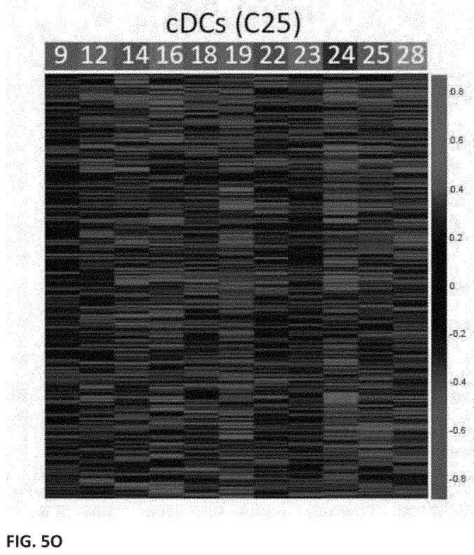

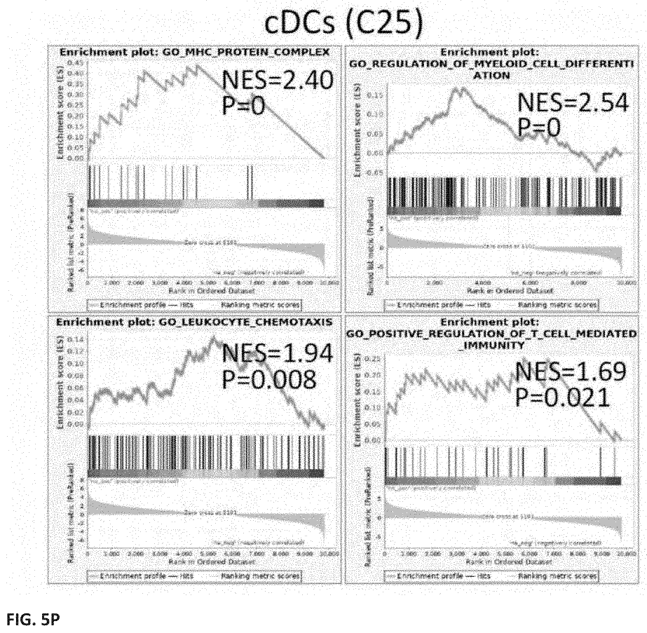

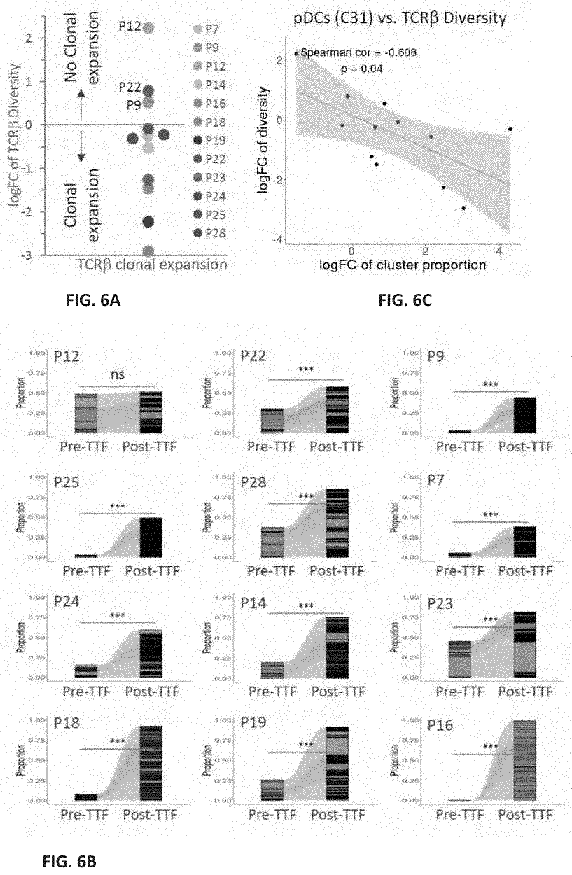

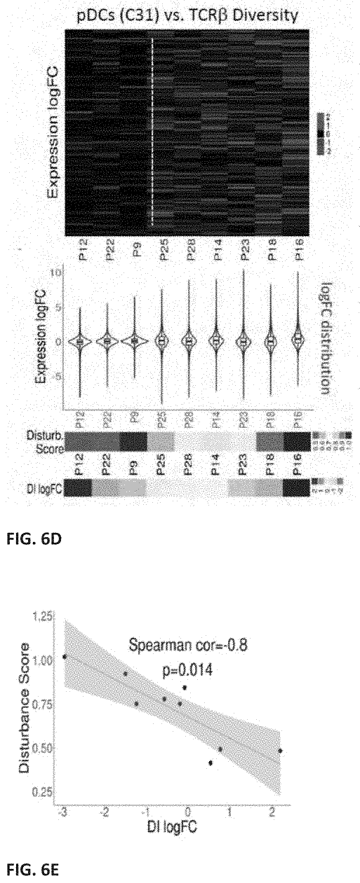

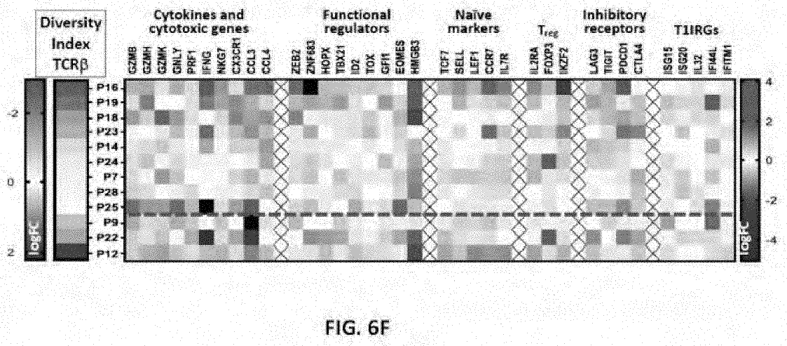

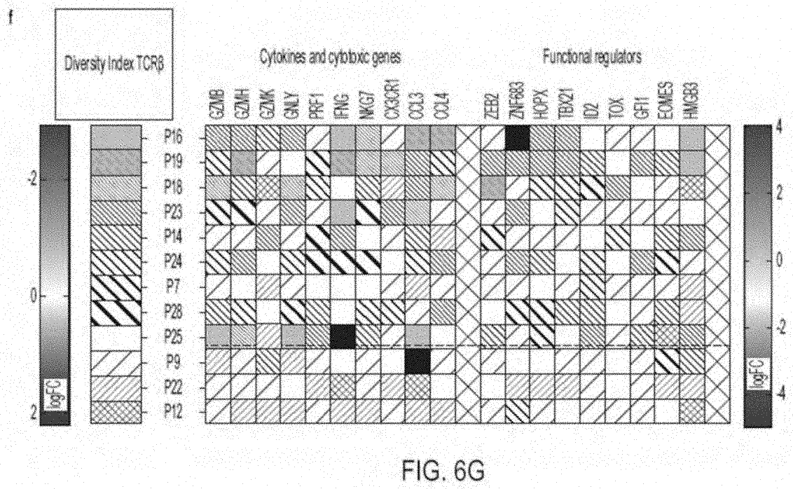

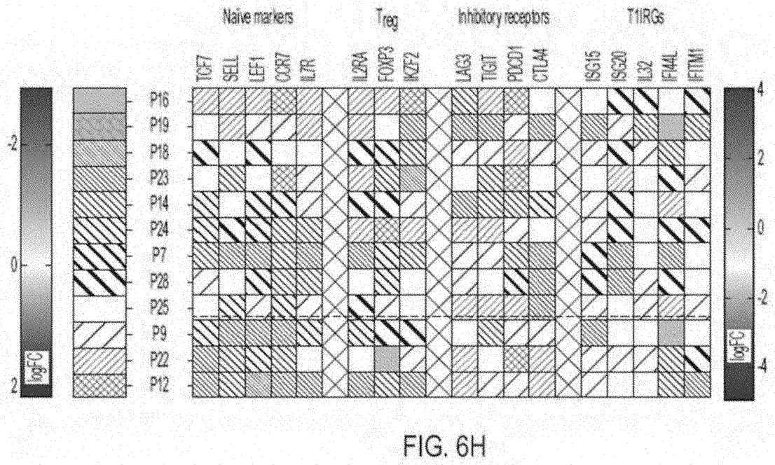

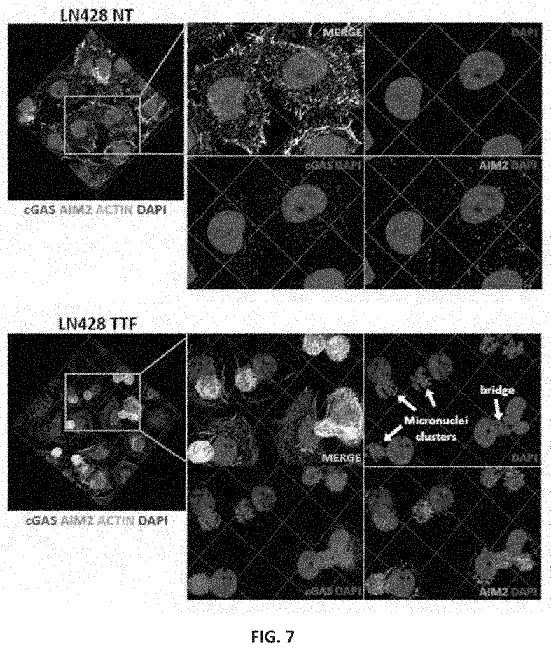

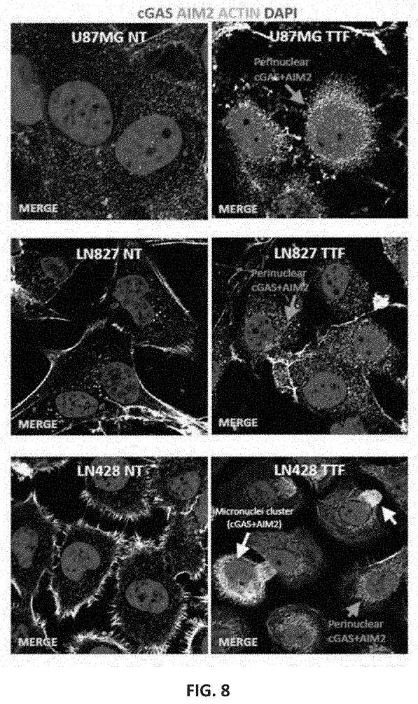

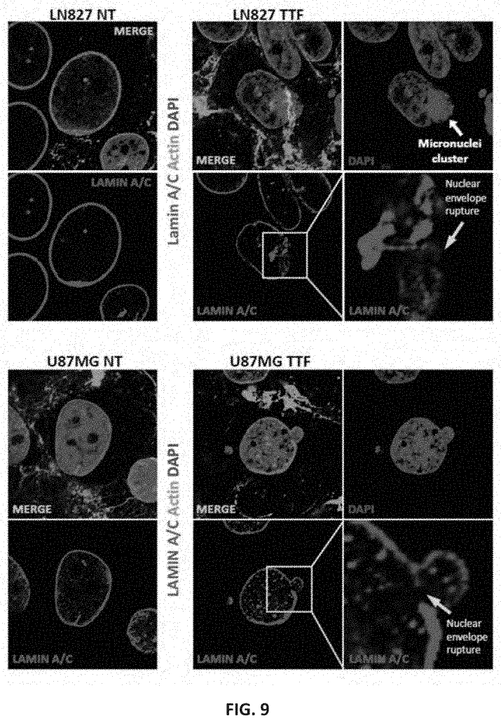

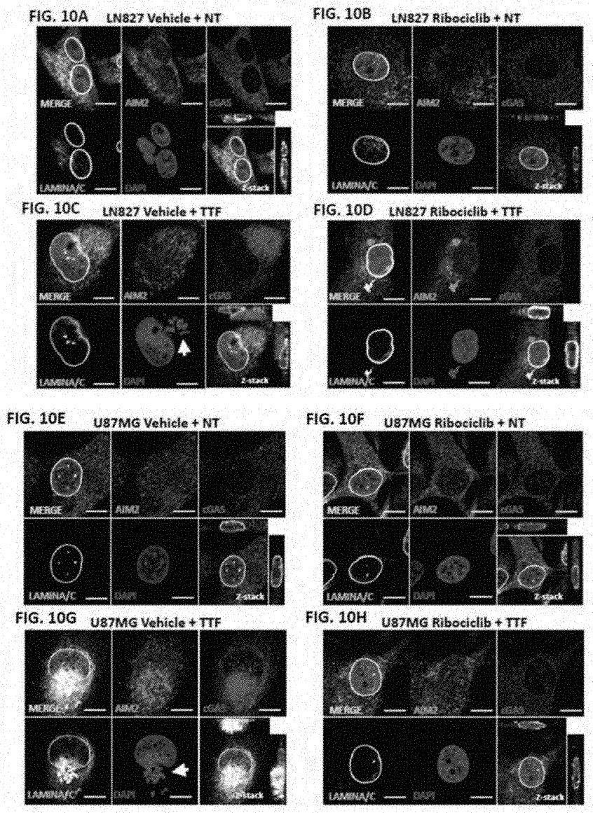

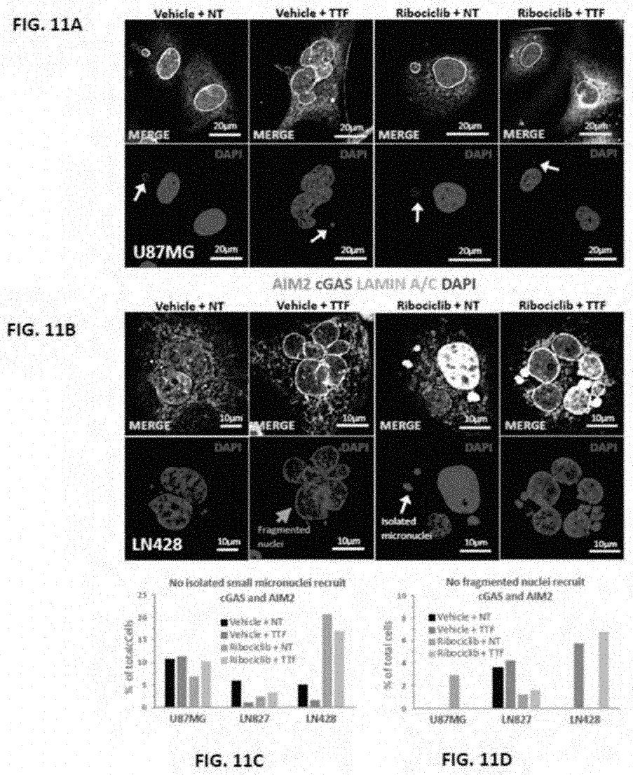

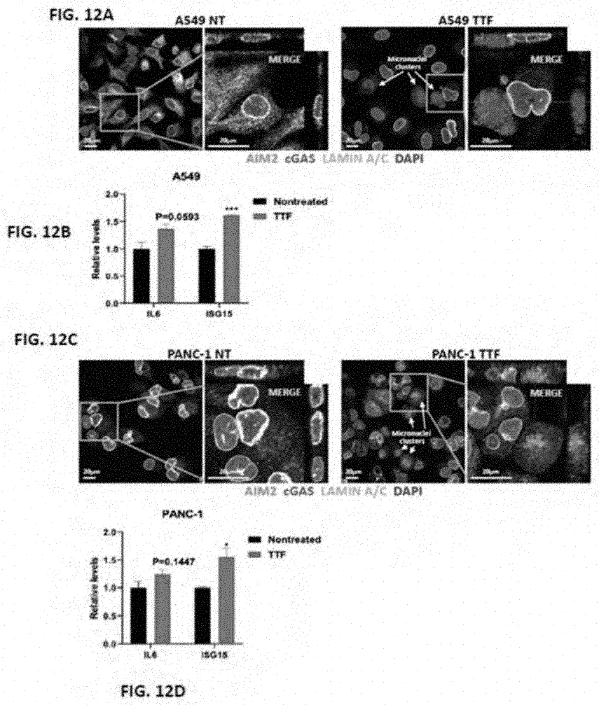

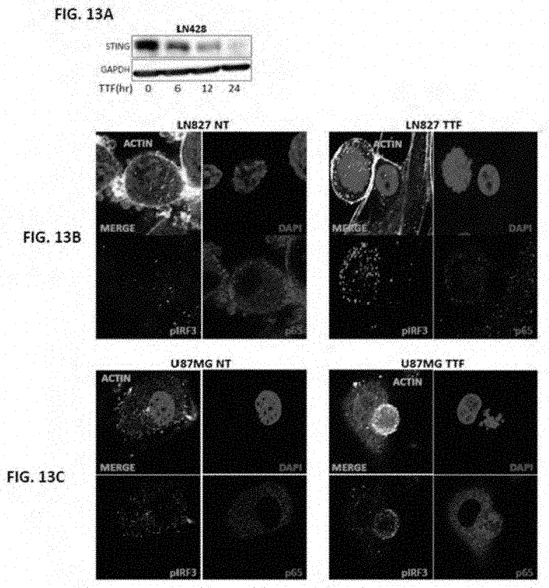

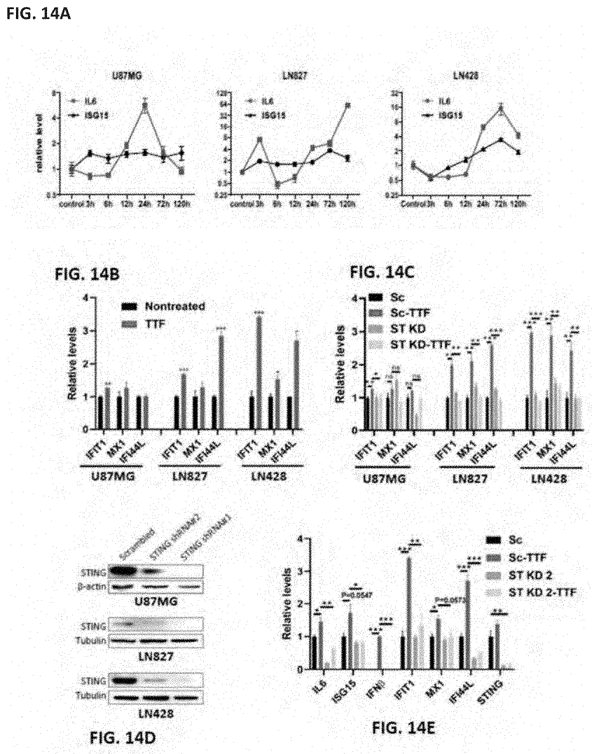

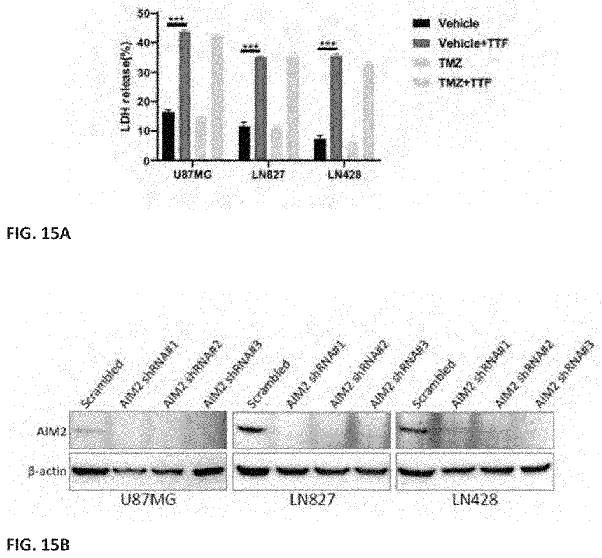

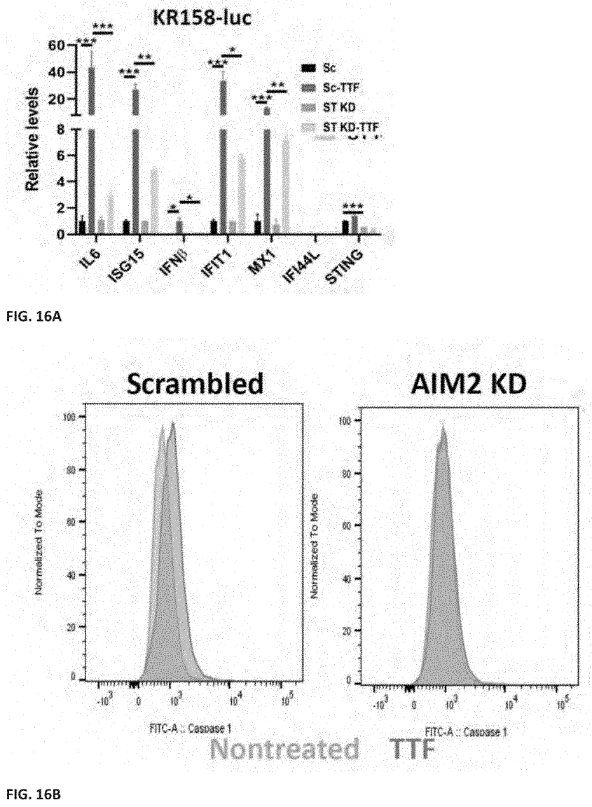

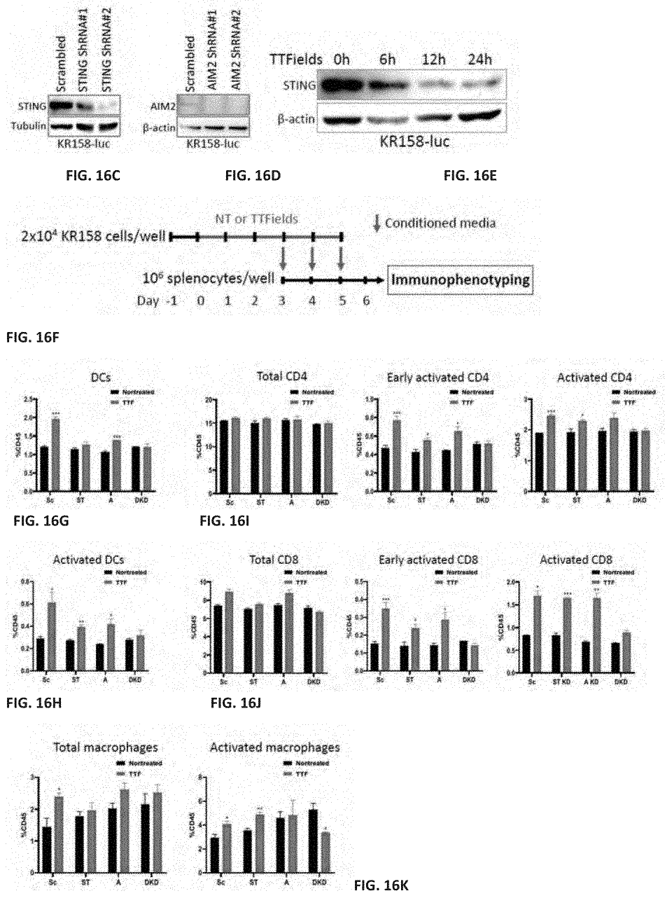

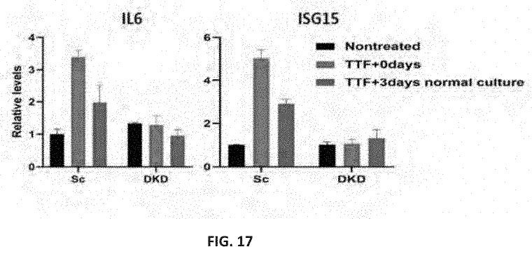

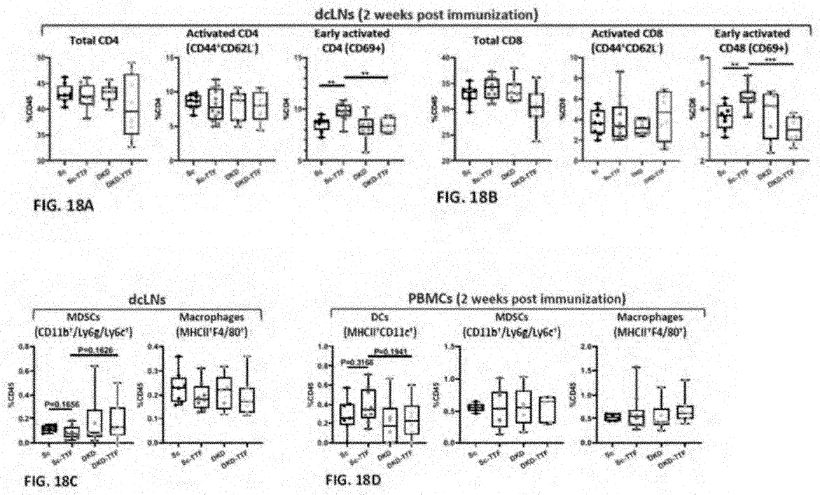

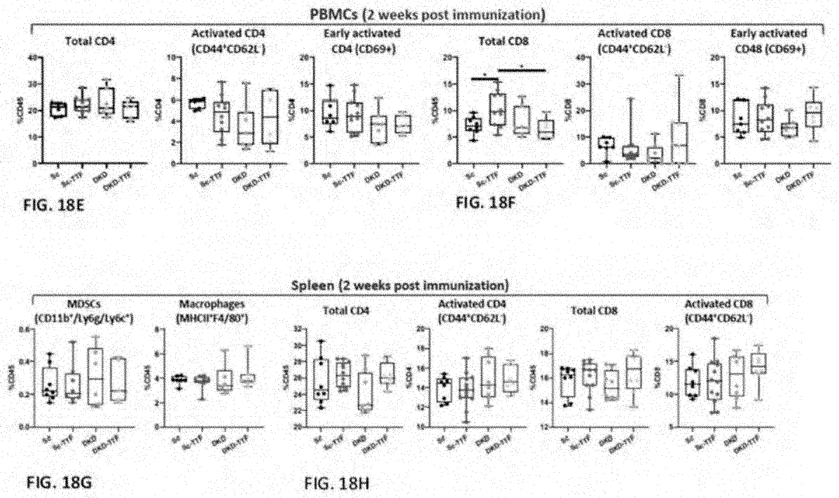

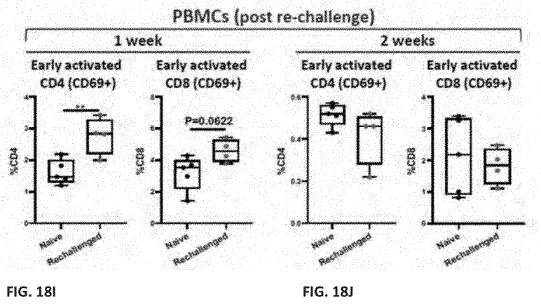

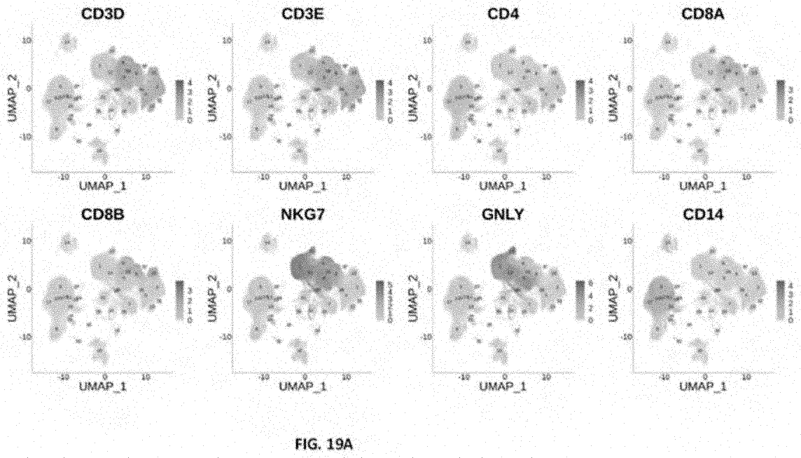

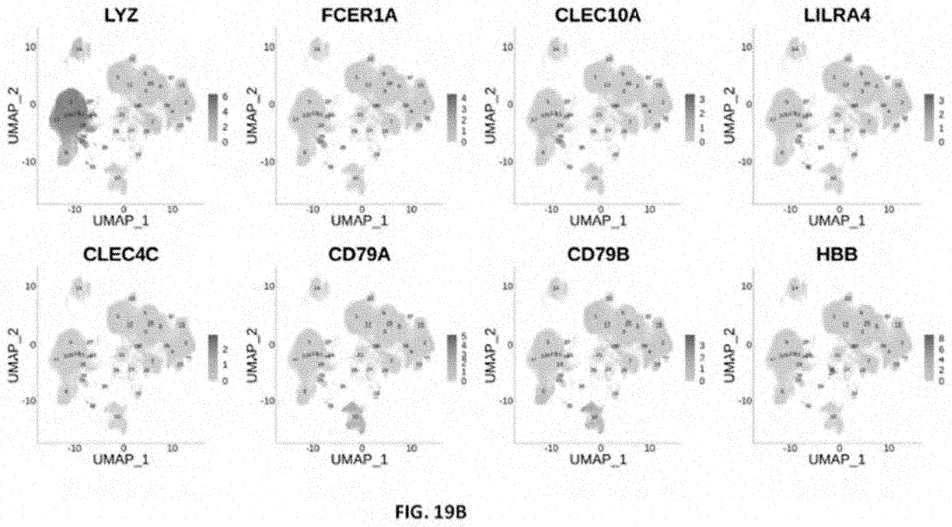

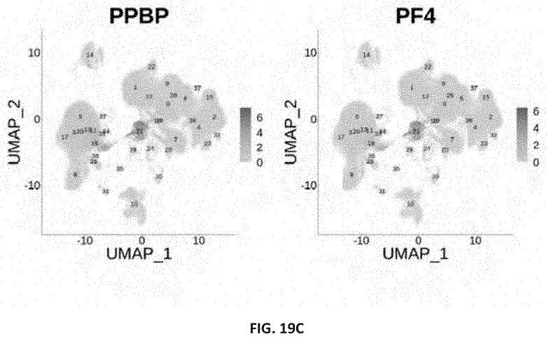

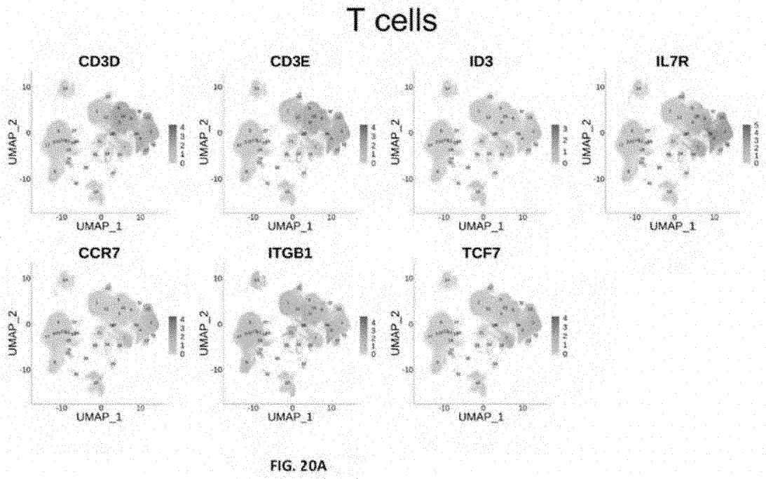

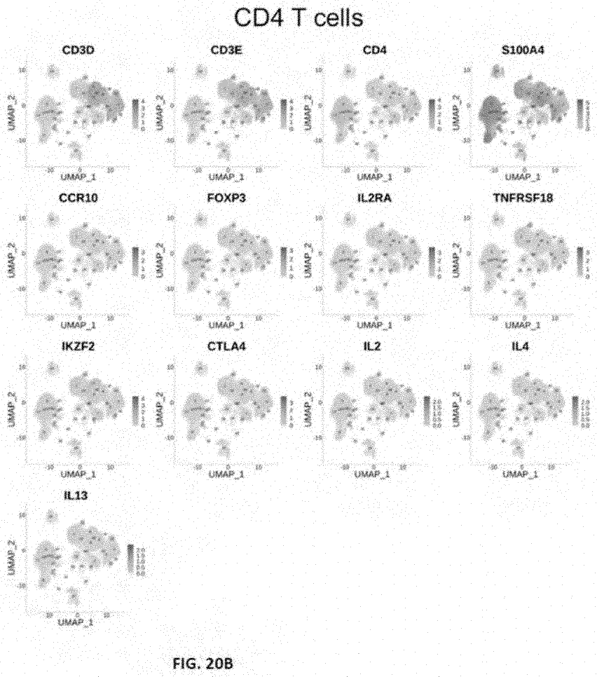

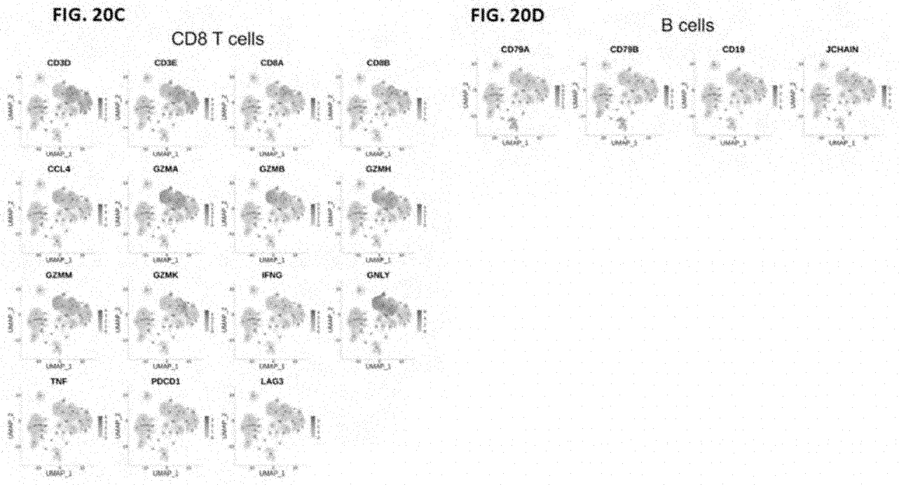

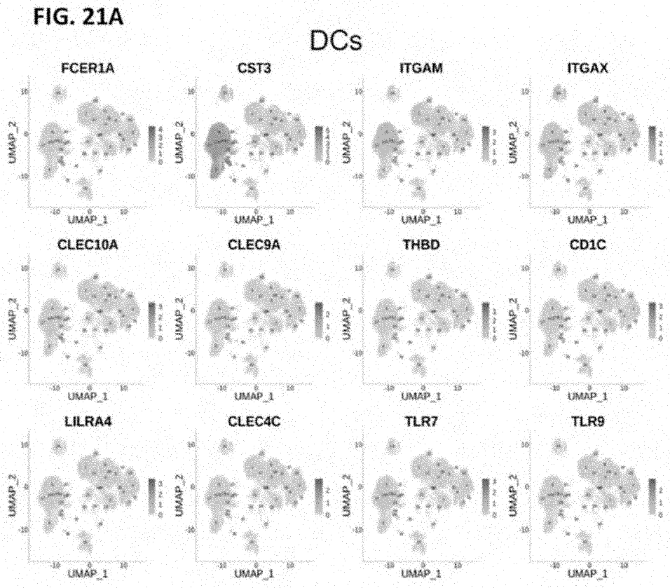

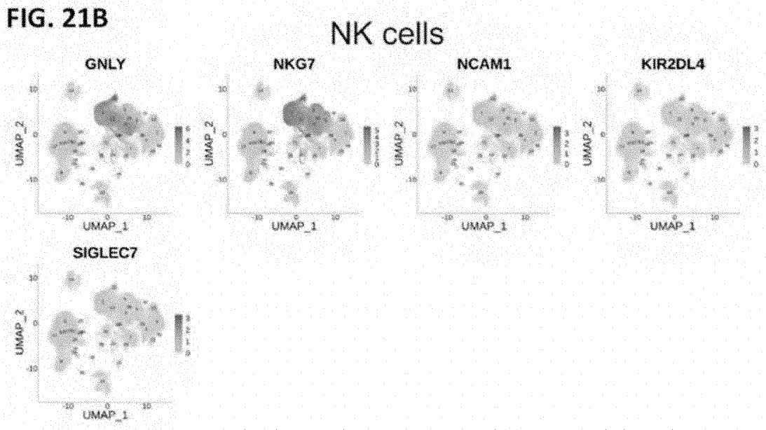

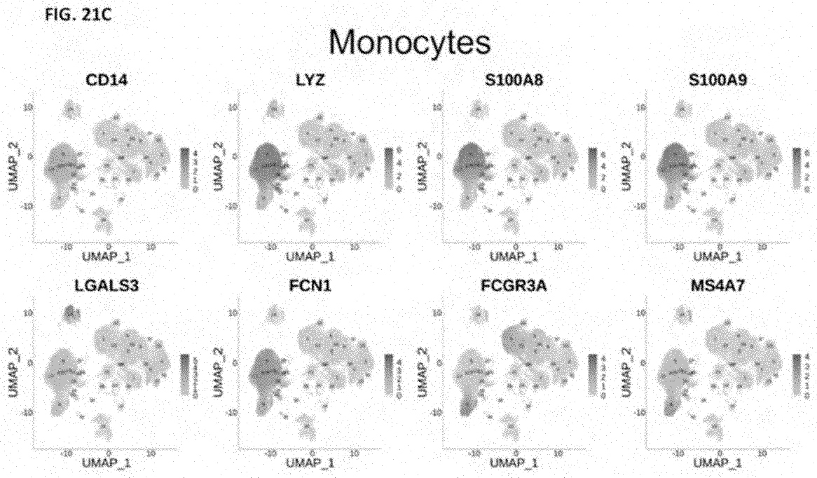

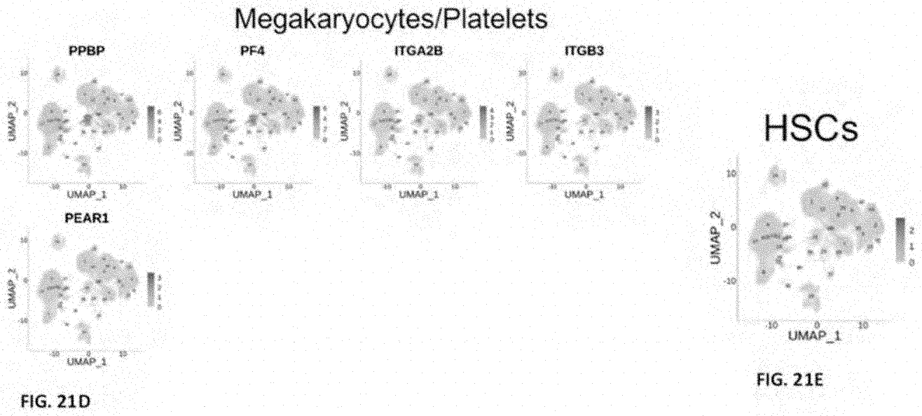

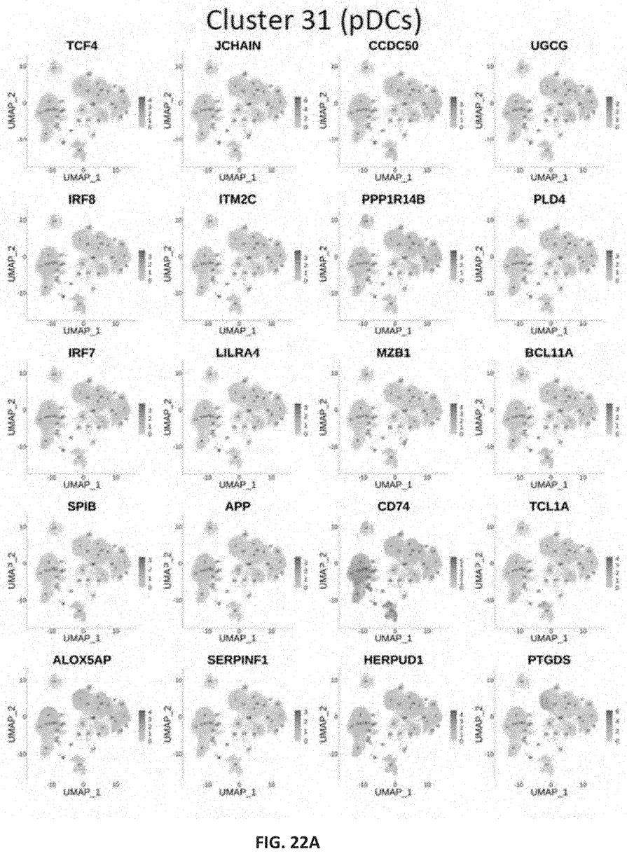

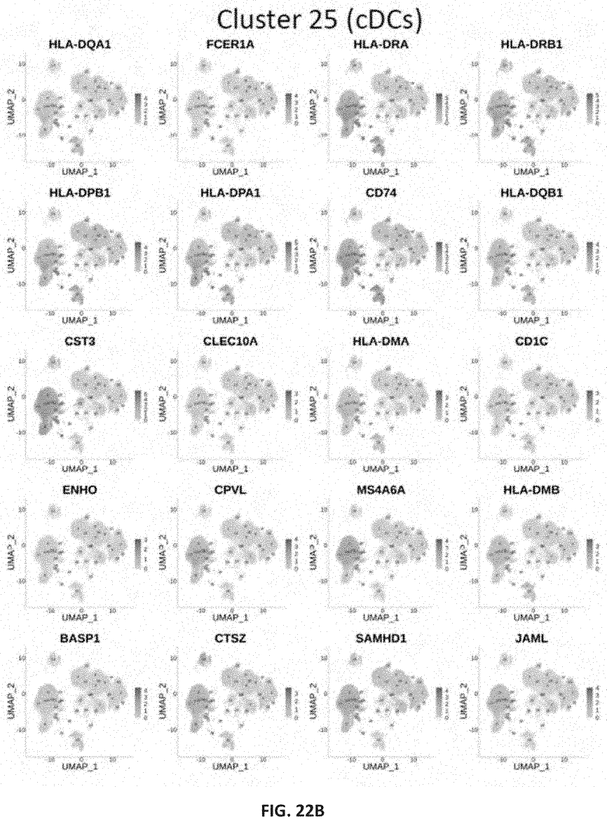

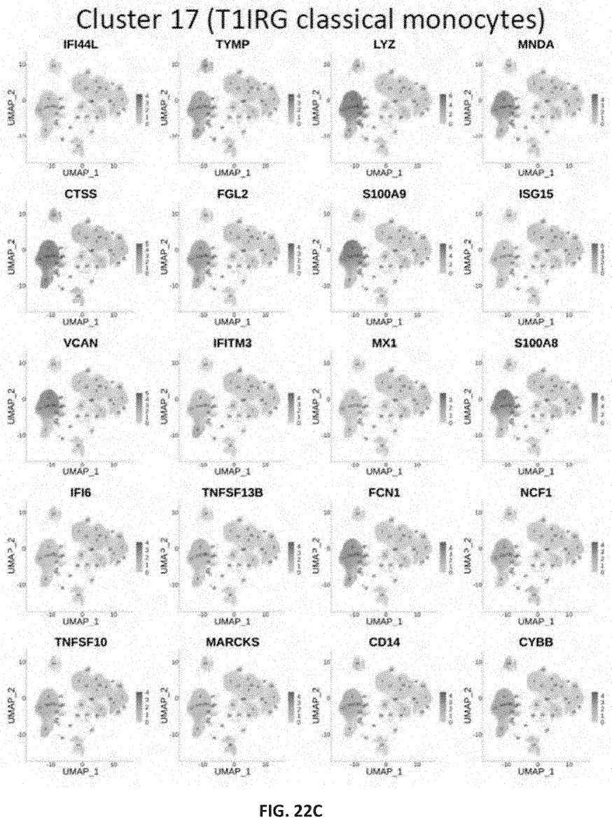

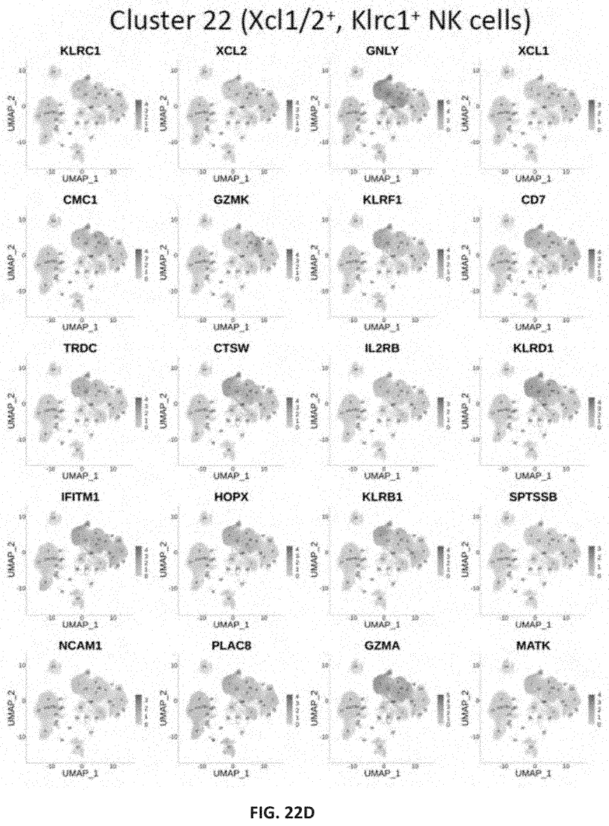

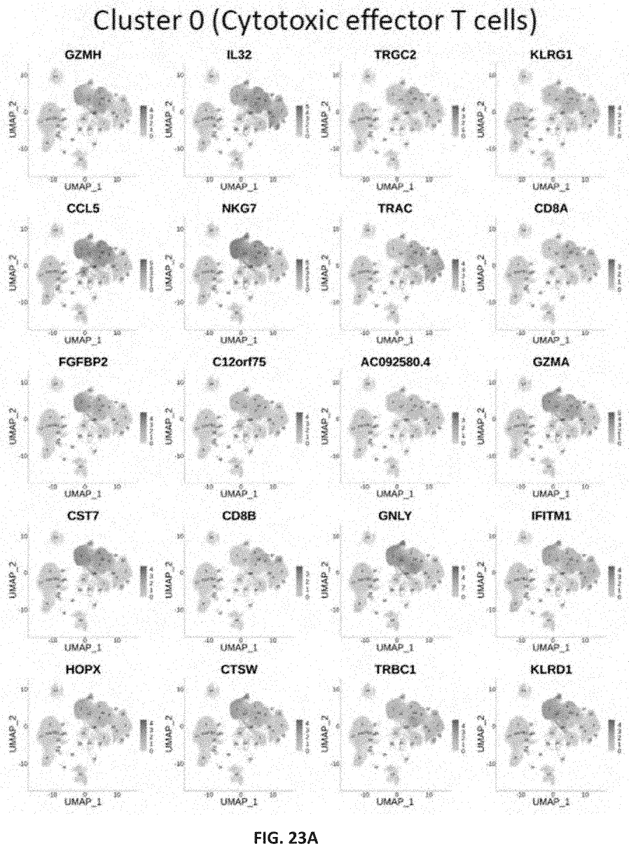

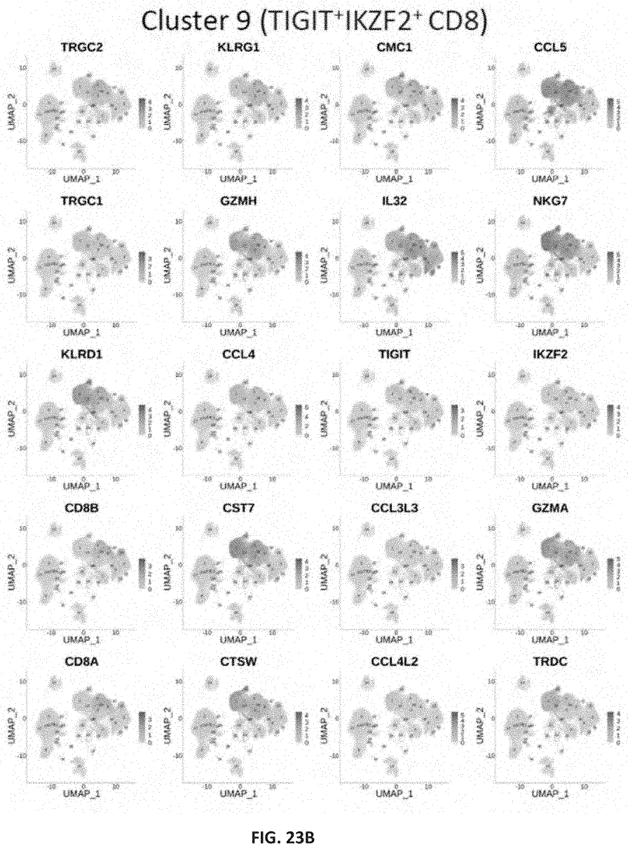

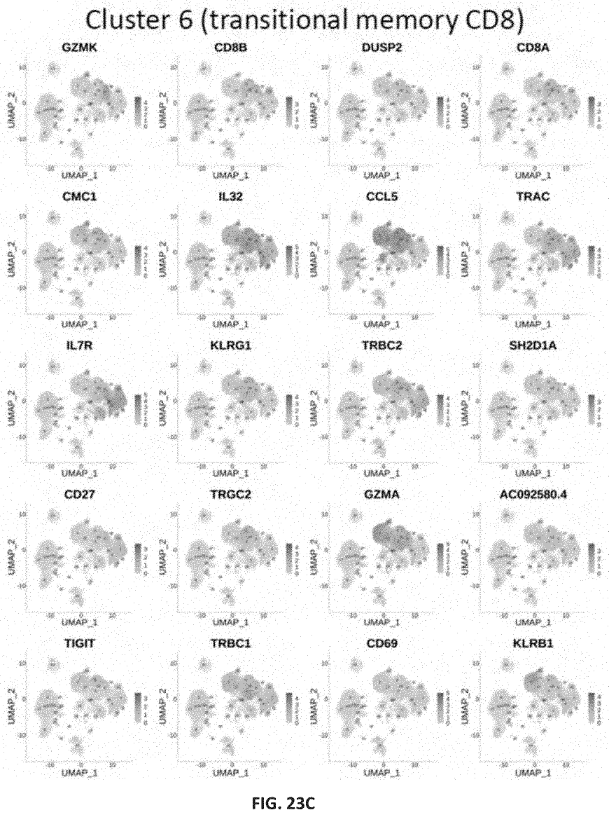

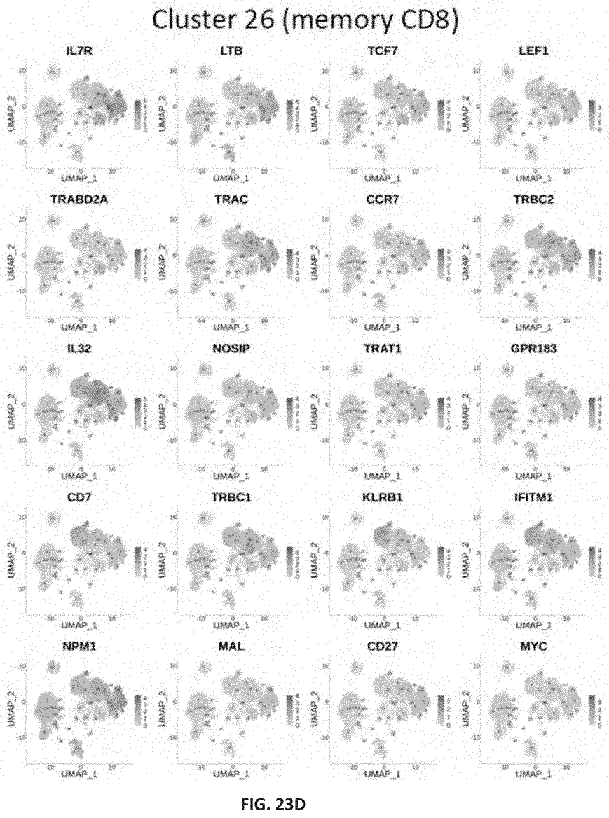

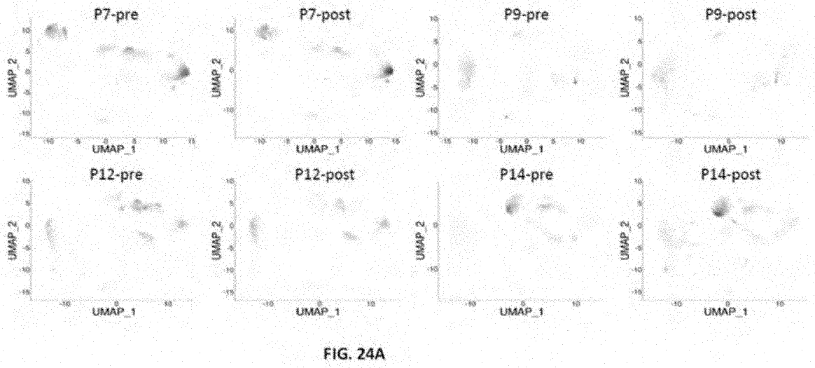

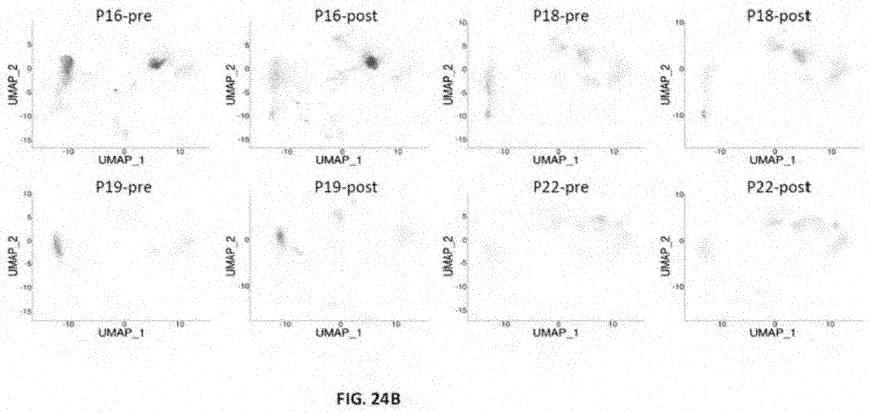

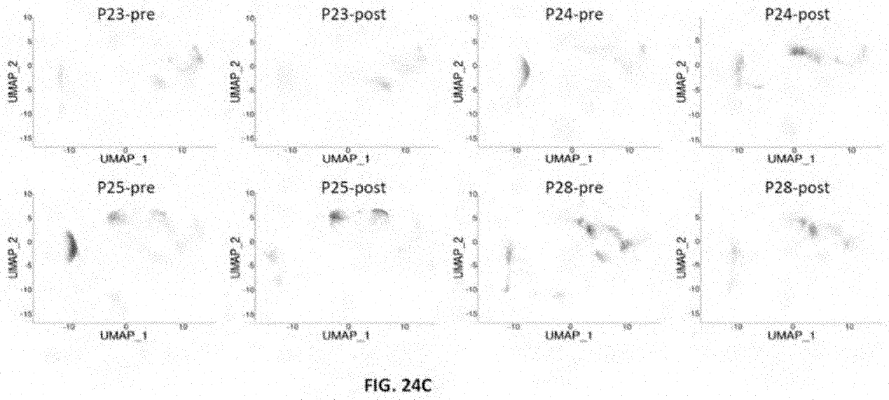

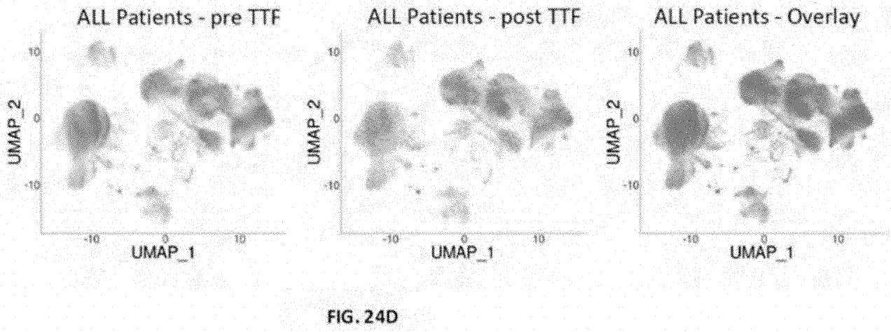

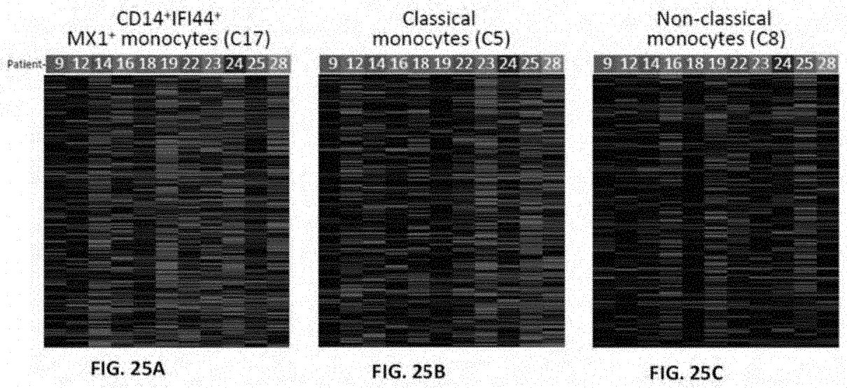

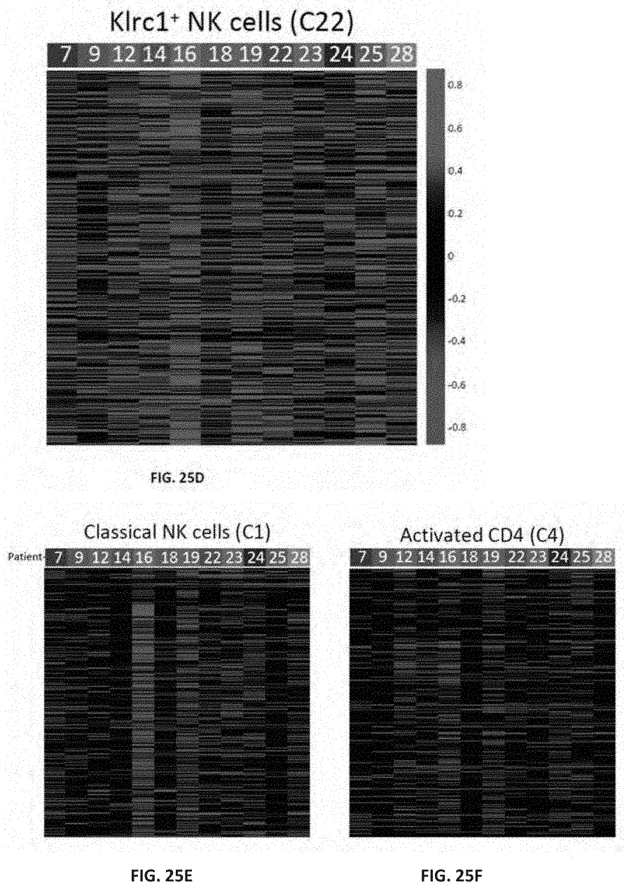

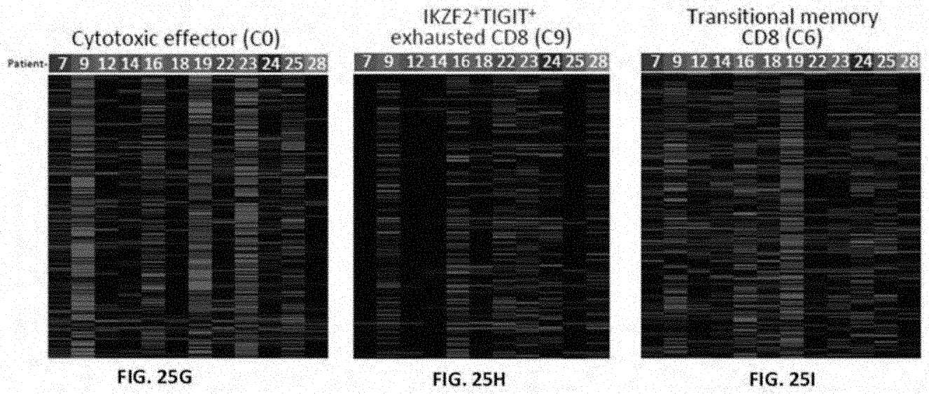

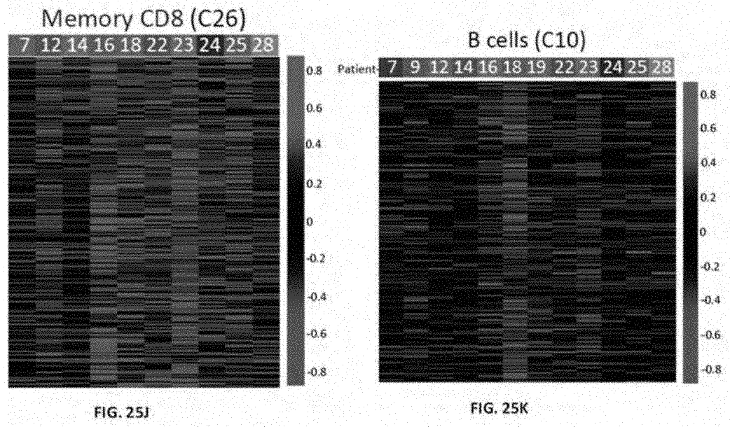

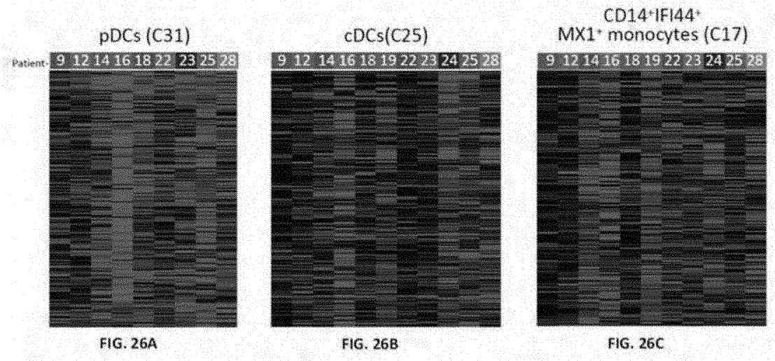

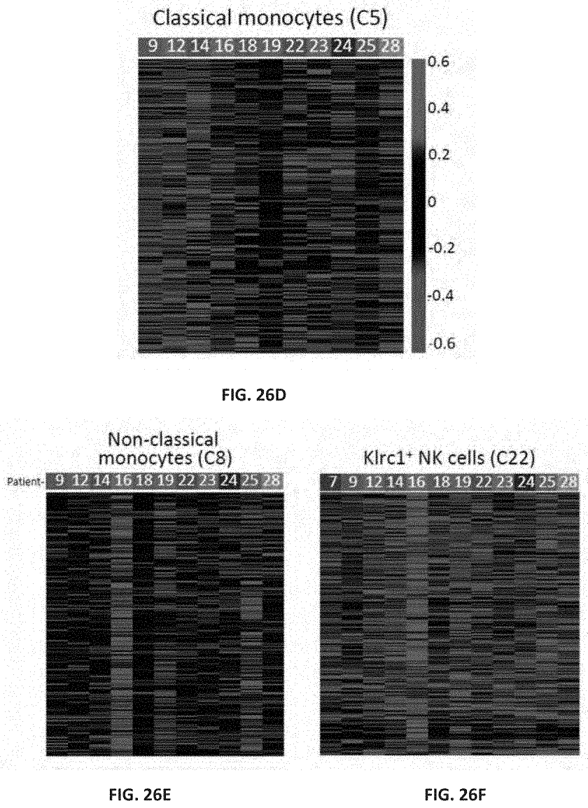

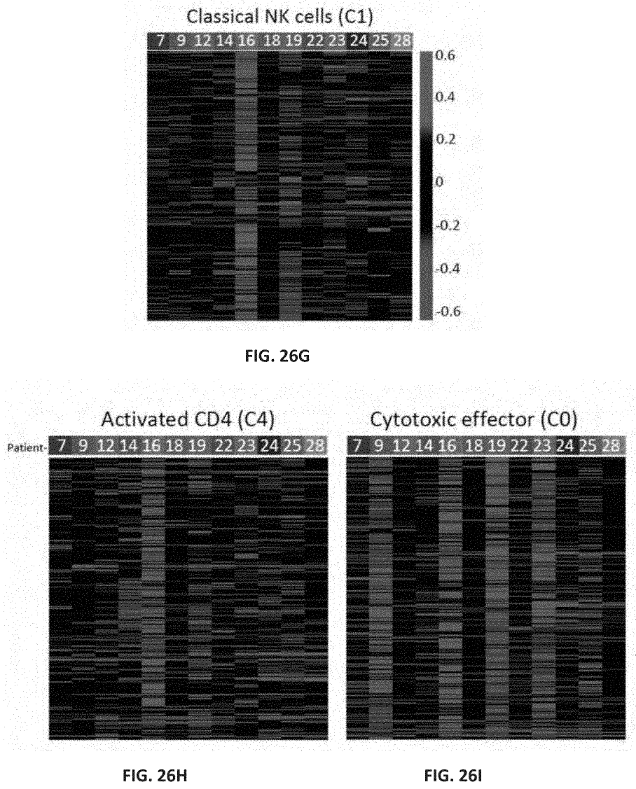

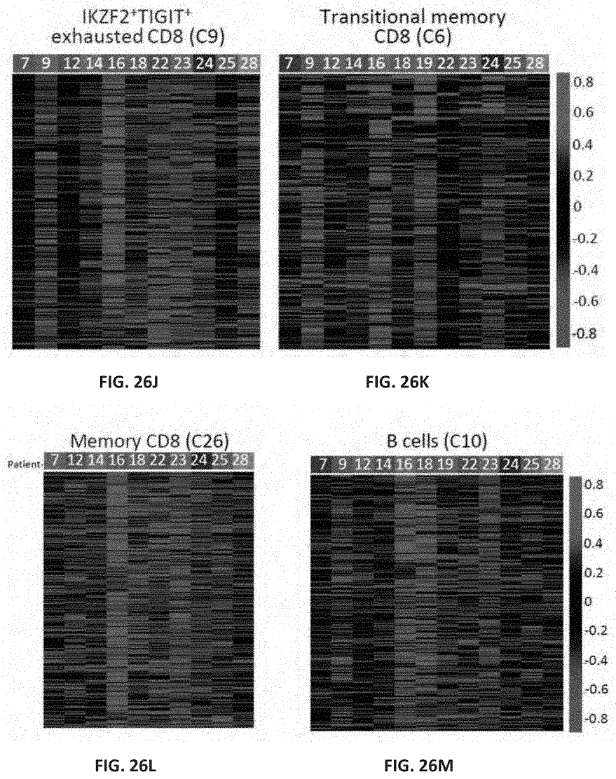

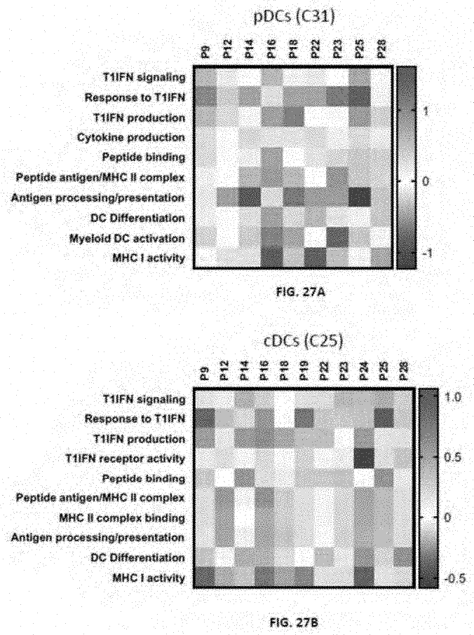

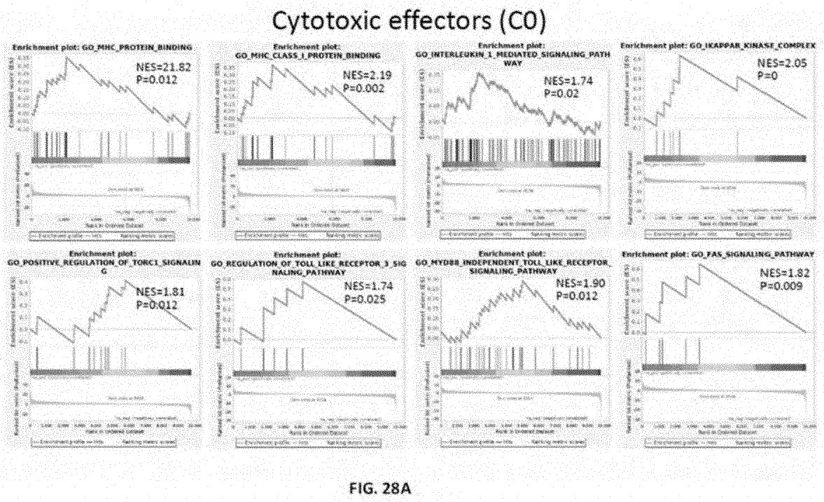

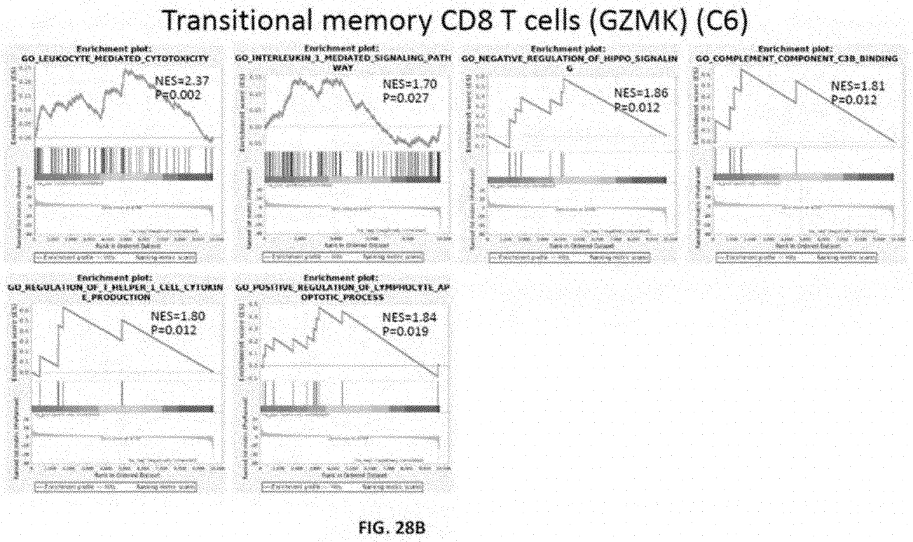

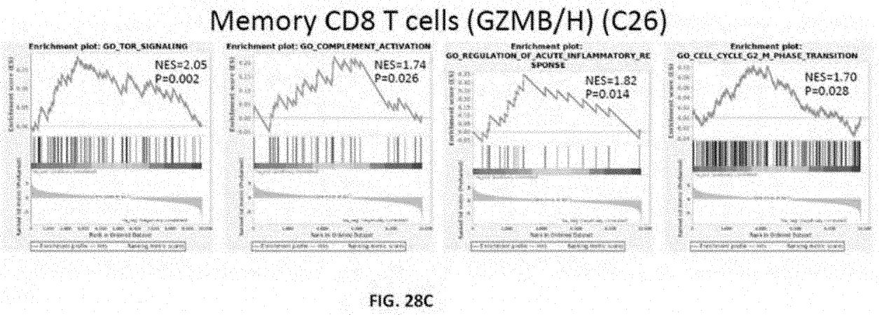

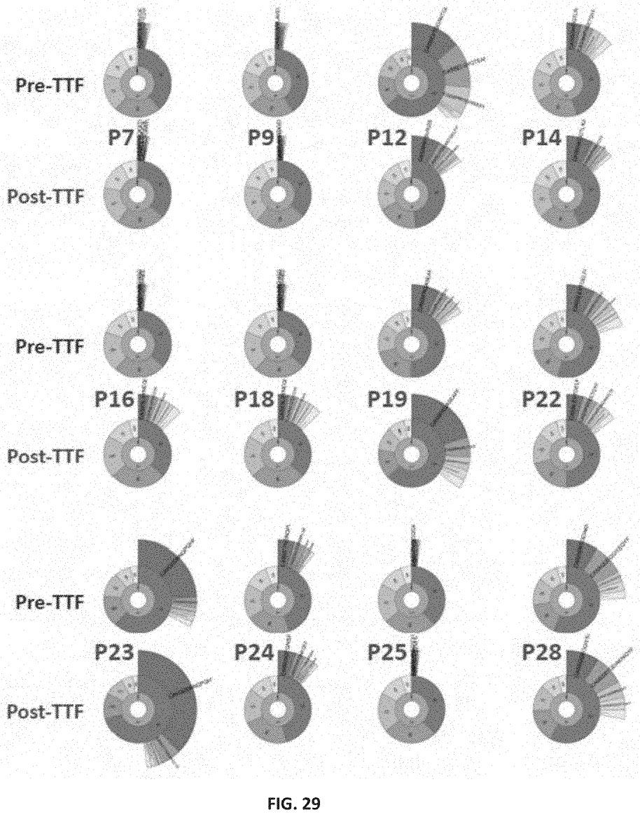

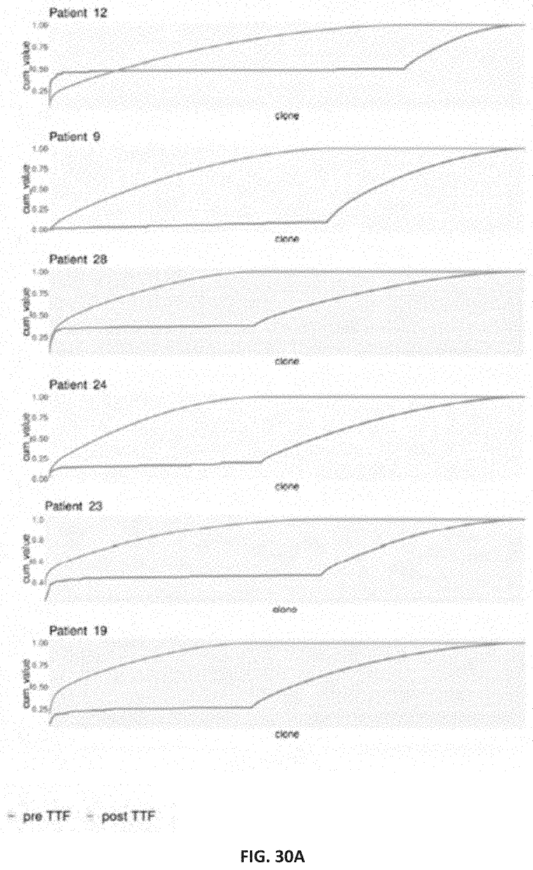

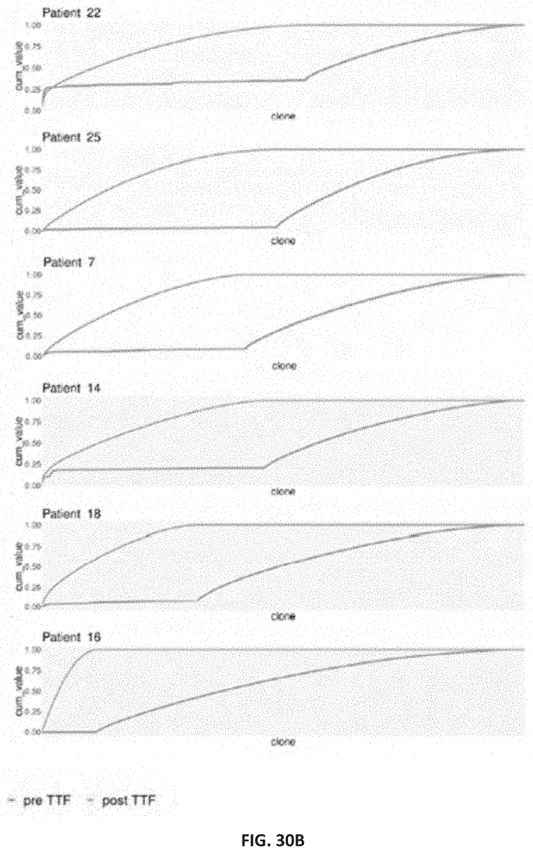

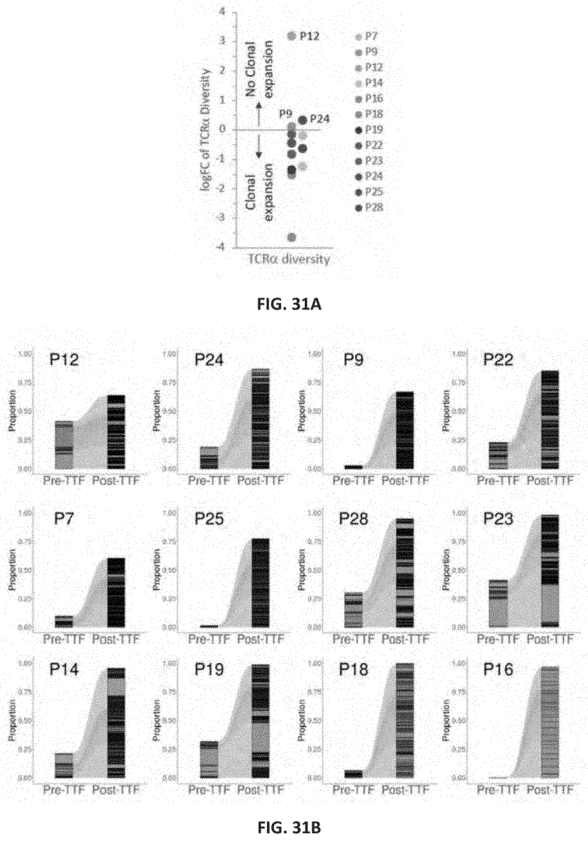

FIG. 4H provides representative photographs showing immunofluorescent staining for CD8 and CD3 and counterstaining for DAPI of orthotopic brain tumors harvested from the same animals used for the experiments described in FIG. 18F and FIG. 18G;

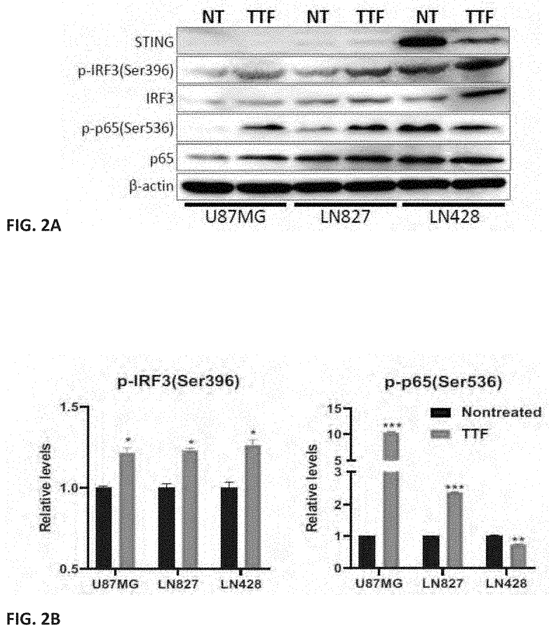

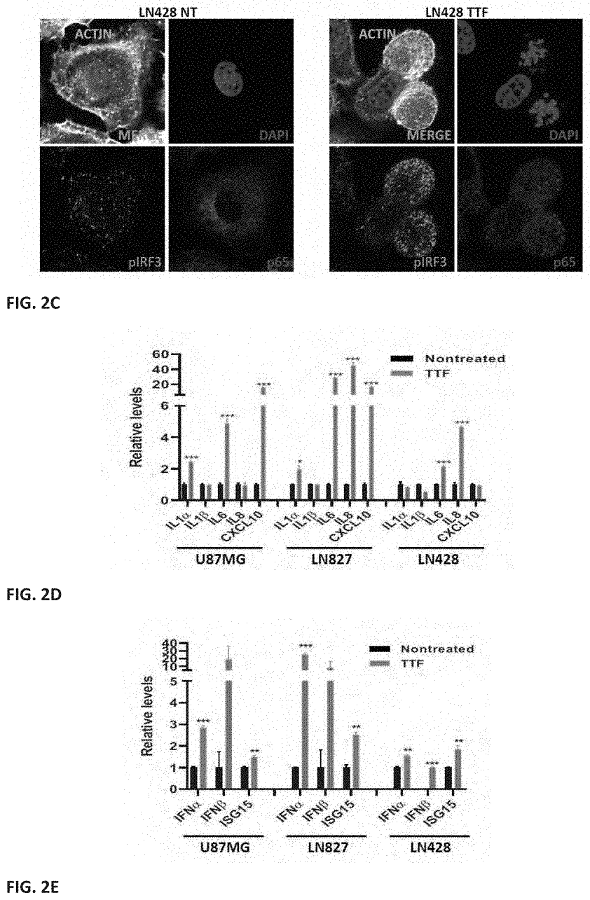

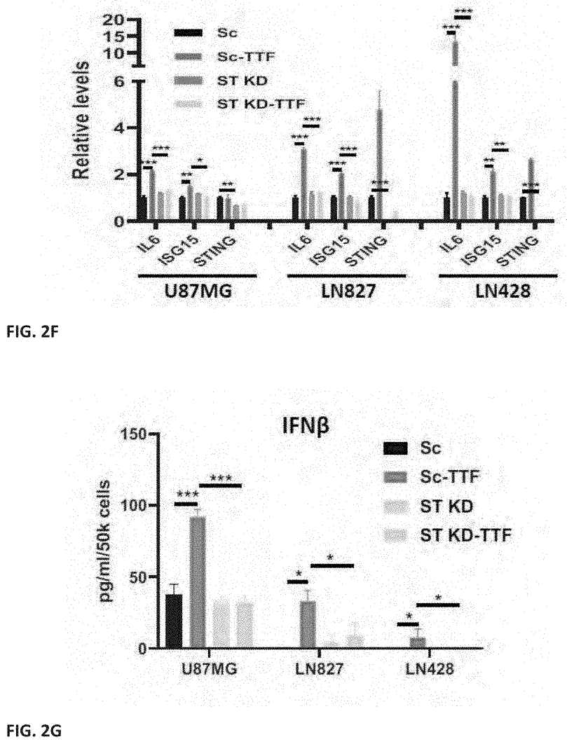

FIG. 4I, FIG. 4J, FIG. 4K, and FIG. 4L provide exemplary combo box and whisker and dot plots showing immunophenotyping for total DCs and fully activated (CD44⁺, CD62L⁻) CD4⁺ and CD8⁺ T cells in PBMCs of surviving Sc-TTF-immunized animals at 1 (FIG. 4I) and 2 (FIG. 4J) weeks post re-challenge with KR158-luc as compared to a new naïve cohort implanted with the same KR158-luc cell;

FIGS. 5A-5P show TTFields treatment correlates with activation of T1FN target immune cells in GBM patients. FIG. 5A provides an exemplary diagram detailing adjuvant TTFields treatment in patients with newly diagnosed GBM;

FIG. 5B provides an exemplary heatmap of expression levels of the indicated gene set implicated in various T cell fates and functions providing the basis for annotations of the indicated major T cell clusters;

FIG. 5C provides an exemplary colored cell cluster map at Resolution 1 using the graph-based cell clustering technique UMAP to resolve 38 major immune cell types and subtypes in the scRNA-seq dataset of PBMCs in 12 GBM patients;

FIG. 5D provides an exemplary overlay of pre-TTFields (pre-TTF-green) and post-TTF (orange) UMAP plots showing post-TTF changes in both proportions and expression (purple broken line) and expression only without proportional change (blue broken line) of the indicated key clusters;

FIG. 5E provides an exemplary heatmap of mean expression levels of the T1IRG pathway GO:0034340 at the single cell level in pre-TTF and post-TTF PBMCs;

FIG. 5F, FIG. 5G, FIG. 5H, FIG. 5I, FIG. 5J, FIG. 5K, and FIG. 5L provide exemplary combo box and whisker and paired dot plots showing the proportions of the indicated clusters as a percentage of total PBMCs in pre-TTF and post-TTF PBMCs;

FIG. 5M and FIG. 5O provide exemplary heatmaps of gene expression showing log FC of post-TTF expression of all-genes compared to pre-TTF expression of all genes in pDCs (FIG. 5M) and cDCs (FIG. 5O) in patients with detectable pre- and post TTF counts in the respective cell types;

FIG. 5N and FIG. 5P provide exemplary gene set enrichment analysis (GSEA) of the indicated GO pathways in pDCs (FIG. 5N) and cDCs (FIG. 5P) comparing between pre and post TTFields treatment of the sample patients in FIG. 5M and FIG. 5O (NES: normalized enrichment score);

FIGS. 6A-6H show TTFields treatment correlates with activation of adaptive immunity in GBM patients. FIG. 6A provides an exemplary dot plot of log FC of the Simpson Diversity Index (DI) of TCRb showing TCRb clonal expansion after TTFields treatment (negative DI log FC) in 9 of 12 patients;

FIG. 6B provides exemplary 2D area charts of the 200 most abundant TCRb clones in post TTFields T cells as compared to their proportions in pre-TTFields T cells showing clonal expansion in 11 of 12 patients;

FIG. 6C provides an exemplary scatter plot of log FC of DI vs. log FC of the proportion of Cluster 31 (pDCs) in 12 patients showing a moderate negative correlation (Spearman correlation coefficient r=−0.608; p=0.04);

FIG. 6D (top panel) provides an exemplary heatmap of gene expression log FC between pre-TTF and post-TTF treatment across 9 patients who had detectable pre-TTFields pDC counts, the middle panel provide a violin plot of gene expression log FC distribution across the 9 patients, and the bottom panels provide a heatmap of Disturbance Score, defined as the mean of absolute gene expression log FC vs. a heatmap of TCRb DI log FC across the 9 patients ordered in decreasing DI log FC;

FIG. 6E provides an exemplary scatter plot of TCRb DI log FC vs. Disturbance Score showing a strong negative correlation (Spearman correlation coefficient r=−0.8, p=0.014);

FIG. 6F provides an exemplary heatmap of gene expression of the same gene set used for T cell cluster annotations in the 12 patients ordered in increasing TCRb DI log FC showing a gene signature of adaptive immune induction by TTFields in GBM patients;

FIG. 6G and FIG. 6H correspond to FIG. 6F with details to convey more clearly features present in the original color version thereof;

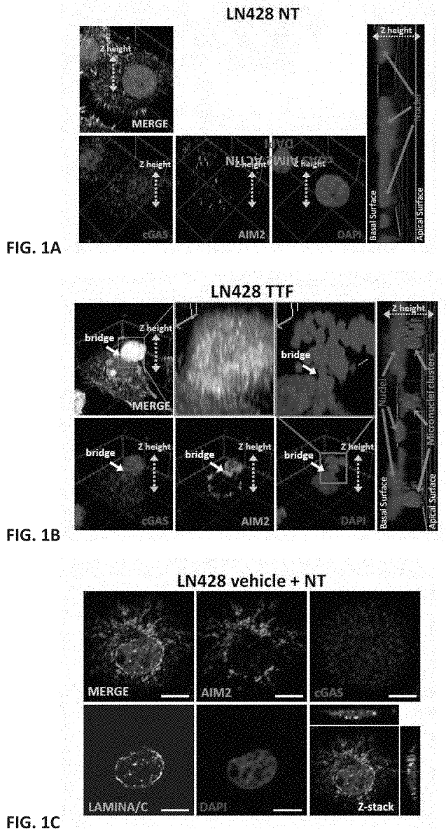

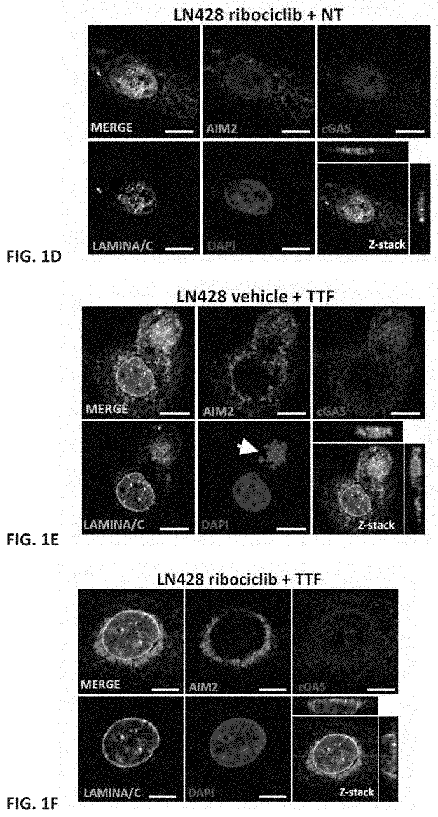

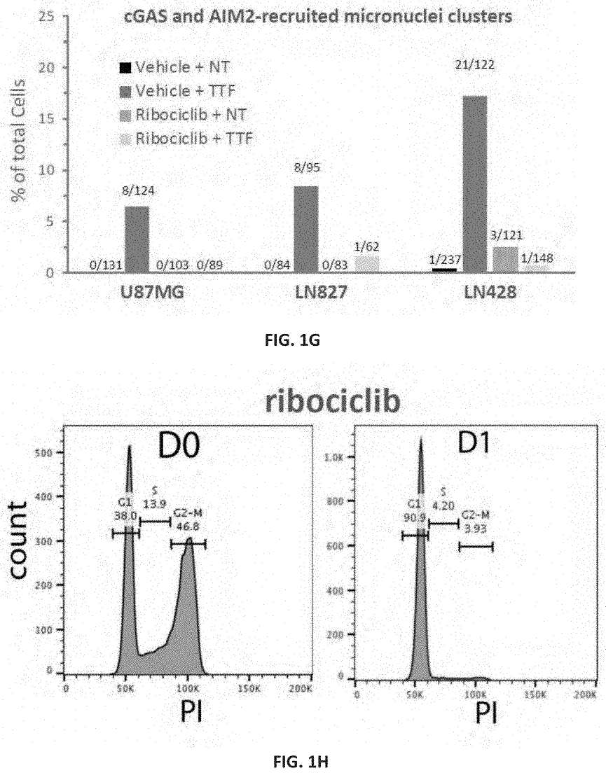

FIG. 7, supporting FIGS. 1A-1B, show TTFields-induced micronuclei clusters recruit cGAS and AIM2. FIG. 7 provides exemplary confocal images with wider fields of view showing immunofluorescent staining of cGAS and AIM2 with β-Actin for cytoplasmic outline and DAPI for nuclear counter-staining in LN428 GBM cells either non-treated (NT) or treated with TTFields (TTF) for 24 hours;

FIG. 8, supporting FIGS. 1A-1B, show TTFields induce focal rupture in the nuclear envelope, through which nuclear content protrudes to form cytosolic naked micronuclei clusters. FIG. 8 provides exemplary confocal images showing immunofluorescent staining of cGAS and AIM2 with β-actin for cytoplasmic outline and DAPI for nuclear counter-staining in the 3 indicated GBM cell lines either non-treated (NT) or treated with TTFields (TTF) for 24 hours;

FIG. 9, supporting FIGS. 1A-1B, shows TTFields induce focal rupture in the nuclear envelope, through which nuclear content protrudes to form cytosolic naked micronuclei clusters. FIG. 9 provides exemplary confocal images showing immunofluorescent staining of LAMIN A/C with β-actin for cytoplasmic outline and DAPI for nuclear counter-staining in U87MG and LN827 GBM cells either non-treated (NT) or treated with TTFields (TTF) for 24 hours;

FIGS. 10A-10H, supporting FIGS. 1A-1H, show TTFields-induced micronuclei clusters recruit cGAS and AIM2 and require S phase entry. FIGS. 10A-10H provide exemplary confocal images with z stack showing immunofluorescent staining of cGAS, AIM2, and LAMIN A/C with DAPI counter-staining in LN827 (FIGS. 10A-10D), U87MG (FIGS. 10E-10H) GBM cells that were pretreated with either the vehicle (FIG. 10A, FIG. 10C, FIG. 10E, FIG. 10G) or ribociclib (FIG. 10B, FIG. 10D, FIG. 10F, FIG. 10H) to induce G1 arrest, then non-treated (FIGS. 10A-10B, FIGS. 10E-10F) or treated with TTFields for 24 hours (FIGS. 10C-10D, FIGS. 10G-10H), demonstrating that S phase entry is required for TTFields-induced cytosolic micronuclei clusters;

FIGS. 11A-11D, supporting FIGS. 1A-1H, show isolated cytosolic micronuclei and fragmented nuclei are formed independent of TTFields and shielded by a lamin A/C-based nuclear membrance, and do not recruit cGAS and AIM2. FIGS. 11A-11D provide exemplary confocal images showing immunofluorescent staining for cGAS, AIM2, and LAMIN A/C with DAPI counter-staining in U87MG (FIG. 11A), LN428 (FIG. S5b) GBM cells that were pretreated with either the vehicle or ribociclib to induce G1 arrest, then non-treated or treated with TTFields for 24 hours;

FIG. 11C and FIG. 11D provide exemplary bar graphs showing percentages of cells with isolated small free-standing cytosolic micronuclei (FIG. 11C) and fragmented nuclei (FIG. 11D) in the 3 indicated GBM cell lines treated with various conditions (FIG. 11A and FIG. 11B);

FIGS. 12A-12D, supporting FIGS. 1A-1H, show TTFields similarly activate the cGAS-STING and AIM2-caspase-1 inflammasomes in other solid tumor cells. FIGS. 12A-12D provide exemplary confocal images with z stack showing that treatment with TTFields at 150 kHz for 24 hours resulted in large cytosolic micronuclei clusters that recruited both cGAS and AIM2 in the lung adenocarcinoma cell line A549 (FIG. 12A) and the pancreatic adenocarcinoma cell line PANC-1 (FIG. 12C), as determined by immunofluorescent staining for cGAS, AIM2, and LAMIN A/C with DAPI counter-staining while both the PIC IL6 and the T1IRG ISG15 were regulated in response to TTFields in these cell lines (FIG. 12B and FIG. 12D);

FIGS. 13A-13C, supporting FIG. 2C, show the cGAS-STING inflammasome is activated with TTFields in GBM cells. FIGS. 13A-13C provide an exemplary radiograph showing immunoblotting for STING in LN428 GBM cells, and showing that STING was rapidly degraded within 6 hours of TTFields exposure;

FIG. 13B and FIG. 13C (supporting FIG. 2C) show that increased concentration and recruitment of p-IRF3 and p65 in large cytosolic micronuclei clusters was detected by immunofluorescent staining and confocal microscopy in LN827 (FIG. 13B) and U87MG FIG. 13C) GBM cells after 24 hour treatment with TTFields;

FIGS. 14A-14E, supporting FIGS. 2D-2G, show PIC's and T1RG's of the cGAS-STING inflammasome are activated by TTFields. FIGS. 14A-14E provide exemplary kinetics of mRNA upregulation of the PIC IL6 and the T1IRG ISG15 in response to TTFields in the 3 indicated GBM cell lines showing a peak in mRNA expression by 72 hours;

FIG. 14B (supporting FIGS. 2D-2G) provides an exemplary bar graph showing relative mRNA upregulation of several additional T1IRGs in the 3 indicated GBM cell lines in response to 24 hour treatment with TTFields;

FIG. 14C (supporting FIGS. 2D-2G) provides an exemplary bar graph showing that TTFields-induced upregulation of additional T1IRGs was also dependent on STING as measured in their mRNA expression levels in the 3 indicated GBM cell lines that express a scrambled (Sc) or STING (ST KD) shRNA, and that were either non-treated or treated with TTFields for 24 hours;

FIG. 14D (supporting FIGS. 2D-2G) provides an exemplary radiograph showing immunoblotting for STING depletion using 2 independent STING shRNAs #1 and #2 in U87MG, LN824, and LN428 GBM cells;

FIG. 14E (supporting FIGS. 2D-2G) shows that independent STING shRNA #2 similarly blunted TTFields-induced upregulation of several representative PICs and T1IRGs in LN428 GBM cells after 24 hour treatment with TTFields;

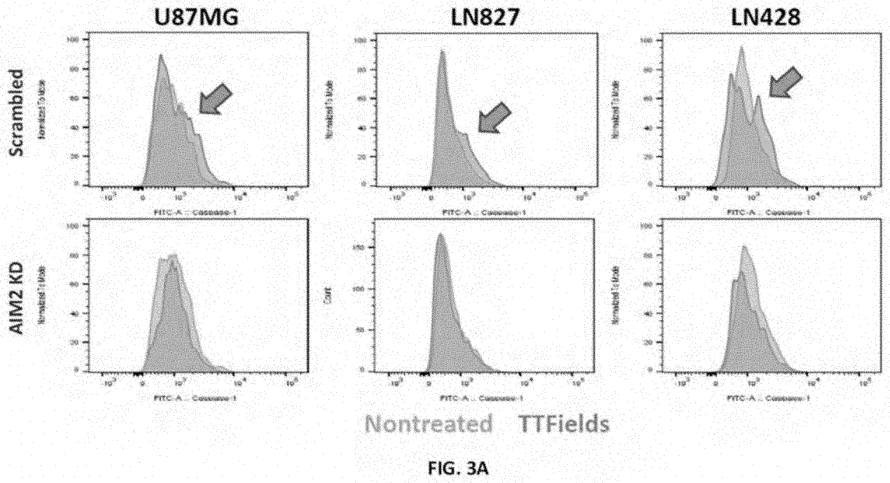

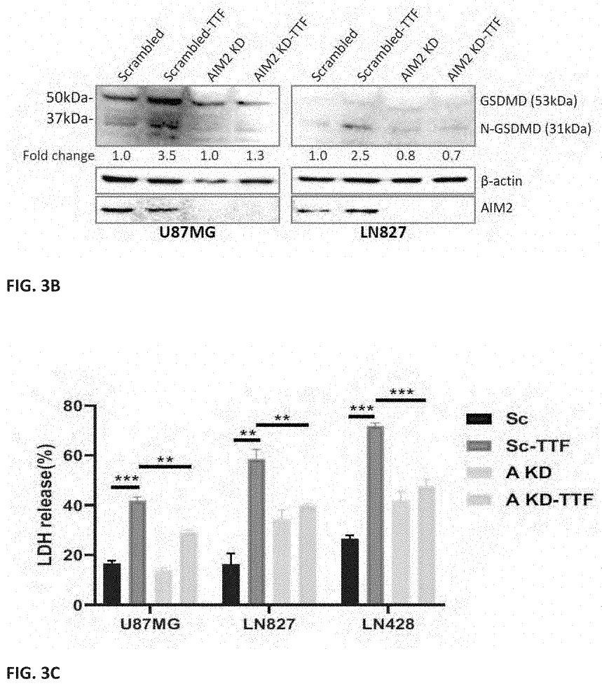

FIGS. 15A-15B, supporting FIG. 3A, show plasma membrane disruption by TTFields and AIM2 shRNA validation. FIGS. 15A-15B provide an exemplary bar graph of an LDH release assay following treatment with TTFields for 24 hours at 200 kHz and TMZ (150 μg/ml) showing that TTFields-induced programmed necrotic cell death is distinct from death caused by TMZ;

FIG. 15B provides exemplary radiographs of immunoblotting for AIM2 showing efficient KD of AIM2 using shRNAs;

FIGS. 16A-16K, supporting FIGS. 4A-4L, show TTFields-induced PICs and T1FNs stimulate DCs and lymphocytes. FIGS. 16A-16K show that TTFields stimulate the cGAS-STING inflammasome in the murine GBM model KR150-luc in a STING and AIM2-dependent manner;

FIG. 16B (supporting FIGS. 4A-4L) shows that TTFields stimulate the AIM2-caspase-1 inflammasome in the murine GBM model KR150-luc in a STING and AIM2-dependent manner;

FIG. 16C and FIG. 16D show that at least 2 shRNAs each for STING (FIG. 16C) and AIM2 (FIG. 16D) were used with similar results;

FIG. 16E provides exemplary radiographs showing immunoblotting for STING in KR158-luc GBM cells rapidly being degraded within 6 hours of TTFields exposure;

FIG. 16F provides an exemplary diagram detailing the co-culture schema where KR158 cells were treated with TTFields for 144 hours and conditioned supernatants collected starting at 72 hours and then daily for the next 3 days to culture splenocytes freshly isolated from syngeneic mice for 3 days, followed by immunophenotyping;

FIGS. 16G-16K provide exemplary bar graphs showing immunophenotyping of all CD45⁺ cells in syngeneic splenocytes co-cultured with conditioned supernatants obtained from KR158 cells with or without Scrambled control (Sc), single STING KD (ST), single AIM2 KD (A) or double STING/AIM2 KD (DKD) shRNA that were either non-treated or treated with TTFields for 24 hours for total DCs (MHCII⁺, CD11c⁺) (FIG. 16G), the fraction of activated DCs (CD80⁺, CD86⁺) (FIG. 16H), total CD4⁺ (FIG. 16I), CD8⁺ (FIG. 16J) T cells and their early counterparts (CD69⁺), fully (CD44⁺, CD62L⁻) activated fractions, total macrophages (MHCII⁺, CD11b⁺) and their activated fractions (F4/80⁺)(FIG. 16K);

FIG. 17, supporting FIGS. 4A-4L, show persistent upregulation of PIC and T1RG after transient TTFields treatment in KR158-luc GBM cells. FIG. 17 provides exemplary bar graphs showing that the PIC IL6 and T1IRG ISG15 remained upregulated in response to TTFields in a STING/AIM2-dependent manner for at least 3 days after TTFields cessation;

FIGS. 18A-18J, supporting FIGS. 4A-4L, show induction of anti-tumor immunity by TTFields requires STING and AIM2. FIGS. 18A-18J provide exemplary combo box and whisker and dot plots showing immunophenotyping of animals at 2 weeks after being immunized in various conditions as in FIG. 4, and rechallenged with KR158-luc GBM cells for CD4 and CD8 T cells, their fully (CD44+, CD62L⁻) and early (CD69+) activated counterparts, MDSCs (CD11b⁺/Ly6g/Ly6c⁺), and macrophages (MHCII⁺, CD11b⁺) in draining dcLNs at 2 weeks post immunization (FIGS. 18A-18C); for DCs, MDSCs, macrophages, CD4 and CD8 T cells and their fully and early activated counterparts in PBMCs at 2 weeks post primary immunization (FIGS. 18D-18F); for MDSCs, macrophages, CD4 and CD8 T cells and their fully activated counterparts in splenocytes at 2 weeks post immunization (FIGS. 18G-18H); and for early activated CD4 and CD8 T cells in PBMCs at 1 (FIG. 18I) and 2 weeks (FIG. 18J) post rechallenge;

FIGS. 19A-19C, supporting FIG. 5B, show general immune cell markers for different immune cell type and subtypes in single PBMCs by scRNA-seq in 12 GBM patients. FIGS. 19A-19C provide exemplary heatmaps of expression of indicated general immune cell marker genes in single PBMCs by scRNA-seq in 12 GBM patients at the single cell level showing their expression distribution across all clusters in the UMAP graph at Resolution 1;

FIGS. 20A-20D, supporting FIG. 5B, show markers for non-lymphocytes by scRNA-seq in 12 GBM patients. FIGS. 20A-20D provide exemplary heatmaps of expression of indicated marker genes for lymphocytes assessed by scRNA-seq in 12 GBM patients for total T cells (FIG. 20A), CD4 T cells (FIG. 20B), CD8 T cells (FIG. 20C), and B cells (FIG. 20D) at the single cell level showing their expression distribution across all clusters in the UMAP graph at Resolution 1;

FIGS. 21A-21E, supporting FIG. 5B, show markers for non-lymphocytes by scRNA-SEQ in 12 GBM patients. FIGS. 21A-21E provide exemplary heatmaps of expression of indicated marker genes for non-lymphocytes assessed by scRNA-seq in 12 GBM patients for DCs (FIG. 21A), NK cells (FIG. 21B), Monocytes (FIG. 21C), Megakaryocyte/platelets (FIG. 21D), and Hematopoietic stem cells (FIG. 21E) at the single cell level showing their expression distribution across all clusters in the UMAP graph at Resolution 1;

FIGS. 22A-22D, supporting FIG. 5B, show markers for Clusters 31 (plasmacytoid DCs or PDCs, 25 (classical DCs or cDCs), 17 (T1iRG monocytes), and 22 (NK cells) by scRNA in 12 GBM patients. FIGS. 22A-22D provide exemplary heatmaps of expression of the indicated marker genes assessed by scRNA-seq in 12 GBM patients for Cluster 31 (plasmacytoid DCs) (FIG. 22A), Cluster 25 (cDCs) (FIG. 22B), Cluster 17 (T1IRG classical monocytes) (FIG. 22C), and Cluster 22 (Xc11/2⁺, Klrc1+NK cells) (FIG. 22D) at the single cell level showing their expression distribution across all clusters in the UMAP graph at Resolution 1;

FIGS. 23A-23D, supporting FIGS. 5B-5C, show markers for Clusters 0 (Cytotoxic effector T cells), 9 (exhausted CD8), 6 (transitional memory CD8), and 26 (memory CD8) by scRNA-seq in 12 GBM patients. FIGS. 23A-23D provide exemplary heatmaps of expression of indicated marker genes assessed by scRNA-seq in 12 GBM patients for Cluster 0 (cytotoxic effector T cells) (FIG. 23A), Cluster 9 (exhausted effector CD8 T cells) (FIG. 23B), Cluster 6 (transitional memory CD8 T cells) (FIG. 23C), and Cluster 26 (memory CD8 T cells) (FIG. 23D) at the single cell level showing their expression distribution across all clusters in the UMAP graph at Resolution 1;

FIGS. 24A-24D, supporting FIG. 5D, show UMAP graph for each patient before and after TTFields overlay UMAP of PBMCs in 12 GBM patients. FIGS. 24A-24D provide an exemplary UMAP graph for each patient pre and post TTFields and overlay UMAP of PBMCs in 12 GBM patients for Separate pre-TTF and post-TTF UMAP graphs of individual patients (FIGS. 24A-24C) and combined pre-TTF, combined post-TTF, and overlap of combined pre- and post-TTF UMAP graphs (FIG. 24D);

FIGS. 25A-25K, supporting FIGS. 5F-5P, show TTFields effects at the all-genes level on various immune cell clusters in PBMCs of GBM patients. FIGS. 25A-25K provide exemplary heatmaps of gene expression showing log FC of post-TTF expression of all-genes compared to pre-TTF expression of all genes in indicated PBMCs cell clusters in GBM patients with detectable pre- and post TTFields counts in the respective cell clusters;

FIGS. 26A-26M, supporting FIGS. 5F-5P, shows TTFields effects at the all-pathways level on various immune cell clusters in PBMCs of GBM patients. FIGS. 26A-26M provide exemplary heatmaps of gene expression showing log FC of post-TTF expression of all pathways of various immune cell clusters in PBMCs of GBM patients compared to pre-TTF expression of all pathways in indicated cell clusters in patients with detectable pre- and post TTFields counts in the respective cell clusters;

FIGS. 27A-27B, supporting FIGS. 5M-5P, show post TTFields activation status in pDCs and cDCs in GBM patients. FIGS. 27A-27B provide exemplary heatmaps of gene expression showing log FC of post-TTFields expression compared to pre-TTFields expression of 10 functionally critical pathways in pDCs (C31) (FIG. 27A) and cDCs (C25) (FIG. 27B) showing post-TTFields activation of T1IFN and T1IRG pathways and DC-critical pathways;

FIGS. 28A-28C, supporting FIGS. 5I-5J, show GSEA showing activation of function critical pathways in cytotoxic effectors (C0), transitional memory (C6), and memory CD8 T cells (C26) in PBMCs of GBM patients. FIGS. 28A-28C provide exemplary graphs of GSEA of functionally critical pathways showing their post-TTFields enrichment or activation in Cytotoxic effectors (C0) (FIG. 28A), transitional memory (C6) (FIG. 28B), and memory CD8 T cells (C26) (FIG. 28C) in PBMCs of GBM patients;

FIG. 29, supporting FIG. 6A, shows Clonal TCRβ structures of all patients. FIG. 29 provides exemplary pie charts detailing TCRβ clonal structures and sequences in pre- and post-TTFields samples showing specific clonal expansion in 9 of 12 patients;

FIGS. 30A-30B, supporting FIG. 6A, show cumulative frequencies of detectable TCRβ clones in all patients. FIGS. 30A-30B provide exemplary line graphs of cumulative frequencies of detectable TCRβ clones in all patients pre and post TTFields showing clonal frequency expansion in the most abundant clones in post-TTFields T cells to different extents in all except for P12;

FIG. 31A, supporting FIGS. 6A-6B, provides an exemplary dot plot of log FC of the Simpson Diversity Index (DI) of TCRα showing TCRα clonal expansion after TTFields treatment (negative DI log FC) in 9 of 12 patients; and

FIGS. 31A-31B, supporting FIGS. 6A-6B, show TCRα clonal expansion correlates with TTFields treatment in GBM patients. FIGS. 31A-31B provide exemplary 2D area charts of the 200 most abundant TCRα clones in post TTFields T cells as compared to their proportions in pre-TTFields T cells showing clonal expansion in all 12 patients.

### DETAILED DESCRIPTION

The above and other aspects, features and other advantages of the present disclosure will be more clearly understood from the following detailed description taken in conjunction with the accompanying drawings.

Since becoming the new standard treatment for newly diagnosed GBM (5) and malignant pleural mesothelioma (6), and currently under late stage clinical investigation in several other solid tumors, a recurrent clinical observation has emerged among TTFields responders with GBM, in which a transient period of increased tumor contrast enhancement and edema often occur shortly after treatment initiation followed by a delayed objective radiographic response (7-10). In murine models of lung, colon, renal and ovarian cancers, TTFields were demonstrated to induce immunogenic cell death and promote recruitment of immune cells (11,12), thus raising hope that TTFields may provide a needed stimulus to reverse local and systemic immunosuppression in GBM patients. However, the molecular mechanism remains unclear and clinical evidence is lacking.

Checkpoint proteins function as inhibitors of the immune system (e.g., T-cell proliferation and IL-2 production) which can lead to dampening of the immune response. See, e.g., Azoury et al., Curr Cancer Drug Targets. 2015; 15(6):452-62. Checkpoint proteins can have a deleterious effect with respect to cancer by shutting down the immune response. Blocking the function of checkpoint proteins can be used to activate dormant T-cells to attack cancer cells. Checkpoint inhibitors are cancer drugs that inhibit checkpoint proteins in order to recruit the immune system to attack cancer cells.

Thus, there is an interest in using checkpoint inhibitors as a cancer treatment to block the activity of checkpoint proteins, enabling the production of cytokines and recruitment of tumor-specific T cells to attack cancerous cells and are an active area in immunotherapy drug development. As described herein, TTFields activate the immune system, in part, by triggering “danger” signals resulting from TTFields-induced mitotic disruptions as detected by DNA sensors. Activation of the immune system can create an environment where tumor cells are more susceptible to treatment with anti-cancer drugs such as checkpoint inhibitors or chemotherapy. However, determining when the immune system has been activated following exposure of cells or tissues to TTFields can be important to maximize the effects of such anti-cancer drugs.

For example, it would be beneficial to determine if additional exposure to TTFields would be advantageous to maximizing immune system activation in a given patient prior to treatment with anti-cancer drugs. If the patient's immune system is fully activated prior to administering an anti-cancer drug, the combination of the patient's innate immune system defenses and the activity of the anti-cancer drug can be maximized resulting in more efficacious treatment and/or reduction in the dose of the anti-cancer drug to minimize side effects.

In some instances, a patient can be exposed to TTFields for a period of time and assessed to determine if the TTFields exposure has activated the patient's immune system. If TTFields exposure has activated the immune system, anti-cancer therapy can be administered. If not, additional exposure to TTFields may be applied prior to treatment with anti-cancer drugs.

A biomarker such as a gene signature can be used to determine whether an individual patient's immune system has been activated following exposure to TTFields. The term “gene signature,” as used herein, refers to an expression pattern of one or more genes or gene clusters that display differential expression indicative of a biological or other condition. A gene signature can be measured, for example, by determining the expression level of one or more genes that are part of the gene signature before and after a treatment with a drug or device or an environmental condition. The change in the expression level of the one or more genes can be indicative of a biological change that can be used to determine an optimal treatment.

As described herein, the expression patterns exhibited by a gene signature associated with activation of the immune system following exposure to TTFields can be used to determine if a patient or subject's immune system has been activated. If the subject or patient's immune system has been activated, anti-cancer therapy (e.g., treatment with a checkpoint inhibitor, chemotherapy, or other treatment) can be administered to the subject or patient. If the patient's immune system has not been activated, TTFields treatment can be continued or another course of action can be taken to treat the subject or patient (e.g., combine TTFields with another anti-cancer therapy).

Aspects described herein provide methods of treating a subject with a checkpoint inhibitor by:

(a) determining a first expression level of nucleic acids expressing cytokines and cytotoxic genes in immune T cells of the subject;
(b) determining a first expression level of nucleic acids expressing T cell functional regulators in immune T cells of the subject;
(c) determining a first expression level of nucleic acids expressing naïve T cell markers in immune T cells of the subject;
(d) determining a first expression level of nucleic acids expressing regulatory T cell factors in immune T cells of the subject;
(e) determining a first expression level of nucleic acids expressing immune inhibitory receptors in immune T cells of the subject;
(f) determining a first expression level of nucleic acids expressing type 1 interferon response genes in immune T cells of the subject;
(g) applying alternating electric fields to tumor cells at a frequency between 100 and 500 kHz after steps a-f and prior to steps h-m;
(h) determining a second expression level of nucleic acids expressing cytokines and cytotoxic genes in immune T cells of the subject;
(i) determining a second expression level of nucleic acids expressing T cell functional regulators in immune T cells of the subject;
(j) determining a second expression level of nucleic acids expressing naïve T cell markers in immune T cells of the subject;
(k) determining a second expression level of nucleic acids expressing regulatory T cell factors in immune T cells of the subject;
(l) determining a second expression level of nucleic acids expressing immune inhibitory receptors in immune T cells of the subject;
(m) determining a second expression level of nucleic acids expressing type 1 interferon response genes in immune T cells of the subject; and
(n) treating the subject with a checkpoint inhibitor if (i) the first expression level of at least 50% of the nucleic acids expressing cytokines and cytotoxic genes is lower than the second expression level of nucleic acids expressing cytokines and cytotoxic genes, (ii) the first expression level of at least 50% of the nucleic acids expressing T cell functional regulators is lower than the second expression level of nucleic acids expressing T cell functional regulators, (iii) the first expression level of at least 50% of the nucleic acids expressing naïve T cell markers is greater than the second expression level of nucleic acids expressing naïve T cell markers, (iv) the first expression level of at least 50% of the nucleic acids expressing regulatory T cell factors is greater than the second expression level of nucleic acids expressing regulatory T cell factors, (v) the first expression level of at least 50% of the nucleic acids expressing immune inhibitory receptors is either greater than or unchanged compared to the second expression level of nucleic acids expressing immune inhibitory receptors, and (vi) the first expression level of nucleic acids expressing type 1 interferon response genes is either greater than or unchanged compared to the second expression level of nucleic acids expressing type 1 interferon response genes.

In some instances, the nucleic acids expressing cytokines and cytotoxic genes are selected from the group consisting of GZMB, GZMH, GZMK, GNLY, PRF1, INFG, NKG7, CX3CR1, CCL3, and CCL4.

In some instances, the nucleic acids expressing T cell functional regulators are selected from the group consisting of ZEB2, ZHF683, HOPX, TBX21, ID2, TOX, GF11, EOMES, and HMGB3.

In some instances, the nucleic acids expressing naïve T cell markers are selected from the group consisting of TCF7, SELL, LEF1, CCR7, and IL7R.

In some instances, the nucleic acids expressing regulatory T cell factors are selected from the group consisting of IL2RA, FOXP3, and IKZF2.

In some instances, the nucleic acids expressing immune inhibitory receptors are selected from the group consisting of LAG3, TIGIT, PDCD1, and CTLA4.

In some instances, the nucleic acids expressing type 1 interferon response genes are selected from the group consisting of ISG15, ISG20, IL32, IFI44L, and IFITM1.

In some instances, the nucleic acids comprise one or more of GZMB, GZMH, GZMK, GNLY, PRF1, INFG, NKG7, CX3CR1, CCL3, CCL4, ZEB2, ZHF683, HOPX, TBX21, ID2, TOX, GF11, EOMES, HMGB3, TCF7, SELL, LEF1, CCR7, IL7R, IL2RA, FOXP3, IKZF2, LAG3, TIGIT, PDCD1, CTLA4, ISG15, ISG20, IL32, IFI44L, and IFITM1 (“Gene Signature”). NCBI (National Center for Biotechnology Information) Reference Numbers and nucleic acid sequences for the nucleic acids of this Gene Signature (including variants thereof) and sequences for the proteins encoded by the nucleic acids can be found in Table 7 and at www.ncbi.nlm.nih.gov/refseq/. Alterations in the expression levels of one or more of the genes in the Gene Signature, as described in FIGS. 5 and 6 and the accompanying text herein, can be used to determine if a subject or patient has an activated immune system after exposure to alternating electric fields.

In some instances, the checkpoint inhibitor is selected from the group consisting of ipilimumab, pembrolizumab, nivolumab, cemilimab, atezolimumab, avelumab, durvalumab, IDO1 inhibitors (e.g., BMS-986205, epacadostat, indoximod, KHK2455, SHR9146), TIGIT inhibitors (e.g., MK-7684, etigilimab, tiragolumab, BMS-986207, AB-154, ASP-8374), LAG-3 inhibitors (e.g., eftilagimod alpha, relatlimab, LAG525, MK-4280, REGN3767, TSR-033, BI754111, Sym022, FS118, MGD013), TIM-3 inhibitors (e.g., TSR-022, MBG453, Sym023, INCAGN2390, LY3321367, BMS-986258, SHR-1702, RO7121661), VISTA inhibitors (e.g., JNJ-61610588, CA-170), and B7-H3 inhibitors (e.g., enoblituzumab, MGD009, omburtamab).

In some instances, the cells are selected from the group consisting of brain cells, blood cells, breast cells, pancreatic cells, ovarian cells, lung cells, and mesenchymal cells. In some instances, the cells are brain cells. In some instances, the cells are cancer cells.

Further aspects, provide kits comprising nucleic acids for detecting nucleic acids expressing cytokines and cytotoxic genes, nucleic acids expressing T cell functional regulators, nucleic acids expressing naïve T cell markers, nucleic acids expressing regulatory T cell factors, nucleic acids expressing immune inhibitory receptors, and nucleic acids expressing type 1 interferon response genes.

In one embodiment, the nucleic acids expressing cytokines and cytotoxic genes are selected from the group consisting of GZMB, GZMH, GZMK, GNLY, PRF1, INFG, NKG7, CX3CR1, CCL3, CCL4.

In another embodiment, the nucleic acids expressing T cell functional regulators are selected from the group consisting of ZEB2, ZHF683, HOPX, TBX21, ID2, TOX, GF11, EOMES, and HMGB3.

In a further embodiment, the nucleic acids expressing naïve T cell markers are selected from the group consisting of TCF7, SELL, LEF1, CCR7, and IL7R.

In one embodiment, the nucleic acids expressing regulator T cell factors are selected from the group consisting of IL2RA, FOXP3, and IKZF2.

In another embodiment, the nucleic acids expressing immune inhibitory receptors are selected from the group consisting of LAG3, TIGIT, PDCD1, and CTLA4.

In a further embodiment, the nucleic acids expressing type 1 interferon response genes are selected from the group consisting of ISG15, ISG20, IL32, IFI44L, and IFITM1.

In yet another embodiment, the nucleic acids comprise one or more of GZMB, GZMH, GZMK, GNLY, PRF1, INFG, NKG7, CX3CR1, CCL3, CCL4, ZEB2, ZHF683, HOPX, TBX21, ID2, TOX, GF11, EOMES, HMGB3, TCF7, SELL, LEF1, CCR7, IL7R, IL2RA, FOXP3, IKZF2, LAG3, TIGIT, PDCD1, CTLA4, ISG15, ISG20, IL32, IFI44L, and IFITM1. NCBI (National Center for Biotechnology Information) Reference Numbers and nucleic acid sequences for the nucleic acids of this Gene Signature (including variants thereof) and sequences for the proteins encoded by the nucleic acids can be found in Table 7 and at www.ncbi.nlm.nih.gov/refseq/. Alterations in the expression levels of one or more of the genes in the Gene Signature, as described in FIGS. 5 and 6 and the accompanying text herein, can be used to determine if a subject or patient has an activated immune system after exposure to alternating electric fields.

An exemplary kit can comprise nucleic acid probes directed to one or more genes of the Gene Signature for use in an assay to measure a genes expression level before and after exposure to alternating electric fields along with reagents and apparatus for measuring gene expression levels, as described herein. The nucleic acid probes can be single or double-stranded DNA or RNA, labeled or unlabeled, or synthesized or naturally occurring.

The non-limiting examples below illustrate how to make and use aspects described herein and provide additional supporting data, with reference to the Figures, for the embodiments and aspects described herein, including modifications and alternations. Without being bound by any theories or hypotheses, the examples may include possible explanations for the described data. Accordingly, it is intended that the present invention not be limited to the examples provided below, but that it has the full scope defined by the language of the claims listed below, and equivalents thereof.

### EXAMPLES

### Example 1—TTFields Induce Formation of Cytosolic Micronuclei Clusters that Recruit cGAS and AIM2

A potential link between TTFields and immune activation is cytosolic micronuclei created by TTFields-induced mitotic disruptions^{13, 14}. As previously reported, small free-standing cytosolic micronuclei were detected by DAPI counter-staining after 24 hour treatment with TTFields (at 200 kHz, unless otherwise noted) in 3 human GBM cell lines: U87MG¹⁵, LN428¹⁶, and LN827¹⁷. However, large clusters of micronuclei extending directly from the nucleus through a narrow bridge were also found, almost exclusively, in many TTFields-treated cells (FIGS. 1A-1B, quantified in FIG. 1G; wide field in FIG. 7).

In non-pathogenic conditions, cytosolic free DNA signifies aberrant host DNA metabolism and is recognized by DNA sensors including cGAS¹⁸⁻²⁰ and AIM2²¹⁻²³, triggering strong “danger” signals in innate immune responses in several types of cancer^{24, 25, 26-28}. Therefore, experiments were conducted to assess whether cGAS and AIM2 are recruited to these large cytosolic micronuclei clusters. Both DNA sensors were densely concentrated in all the micronuclei clusters identified (FIGS. 1A-1F; FIG. 7), indicating that these clusters are unshielded by the nuclear envelope from being detected. Importantly, a redistribution of cGAS and AIM2 from a scattered cytosolic pattern to the perinuclear region in TTFields-treated cells (FIGS. 1A-1F) was observed, even in those without micronuclei clusters (FIG. 8), raising the possibility that the nuclear envelope may be impaired in these cells.

To assess the integrity of the nuclear envelope under TTFields, the distribution of LAMINs A and C (LAMINA/C), 2 major structural proteins lining the inside of the nuclear envelope was determined^{29, 30}. As predicted, in all 3 GBM cell lines, exposure to TTFields resulted in disorganization of LAMINA/C at the site of micronuclei cluster protrusions (FIG. 1E, FIG. 9, FIG. 10C, FIGS. 10C and 10G). In contrast, isolated cytosolic micronuclei and occasional fragmented nuclei present in these cells were independent of TTFields treatment, protected by a LAMINA/C-based membrane, and as a result did not recruit cGAS and AIM2 (FIGS. 11A-11D). Moreover, most of the affected cells were not in metaphase when TTFields cause spindle disruption^{13, 14}, leading to the question of whether cell cycle entry is necessary for TTFields effects on the nuclear envelope.

To address this question, cells were pretreated for 24 hours prior to and during a 24 hour exposure to TTFields with ribociclib, a potent inhibitor of cyclin-dependent kinases 4 and 6, to induce G₁ arrest³¹ (FIG. 1H). In all 3 lines, the percentage of cells with cGAS and AIM2-recruited micronuclei clusters after TTFields consistently decreased in G₁-arrested cells compared to cycling cells (FIGS. 1E-1F; FIGS. 10C, 10D, 10G, 10H). Ribociclib treatment alone did not increase cluster formation (FIG. 1C; FIGS. 10A, 10B, 10E, 10F). These results indicate that S phase entry may be necessary for TTFields-induced nuclear envelope disruption and micronuclei formation.

As TTFields are at an advanced stage of clinical testing in various solid tumors, the effect of TTFields treatment on the human lung and pancreatic adenocarcinoma cell lines A549³² and PANC-1³³, respectively, was examined Cytosolic micronuclei clusters with intense cGAS and AIM2 recruitment were similarly observed in these cells after 24 hour exposure to TTFields at 150 kHz, a previously defined optimal frequency for these cancers^{34, 35} (FIGS. 12A and 12C).

Overall, TTFields generate cytosolic naked micronuclei clusters in GBM and other cancer cell types through disruption of the nuclear envelope, thereby recruiting 2 major cytosolic DNA sensors cGAS and AIM2 to create a ripe condition for activation of their cognate inflammasomes.

### Example 2—TTFields Activate the cGAS-STING and AIM2-Caspase-1 Inflammasomes

STING, a signaling scaffold downstream of cGAS, recruits and activates TANK-binding serine/threonine kinase 1 (TBK1) to phosphorylate interferon regulatory factor 3 at S396 (pS396-IRF3) and the canonical NFkB complex component p65 at 5536 (0536-p65)¹⁸⁻²⁰, which then migrate to the nucleus to upregulate PIC, T1IFN and T1IFN response genes (T1IRGs)¹⁸⁻²⁰. In response to TTFields, p5396-IRF3 level increased in all 3 GBM cell lines as compared to non-treated cells, as did pS536-p65 level in LN827 and U87MG cells (FIGS. 2A-2B). In LN428 cells, despite having higher basal STING expression, pS536-p65 levels slightly decreased after TTFields, which corresponded to rapid STING downregulation, likely reflecting higher, not lower, STING activation, resulting in its own accelerated degradation (FIGS. 2A-2B; FIGS. 13A-13C), as previously reported^{36, 37}.

Indeed, LN428 cells exhibited more robust TTFields-induced cGAS recruitment to micronuclei clusters (FIG. 1G), compared to U87MG and LN827 cells. In all 3 lines, a higher concentration of p5396-IRF3 and p65 in and around the micronuclei clusters in response to TTFields (FIG. 2C; FIGS. 13B-13C) was detected, followed by upregulation of PICs (FIG. 2D; FIG. 14D), T1IFNs and T1IRGs (FIGS. 2E, 2G; FIG. 14B) that peaked around 72 hours (FIG. 14A) and stringently depended on the presence of STING (FIGS. 2F-2G; FIGS. 14C-14E). Similar responses to TTFields were also observed in A549 and PANC-1 cell lines (FIGS. 12B and 12D). Thus, TTFields activate the cGAS-STING inflammasome in GBM and other cancer cell types, leading to increased production of PICs and T1IFNs.

Next, to determine if TTFields activate the AIM2-caspase-1 inflammasome in an AIM2-dependent manner, the proportion of cells with or without AIM2 depletion that expressed activated caspase 1, a key AIM2 target, after TTFields was measured, utilizing FAM-YVAD-FMK (SEQ ID NO: 47), a fluorescently labeled specific irreversible inhibitor of activated caspase 1. A new right-shifted fluorescent peak was consistently detected only in the scrambled shRNA control treated with TTFields, but not in AIM2-depleted cells (FIG. 3A; FIG. 15A).

Activated caspase 1 regulates proteolytic cleavage and release of PICs and the membrane pore-forming GASDERMIN D (GSDMD)³⁸, an executor of highly immunogenic programmed necrotic cell death. There was a 2.5-3.5-fold increase in the fraction of proteolytic N-terminal cleavage product of GSDMD in TTFields-treated U87MG and LN827 cells with intact AIM2 (FIG. 3B). AIM2 was not detectable by immunoblotting in LN428 cells under the same condition. Yet in all 3 lines, there was a 2.5 to 3-fold AIM2-dependent increase in release of cytoplasmic lactate dehydrogenase (LDH)²¹⁻²³ into the supernatants after 24 hours of TTFields treatment, confirming TTFields-induced necrotic cell death (FIG. 3C). The increased LDH release was not due to secondary necrosis occurring in late apoptosis^{39, 40} since apoptosis caused by the GBM standard cytotoxic drug temozolomide (TMZ) either alone or in combination with TTFields did not increase LDH release above those observed in the non-treated and TTFields-treated cells, respectively (FIG. 15B).

In summary, cytosolic micronuclei clusters produced by TTFields recruit cGAS and AIM2 and activate their cognate inflammasomes leading to upregulation of PICs and T1IFNs.

### Example 3—TTFields-Treated GBM Cells Provide an Immunizing Platform Against GBM

Next, a C57BL/6J syngeneic GBM model that is clinicopathologically similar to human GBM including rapid intracranial growth, poor immunogenicity, and resistance to immunotherapy was used⁴¹⁻⁴³. The cGAS-STING and AIM2-caspase-1 inflammasomes were activated by TTFields in luciferase-tagged KR158 cells (KR158-luc) in a STING (FIGS. 16A and 16C) and AIM2 (FIGS. 16B, 16D, and 16E) dependent manner, confirming that TTFields-induced activation of cytosolic DNA sensors and their cognate inflammasomes is conserved across cancer cell types and species.

To examine the effects of TTFields-induced PICs and T1IFNs on immune cells, conditioned media was collected from KR158-luc cells with or without STING (ST) or AIM2 (A) knockdown or double knockdown (DKD) that were either non-treated or TTFields-treated to culture splenocytes isolated from healthy 6-8 weeks-old C57BL/6J mice for 3 days. The fractions of T cells, DCs, and macrophages were determined (FIG. 16F). Total and activated (CD80/CD86±) DCs and the early activated (CD69+)⁴⁴⁴⁵ and effector (CD44^{high}/CD62L^{low})^{46, 47} fractions of CD4 and CD8 T cells increased with conditioned media from TTFields-treated KR158-luc when either STING or AIM2 was present compared to media from non-treated cells (FIGS. 16G-16J). Similar trends were also observed in total and activated macrophages, but to a lesser degree (FIG. 16K). Thus, PICs and T1IFNs induced by TTFields require both STING and AIM2 and provide a potential link between TTFields and the adaptive immune system.

In one aspect, TTFields-treated GBM cells can be harnessed to induce adaptive immunity against GBM tumors. KR158-luc cells were exposed to TTFields for 72 hours initially before stereotactically implanting the cells into the posterior right frontal cerebrum of C57BL/6J mice to provide both immunogens and adjuvant signals, while avoiding confounding effects of TTFields on tumor stromal and immune cells (FIG. 4A). Importantly, it was confirmed that STING and AIM2-dependent upregulation of PICs, T1IFNs and T1IRGs in KR158-luc cells persisted for at least 3 days after TTFields cessation, providing the rationale for their use as an immunizing vehicle (FIG. 17).

Vaccinated animals were immunophenotyped and their brains examined histologically 2 weeks after implantation or monitored for tumor growth by bioluminescence imaging (BLI) and overall survival (OS). To test for an anti-tumor memory response, surviving animals were re-challenged at day 100, and compared to the same number of vaccine-naïve, sex-matched, 6-8 weeks old C57BL/6J controls with a 2-fold higher number of non-treated KR158-luc cells with respect to immune responses and OS.

At day 7 (D7) post implantation, all groups developed comparable BLI signals, confirming that primary tumor establishment was equivalent in all conditions. Subsequently, however, all but 1 animal (38 of 39 or 97%) in the 3 control groups, i.e., scrambled shRNA/non-treated (Sc), STING-AIM2 DKD/TTFields-treated (DKD-TTF), and STING-AIM2 DKD/non-treated (DKD) developed progressive brain tumors and succumbed by day 100 with median OS (mOS) of 45 days.

In contrast, 10 of 15 (66%) animals receiving scrambled shRNA/TTFields-treated cells (Sc-TTF) had no detectable tumor at day 100 with mOS not reached (FIGS. 4B, 4D and 4E). When these 10 surviving Sc-TTF animals were re-challenged with 2-fold parental KR158-luc cells, 6 (60%) survived for at least 144 more days without any detectable tumor, as compared to none of the 12 naïve controls surviving past 45 days with mOS of only 38 days despite their being much younger in age (FIGS. 4C-4E).

The 4 Sc-TTF mice that succumbed by 100 days still exhibited a significant delay in tumor growth and improved survival compared to the naïve controls. In summary, 40% (6 of 15) animals immunized with Sc-TTF cells developed robust anti-tumor immunity and another 25% (4 of 15) derived partial immunity in a TTFields, STING and AIM2-dependent manner—a remarkable feat for KR158, a poorly immunogenic model that closely resembles human GBM.

To define the immunological basis of these positive clinical observations, the ipsilateral deep cervical lymph nodes (dcLNs), thought to directly drain the brain and the ipsilateral head and neck⁴⁸⁻⁵⁰, were harvested for immunophenotyping. Compared to animals receiving Sc cells, the fraction of DCs in dcLNs increased in mice immunized with Sc-TTF cells, which was reversed when DKD-TTF cells were injected. DKD cells resulted in no difference in DCs in dcLNs compared to Sc cells (FIG. 4F), indicating that STING and AIM2 only became dominant with TTFields. Importantly, of the DCs in dcLNs, the fraction of activated DCs (CD80/CD86±) doubled when Sc-TTF cells were implanted instead of Sc, DKD-TTF or DKD cells (FIG. 4F), which coincided with an increase in the fractions of early activated CD69⁺ CD4 and CD8 T cells, even though the total and activated CD4 and CD8 fractions had not increased yet by this time (FIGS. 18A-18B).

Next, the peripheral immune compartment was examined for the emergence of a memory adaptive response to KR158 tumors by temporally immunophenotyping splenocytes and peripheral blood mononuclear cells (PBMCs) at Week 2 post primary immunization and then at Week 1 and 2 post re-challenge, with minimal changes expected at the earlier time point. At Week 2 post immunization, as predicted, there was only a trend of increase in DCs, and no change in lymphocytes in PBMCs except that CD69⁺ CD8 T cells was higher in Sc-TTF animals (FIGS. 18D-18F).

Surprisingly, however, an increase in total and activated DCs and CD69⁺ CD8 T cells was detected in splenocytes from Sc-TTF animals, compared to controls (FIG. 4G; FIG. 18H), attesting to the strength of TTFields-induced immune stimulation. Indeed, infiltration of T (CD3) and CD8 T (CD3⁺CD8⁺) cells in Sc-TTF tumors increased compared to other control tumors (FIG. 4H). Upon re-challenge, the fractions of DCs and activated CD4 and CD8 T cells rapidly increased at Week 1 and rose further at Week 2 (FIGS. 4I-4J), while those of CD69⁺ CD4 and CD8 T cells increased only at Week 1, not Week 2 (FIGS. 18I-18J), in the rechallenged Sc-TTF cohort as compared to the vaccine naïve controls. Of note, no differences in myeloid derived suppressive cells (CD11b⁺/Ly6g/Ly6c⁺) and macrophages were detected in the different cohorts at any time (FIGS. 18C, 18D and 18G).

To confirm the presence of central memory (CM) T cells in the 6 long-term surviving rechallenged Sc-TTF mice, the fractions of CM (CD44⁺CD62L⁺) CD4 and CD8 T cells⁵¹⁻⁵⁴ in their dcLNs and spleens at 20 weeks post rechallenge was measured. For control mice, the same number of KR158-luc cells were implanted into an age- and sex-matched cohort of 6 naïve mice and their dcLNs and spleens were analyzed 2 weeks later. The fractions of CM and effector (CD44⁺CD62L⁻)⁵¹⁻⁵⁴ T cells were consistently higher in Sc-TTF mice than in the naïve controls (FIGS. 4K-4L).

In summary, TTFields vigorously activate the cGAS-STING and AIM2-caspase-1 inflammasomes through cytosolic micronuclei cluster formation, thereby providing complete “danger” signals to generate anti-tumor immunity against poorly immunogenic tumors like GBM.

### Example 4—Gene Signature Reflecting Adaptive Immune Activation by TTFields in GBM Patients Via a T1IRG-Based Trajectory

The observations in the KR158 model led to the hypothesis that TTFields similarly activate adaptive immunity in patients with GBM, specifically through a T1IRG-based trajectory, and that a gene signature linking TTFields to adaptive immunity is identifiable. To that end, PBMCs were collected from 12 adult patients with newly diagnosed GBM after completing chemoradiation at the following 2 times—within 2 weeks before and about 4 weeks after initiation of TTFields and TMZ (FIG. 5A)—to perform 1) single-cell RNA-seq (scRNA-seq) to identify the cell types and subtypes responsible for TTFields effects; and 2) deep bulk RNA-seq of isolated T cells to identify a gene signature that captures broad effects of TTFields-induced T1IFNs across T cell subtypes. The high sequencing depth also enabled a focused clonal analysis of the most abundant T cell receptor (TCR) clones to provide direct evidence of adaptive immune activation by TTFields. The baseline patient characteristics are shown in Table S1. Cell viability and sequencing data for scRNA-seq and bulk RNA-seq are shown in Tables S1-S3, respectively.

In total, 193,760 PBMCs were resolved in the 24 paired samples (Table S3). Using the graph-based cell clustering technique UMAP⁵⁵, the graph was partitioned using increasing resolution parameter values (0.1, 0.3, 1, 3, 5 and 10). Resolution 1 was chosen as it produced reasonably sized clusters, partitioning PBMCs into 38 biologically recognized subtypes of 8 main cell types (FIG. 5B; FIGS. 19A-23D). To more accurately annotate T cell clusters, a gene set containing cell type markers and functional regulators was assembled, gleaned from the UMAP clustering and literature review⁵⁶⁻⁵⁹ (FIG. 5C).

For instance, C15 contained naïve CD8 T cells, while C37 expressing granzyme K (GZMK) constituted transitional or partially activated CD8 T cells^{57, 69}. Cytotoxic effectors populated C0 and differed from exhausted effectors of C9 in that C0 expressed the cytotoxic regulator ZNF683^{61, 62} and lacked the inhibitory marker TIGIT and the regulatory T cell (T_{reg}) factor IKZF2⁶³ found in C9 (FIG. 5C; FIGS. 23A-23B). C6 and C26 comprised transitional and long-lived memory CD8 T cells, respectively, and are distinguished from each other by GZMK (C6), GZMB⁶⁴, CCL3⁶⁵ and CCR7^{66, 67} (C26) (FIG. 5C; FIGS. 23C-23D).

An overlay of the pre- and post-TTFields UMAP graphs revealed proportional increases in several clusters (FIG. 5D; FIGS. 27A-27B). Consistent with TTFields inducing the immune system via a T1IFN-based trajectory, higher proportions were found of plasmacytoid DCs (pDCs) (C31) (FIG. 5F), a specialized DC subtype that is both a direct target and the highest producer among DC subtypes of T1IFNs and key in linking the innate and adaptive immune systems⁶⁸⁻⁷⁰, and of a monocyte subtype (C17) expressing T1IRGs including IFI44L, MX1 and ISG15 (FIG. 5G). There was also a trend of increase in the XCL1/2⁺KLRC1⁺ subtype (C22) of NK cells, another major T1IFN-responsive innate immune cell type^{71, 72} (FIG. 5H).

To confirm that these 3 clusters constituted the front of the TTFields-induced T1IRG-based pathway trajectory, a global survey was conducted at the single cell level before and after TTFields for the mean expression of GO-0034340, a major T1IRG pathway with 99 genes annotated by Gene Ontology⁷³. Indeed, this T1IRG pathway formed an upregulated arc in response to TTFields that spanned these very 3 clusters and extended to other innate cell types, including non-classical monocytes (C8), classical NK cells (C1) and classical DCs or cDCs (C25) (FIG. 5E).

When gene coverage was expanded to all genes and pathways or cell-specific pathways using the gene set enrichment analysis (GSEA⁷⁴), there was widespread expression upregulation in pDCs in all 9 patients with detectable pre- and post-TTFields pDCs, specifically in T1IRG and DC-regulatory pathways (Table 5 and FIGS. 5M-5N; FIG. 26A; FIG. 27A). Moreover, post-TTFields pDCs upregulated the IFNg (T2IFN) pathway known to promote DC maturation⁷⁵ (Table 5). Although no numerical increase was observed, just as in the KR158 model where the increase was noted mostly in dcLNs, cDCs in 11 PBMCs exhibited pervasive post-TTFields upregulation of genes and pathways analogous to those in pDCs (FIGS. 5O-5P; FIG. 26B; FIG. 8B). Likewise, TTFields treatment led to global upregulation in C17 and C22 (FIG. 25A, FIG. 25D; FIG. 26C, FIG. 26F) and in other innate clusters, albeit with higher inter-patient variations (FIG. 25B, FIG. 25C, FIG. 25E; FIG. 26D, FIG. 26E, FIG. 26G). Taken together, these results confirmed robust gene upregulation in DCs and innate cells post TTFields in GBM patients, specifically following a T1IRG-based trajectory.

Next, whether effector T cells were activated following TTFields-induced DC activation, as observed in the KR158 model was considered. Although cytotoxic (C0) and exhausted (C9) effectors did not increase in proportion, their expression profiles and that of activated CD4 (C4) showed global gene upregulation post-TTFields to varying degrees across patients and clusters (FIGS. 5I-5J; FIGS. 25G-25H; FIG. 26I-26J). GSEA of C0 revealed enrichment in MHC-binding, NFkB⁷⁶, IL-1⁷⁷, and Toll-like-receptor-3^{78, 79} pathways (FIG. 28A) among others, which have been specifically implicated in antigen-specific CD8 effector activation and expansion. Of note, C0 cells also upregulated the Fas/FasL pathway (FIG. 28A), known to promote activation-induced cell death in cytotoxic effectors⁸⁰, presumably contributing to the lack of increase in C0 as they transition to memory T cells at 4 weeks after TTFields start.

Consistent with this notion, there was a trend of increase in long-lived memory CD8 T cells (C26), which coincided with a contraction in transitional memory CD8 T cells (C6) (FIGS. 5K-5L), with both exhibiting global upregulation across patients to varying degrees (FIGS. 25I-25J; FIGS. 26K-26L). GSEA of C26 and C6 showed enrichment in shared regulatory pathways previously implicated in memory T cells development and maintenance, including the mTOR^{81, 82} and complement activation⁸³⁻⁸⁵ pathways (FIGS. 28B-28C).

Peripheral TCR clonal expansion, a hallmark of adaptive immune activation^{86, 87}, was recently shown to have high concordance with tumor-infiltrating TCR clones in several cancers, especially for the most abundant clones^{88, 89}. Therefore, TCRab V(D)J sequences were extracted from the deep RNA-seq of T cells isolated from the same 12 PBMCs (Table 6) to determine if TTFields treatment resulted in TCR clonal expansion. TCR diversity was quantified using the Simpson's diversity index (DI), the average proportional abundance of TCR clones based on the weighted arithmetic mean—high and low values indicate even distribution and expansion, respectively, of TCR clones^{90, 91}.

Of the 12 patients, 9 exhibited negative log fold change (log FC) of TCRb DI after TTFields exposure, indicating clonal expansion (FIG. 6A; FIGS. 30A-30B). Notably, in all but 1 patient, the top 200 most frequent TCRb clones post TTFields, which accounted for 40 to 100% (median 67%) of detectable clones, showed substantial expansion compared to pre-TTFields T cells that inversely correlated with the DI (FIG. 6B; FIGS. 31A-31B). Similar expansion in 9 of 12 patients and uniform expansion (12 of 12) in the top 200 clones post TTFields were also observed in the TCRα VJ clonal analysis with the same patients at the 2 extremes of the DI scale and a slight change in ranks of those at or near the transition zone (FIG. FIGS. 30A-30B). Thus, TTFields exposure is associated with adaptive immune activation as reflected in clonal enrichment of peripheral T cells.

To corroborate that the observed TCR clonal expansion is more likely a tumor-specific response induced by TTFields than a non-specific reaction to the systemic inflammation created by TTFields-induced STING and AIM2 inflammasomes, the strength of correlation between TCRb clonal expansion and pDCs was measured. C31 proportions were moderately negatively correlated with TCRb DI log FC in the 9 patients with a full pDC dataset (Spearman coefficient r=−0.608, p=0.04) (FIG. 6C). To test if this correlation became stronger at the molecular level of pDC activation strength measured by gene expression log FC distribution, gene expression profiles of pDCs in these 9 patients were measured. The 3 patients with positive DI log FC (P12, P22 and P9) segregated into a distinct group with gene expression log FC more concentrated near 0, i.e., less disturbed, compared to the other 6 patients whose gene expression log FC values were more widely distributed, i.e., globally disturbed (FIG. 6D). A strong negative correlation between the Disturbance Score, defined as mean of absolute gene expression log FC across patients and the DI log FC (Spearman coefficient r=−0.8, p=0.014) was observed (FIG. 6E), indicating that the TCR clonal expansion was likely a direct result of TTFields inducing adaptive immunity via pDCs.

A gene signature of adaptive immune induction by TTFields was determined by taking advantage of the gene set used to annotate T cell clusters (FIG. 5C) to weigh against the TCRb DI log FC in all 12 patients (Table 7). DI log FC was negatively correlated with levels of cytokine, cytotoxic, and regulatory genes, and positively correlated with naïve and T_{reg} markers, suggesting that the lack of TCRb clonal expansion in the 3 patients with positive DI log FC may be due in part to increased T_{reg} activity. As expected, no correlation was observed between DI log FC and the 4 inhibitory receptors and T1IRGs examined, further arguing against the post-TTFields TCRa/b clonal expansion being a non-specific reaction to systemic inflammation.

TABLE 7
Gene Signature

| | | NCBI Ref Sequence |
|---|---|---|
| Gene | Type | Number mRNA |
| GZMB | cytokines and cytotoxic | NM_001346011.2 |
| | genes | |
| GZMH | cytokines and cytotoxic | NM_001270780.2 |
| | genes | |
| GZMK | cytokines and cytotoxic | NM_002104.3 |
| | genes | |
| GNLY | cytokines and cytotoxic | NM_001302758.2 |
| | genes | |
| PRF1 | cytokines and cytotoxic | NM_001083116.3 |
| | genes | |
| INFG | cytokines and cytotoxic | NM_000619.3 |
| | genes | |
| NKG7 | cytokines and cytotoxic | NM_001363693.2 |
| | genes | |
| CX3CR1 | cytokines and cytotoxic | NM_001171171.2 |
| | genes | |
| CCL3 | cytokines and cytotoxic | NM_002983.3 |
| | genes | |
| CCL4 | cytokines and cytotoxic | NM_002984.4 |
| | genes | |
| ZEB2 | T cell functional regulators | NM_001171653.2 |
| ZHF683 | T cell functional regulators | NM_001114759.3 |
| HOPX | T cell functional regulators | NM_001145459.2 |
| TBX21 | T cell functional regulators | NM_013351.2 |
| ID2 | T cell functional regulators | NM_002166.5 |
| TOX | T cell functional regulators | NM_014729.3 |
| GFI1 | T cell functional regulators | NM_001127215.3 |
| EOMES | T cell functional regulators | NM_001278182.2 |
| HMGB3 | T cell functional regulators | NM_001301228.2 |
| TCF7 | naïve T cell markers | NM_001134851.4 |
| SELL | naïve T cell markers | NM_000655.5 |
| LEF1 | naïve T cell markers | NM_001130713.3 |
| CCR7 | naïve T cell markers | NM_001301714.2 |
| IL7R | naïve T cell markers | NM_002185.5 |
| IL2RA | regulatory T cell factors | NM_000417.3 |
| FOXP3 | regulatory T cell factors | NM_001114377.2 |
| IKZF2 | regulatory T cell factors | NM_001079526.2 |

NCBI (National Center for Biotechnology Information) Reference Numbers and nucleic acid sequences for the nucleic acids of this Gene Signature (including variants thereof) and sequences for the proteins encoded by the nucleic acids can be found in Table 7 and at www.ncbi.nlm.nih.gov/refseq/.

Collectively, these results demonstrate that TTFields treatment leads to effective activation of adaptive immunity in patients with GBM, following the initial stimulation of immune cells that constitute the T1IFN pathways including pDCs and cDCs.

### Example 5—Discussion

With the recent recognition of a critical role for cytosolic DNA sensors' inflammasomes in stimulating anti-tumor immunity, the search for and development of pharmacological agonists of STING and AIM2 has dominated recent efforts in cancer immunotherapy⁹²⁻⁹⁹. To that end, these results place TTFields in a unique category of a dual activator of both inflammasomes through the formation of large clusters of cytosolic naked micronuclei. For brain tumors, the use of TTFields for this purpose has the added benefit of bypassing the blood brain barrier that often limits CNS delivery of pharmaceuticals. Equally important, this novel mechanism of action of TTFields may be generalizable and can be used for immunotherapy in other tumors, as shown in the lung cancer cell line A549 and the pancreatic cancer cell line PANC-1.

Although S phase entry was necessary for TTFields-induced micronuclei clusters (FIGS. 1A-1H; FIGS. 10A-10H), affected cells exhibited only focal rupture of the nuclear envelope and were not in M phase, suggesting that the TTFields-induced nuclear membrane disruption occurs most likely during the S and G₂ phases of the cell cycle. The nuclear envelope expands to accommodate increased DNA content by the end of the S phase and in the process becomes weakened in preparation for dissolution in prophase^{100, 101.} This process may be accentuated in cancer cells as their nuclear envelopes have been shown to be less stiff¹⁰², presumably rendering them more vulnerable to TTFields' effects. High-resolution microscopy with targeted arrest at key checkpoints will be necessary to determine the precise timing and nature of TTFields-induced nuclear disruption.

The robust activation of the cGAS-STING inflammasome components IRF3 and p65 in the large TTFields-induced cytosolic micronuclei clusters where cGAS is recruited and activated, instead of the true nucleus, and the subsequent substantial increases in PIC and T1IRG expression suggest that at least some of these large micronuclei clusters are transcriptionally active with PIC genes and T1IRGs present in them (FIGS. 1A-1H; FIGS. 2A-2G). Some degree of nuclear translocation of activated IRF3 and p65 cannot be ruled out, however, especially based on the observation that cGAS is redistributed to the perinuclear region in TTFields-treated cells with or without large cytosolic micronuclei clusters (FIG. 8), presumably due to TTFields-induced nuclear membrane disruption.

KR158 cells were pre-treated alone with TTFields prior to using them in immunization to avoid the confounding effect of TTFields on stromal cells in TiME. However, whether such effects impact, positively or negatively, the induction of anti-tumor immunity is unclear. TiME cells are predicted to exhibit similar responses to TTFields, including formation of micronuclei clusters that recruit and activate cGAS and AIM2, albeit likely less intense at 200 kHz, just as observed in other cancer cells (FIGS. 12A-12D) and also normal fibroblasts (data not shown). Although antagonistic effects from TTFields-exposed TiME cells cannot be ruled out, it is predicted that such antagonism, if present, is minor since in human patients with intracranial TTFields treatment, the link between TTFields and T1IRG-stimulated immune cells, e.g., pDCs, cDCs, monocytes and NK cells, was consistently observed in the 12 GBM patients examined (FIGS. 5A-5P; FIGS. 25A-25K, FIGS. 26A-26M, FIGS. 27A-27B).

The compelling TTFields-induced anti-tumor immunity observed in the KR158 model strongly argues for the adaptive immune activation observed in GBM patients after TTFields to be most likely a direct response to TTFields and not due to any potential lymphocytic homeostatic proliferation that might occur after TMZ-induced lymphopenia. The rebound phenomenon was noted for dose intense TMZ (i.e., 100 mg/m2 TMZ daily×21 days) and less so with standard dose TMZ (150 mg/m2 daily for 5 days) as employed in this trial, due to the former causing more severe lymphodepletion, which promotes steeper homeostatic proliferation^{103, 104.} Even so, it was noted that immunotherapy such as DC vaccination was more effective with a steeper homeostatic proliferation, rather than the rebound itself activating DCs and T cells¹⁰³, and that the homeostatic proliferation reconstituted the pre-TMZ T cell repertoire metrics and not selectively expanding T cell clones¹⁰⁵ as observed with the addition of TTFields (FIGS. 6A-6H; FIGS. 31A-31B).

In fact, the sustained immunosuppressive effects of TMZ at the standard dosing, including lymphopenia, an exhausted T cell state, and increased MDSCs and T_{regs}, are commonly observed in GBM and other tumors^{42, 104, 106-112} and largely opposite to the selective expansion or activation or both of pDCs, cDCs, T1IFN-targted NK and monocyte subtypes, and TCR clonal expansion observed with TTFields treatment in humans (TTFields+TMZ) and the KR158 model (TTFields alone). Since TTFields plus TMZ is an established treatment standard at our institution and many others, future studies should focus on comparing the immune status of adjuvant TTFields plus TMZ to TTFields alone, especially in MGMT-unmethylated GBM that are relatively resistant to TMZ¹ but not to TTFields⁵. In addition, our data provide a compelling rationale for combining TTFields with immune checkpoint inhibitors to create a potential therapeutic synergy. A gene signature for TTFields' immunological effects has been identified in this study (FIG. 6F, Table 7).

### Example 6—Materials and Methods

Antibodies

For immunofluorescence and Western blotting, the following was used: Primary antibodies—LAMIN A/C (Santa Cruz, Cat #sc-7292 and 376248-AF488), cGAS (Santa Cruz, Cat #sc-515802), STING (Novus Biologicals, Cat #NBP2-2468355), AIM2 (CST, Cat #12948; Proteintech, Cat #14357-1-AP), IRF3 (Santa Cruz, Cat #sc-33641), p-IRF3 (CST, 29047S), p65 (Santa Cruz, Cat #sc-8008), p-p65 (Santa Cruz, Cat #sc-136548), GSDMD (SIGMA, Cat #G7422), caspase-1 (Santa Cruz, Cat #sc-514), ActinGreen 488 (Thermo Fisher, Cat #R37110), β-tubulin (Santa Cruz, Cat #sc-5274), and β-actin (Santa Cruz, Cat #sc-47778). Secondary antibodies—goat anti-mouse-A555 (Jackson Immunoresearch, Cat #111-295-003), goat anti-rabbit IgG-A647 (Jackson Immunoresearch, Cat #111-605-003), HRP-conjugated anti-mouse (Santa Cruz, Cat #sc-516102), and HRP-conjugated anti-rabbit (Enzo, Cat #ADI-SAB-300-J). For FACS: Antibodies were purchased from Biolegend and diluted at 1:200 unless otherwise specified: CD45 (clone: 30-F11, Cat #103126, 103108, 103112), MHC II (clone: M5/114.15.2, Invitrogen, Cat #48-5321-32, 1:400), CD4 (clone: RM4-5, Invitrogen, Cat #47-0042-80), CD44 (clone: IM7, Cat #103012, 1:100), ly6g/ly6c (clone: RB6-8C5, Cat #108411), CD8α (clone: 53-6.7, Cat #100721), CD11b (clone: M1/70, Cat #101215), CD80 (clone: 16-10A1, Cat #104733), CD62L (clone: MEL-14, Cat #104405), CD86 (clone: GL-1, Cat #105005, 1:150), CD69 (clone: H1.2F3, Cat #104507), F4/80 (clone: BM8, Cat #123110), and CD11c (clone: N418, Cat #117307, 1:100).

Cell Culture

The following was used for cell culture: HEK 293T (from ATCC) and human GBM cells U87MG (from ATTC), LN428¹, LN827², A549, and PANC-1 (from ATTC) were grown DMEM media supplemented with 10% FBS and 1% pen/strep and the mouse GBM cell line KR158-luc³ in RIPA 1640 media supplemented with 10% FBS and 1% pen/strep. To produce lentivirus, PEI (1 μg/μl) was used at a 2:1 ratio of PEI (μg):total DNA in the pLKO.1 backbone (μg) to transfect HEK 293T cells. PSPAX2 and PMD2.G plasmids were used for viral packaging and enveloping, respectively in advanced DMEM media supplemented with 1.25% FBS, 10 mM HEPES, 1X Pyruvate and 10 mM Sodium butyrate. TTFields were applied to cancer cell lines using the Inovitro™ system (Novocure, Israel). Cells were treated with TTFields at frequencies of 200 kHz (U87, LN827, LN428 and KR158-luc) and 150 kHz (A549, PANC-1).

Immunofluorescence

Cells grown on cover slips were fixed with 4% paraformaldehyde for 30 min at 4° C., incubated overnight at 4° C. with different combinations of primary antibodies (dilution 1:500) against indicated antigens and then for 2 hours at room temperature with appropriate fluorochrome-conjugated secondary antibodies, (dilution 1:500). Labeled cells were counterstained with DAPI (Thermo Fisher, Cat #D1306) at 1 μgm/ml and images captured and analyzed, using a Zeiss 800 inverted confocal microscope. Images were captured at 63X oil immersion objective, keeping all the conditions of microscope, exposure and software settings identical for all samples.

Quantitative RT-PCR

QIAGEN RNeasy Mini Kit (Cat #74106) was used to extract RNA from cells/tissues according to the manufacturer's protocol. One μg total RNA was subjected to reverse transcription using iScript cDNA Synthesis Kit (BIO-RAD, Cat #1708891). qPCR was performed using PowerUp SYBR Green Master Mix (Applied Biosystems, Cat #A25741) and on QuantStudio 3 from Applied Biosystems. Primers used are as follow:

| | hISG15 |
|---|---|
| | forward (fw) |
| (SEQ ID NO: 1) | |
| | GGTGGACAAATGCGACGAA, |
| | reverse (rev) |
| (SEQ ID NO: 2) | |
| | TGCTGCGGCCCTTGTTAT; |
| | hCXCL10 |
| | fw |
| (SEQ ID NO: 3) | |
| | AAGTGCTGCCGTCATTTTCT, |
| | rev |
| (SEQ ID NO: 4) | |
| | CCTATGGCCCTCATTCTCAC; |
| | hSTING |
| | fw |
| (SEQ ID NO: 5) | |
| | GCCAGCGGCTGTATATTCTC, |
| | rev |
| (SEQ ID NO: 6) | |
| | GCTGTAAACCCGATCCTTGA; |
| | hIFNα |
| | fw |
| (SEQ ID NO: 7) | |
| | GACTCCATCTTGGCTGTGA, |
| | rev |
| (SEQ ID NO: 8) | |
| | TGATTTCTGCTCTGACAACCT; |
| | hIFNβ |
| | fw |
| (SEQ ID NO: 9) | |
| | GAATGGGAGGCTTGAATACTGCCT, |
| | rev |
| (SEQ ID NO: 10) | |
| | TAGCAAAGATGTTCTGGAGCATCTC; |
| | hGAPDH |
| | fw |
| (SEQ ID NO: 11) | |
| | GGCATGGACTGTGGTCATGA, |
| | rev |
| (SEQ ID NO: 12) | |
| | ACCACCATGGAGAAGGC; |
| | hIL1α |
| | fw |
| (SEQ ID NO: 13) | |
| | TGTAAGCTATGGCCCACTCCA, |
| | rev |
| (SEQ ID NO: 14) | |
| | AGAGACACAGATTGATCCATGCA; |
| | hIL1β |
| | fw |
| (SEQ ID NO: 15) | |
| | CTCTCTCCTTTCAGGGCCAA, |
| | rev |
| (SEQ ID NO: 16) | |
| | GAGAGGCCTGGCTCAACAAA; |
| | hIL6 |
| | fw |
| (SEQ ID NO: 17) | |
| | CACCGGGAACGAAAGAGAAG, |
| | rev |
| (SEQ ID NO: 18) | |
| | TCATAGCTGGGCTCCTGGAG; |
| | hIL8 |
| | fw |
| (SEQ ID NO: 19) | |
| | ACATGACTTCCAAGCTGGCC, |
| | rev |
| (SEQ ID NO: 20) | |
| | CAGAAATCAGGAAGGCTGCC; |
| | hAIM2 |
| | fw |
| (SEQ ID NO: 21) | |
| | GCTGCACCAAAAGTCTCTCC, |
| | rev |
| (SEQ ID NO: 22) | |
| | ACATCTCCTGCTTGCCTTCT; |
| | hIFIT1 |
| | fw |
| (SEQ ID NO: 23) | |
| | TGAAGTGGACCCTGAAAACC; |
| | hIFIT1 |
| | rev |
| (SEQ ID NO: 24) | |
| | TAAAGCCATCCAGGCGATAG; |
| | hMX1 |
| | fw |
| (SEQ ID NO: 25) | |
| | GGGAAGGAATGGGAATCAGT; |
| | hMX1 |
| | rev |
| (SEQ ID NO: 26) | |
| | CCCACAGCCACTCTGGTTAT; |
| | hIFI44L |
| | fw |
| (SEQ ID NO: 27) | |
| | GATGAGCAACTGGTGTGTCG; |
| | hIFI44L |
| | rev |
| (SEQ ID NO: 28) | |
| | ACTGACGGTGGCCATAAAAC; |
| | mAIM2 |
| | fw |
| (SEQ ID NO: 29) | |
| | CATGGAGGTCACCAGTTCCT, |
| | rev |
| (SEQ ID NO: 30) | |
| | TTTGTTTTGCTTGGGTTTCC; |
| | mIFNβ |
| | fw |
| (SEQ ID NO: 31) | |
| | CCCTATGGAGATGACGGAGA, |
| | rev |
| (SEQ ID NO: 32) | |
| | CTGTCTGCTGGTGGAGTTCA; |
| | mIL6 |
| | fw |
| (SEQ ID NO: 33) | |
| | CCGGAGAGGAGACTTCACAG, |
| | rev |
| (SEQ ID NO: 34) | |
| | TCCACGATTTCCCAGAGAAC; |
| | mISG15 |
| | fw |
| (SEQ ID NO: 35) | |
| | AAGAAGCAGATTGCCCAGAA, |
| | rev |
| (SEQ ID NO: 36) | |
| | CGCTGCAGTTCTGTACCAC; |
| | mIFIT1 |
| | fw |
| (SEQ ID NO: 37) | |
| | GCCCAGATCTACCTGGACAA; |
| | mIFIT1 |
| | rev |
| (SEQ ID NO: 38) | |
| | CCTCACAGTCCATCTCAGCA; |
| | mMX1 |
| | fw |
| (SEQ ID NO: 39) | |
| | TGTGCAGGCACTATGAGGAG; |
| | mMX1 |
| | rev |
| (SEQ ID NO: 40) | |
| | ACTCTGGTCCCCAATGACAG; |
| | mIFI44L |
| | fw |
| (SEQ ID NO: 41) | |
| | GGGGTCTGACGAAAGCAGTA; |
| | mIFI44L |
| | rev |
| (SEQ ID NO: 42) | |
| | CCCATTGAATCACACAGCAT; |
| | mSTING |
| | fw |
| (SEQ ID NO: 43) | |
| | GTTTGCCATGTCACAGGATG, |
| | rev |
| (SEQ ID NO: 44) | |
| | CAATGAGGCGGCAGTTATTT; |
| | mGAPDH |
| | fw |
| (SEQ ID NO: 45) | |
| | GGAGCGAGACCCCACTAACA, |
| | rev |
| (SEQ ID NO: 46) | |
| | ACATACTCAGCACCGGCCTC. |

Western Blotting

Cells were treated on ice for 20 min with RIPA buffer (150 mM NaCl, 1% NP-40, 0.5% Sodium deoxycholate, 0.1% SDS, 25 mM, PH 7.4 Tris) containing a protease inhibitor cocktail (Roche), followed by centrifugation at 13,000g at 4° C. for 20 min. Supernatants were collected and protein concentration determined using a protein assay dye reagent (Bio-Rad). Equal amounts of proteins were resolved by SDS-PAGE and transferred onto polyvinylidene difluoride (PVDF) membranes. Membranes were blocked with 5% non-fat milk in TBST, then probed with indicated primary antibodies (1:500) at 4° C. overnight, washed with TBST, and incubated with HRP-conjugated anti-rabbit or anti-mouse secondary antibodies (1:500) at room temperature for 1 hour.

Flow Cytometry

Single cell suspensions were Fc-blocked cells before incubation with the indicated fluorochrome-conjugated antibodies for 20 min at 4° C. in the dark. FACS were performed on a BD FACS Canto II and analyzed by FlowJo_V10. Live cells were separated from debris in an SSC-A (y) versus FSC-A (x) dot plot, doublets excluded with FSC-H (y) versus FSC-A (x)/SSC-H (y) versus SSC-A (x) dot plots. Singlets were analyzed and gated as indicated.

Caspase-1 Activation Assay

Caspase-1 activation assay was performed according to the manufacturer's protocol (FAM-FLICA® Caspase-1 Assay Kit, ImmunoChemistry, Cat #97). Adherent cells were trypsinized and washed twice in wash buffer, resuspended and incubated with FLICA at the dilution of 1:30 at 37° C. for 1 hour, washed and analyzed by BD FACS Canto II at the channel of FTIC. Debris and doublets were excluded out from analysis.

ELISA

Cell culture media or total cell lysates were analyzed using the DuoSet® ELISA Development Systems (R&D, Cat #DY007). Plates were coated with a primary antibody (R&D, Cat #DY814-05) overnight one day ahead of the assay. Samples and standards were added to primary antibody coated plates in duplicate, incubated for 2 hours at room temperature each with biotinylated antibody and HRP-conjugated streptavidin, followed by 20 min incubation with HRP color reagent, with 3 washes between each step. After the stop solution was added, optical density at 450 nm-570 nm was measure. Sample quantification was calculated according to the standard curve.

LDH Release Assay

Cell culture media were incubated for 30 minutes at room temperature with equal volume of the CytoTox 96 Non-Radioactive Cytotoxicity Assay reagent according to the manufacturer's protocol (Promega, Cat #G1780). Absorbance at 490 nm wavelength was measured using a Molecular Device SpectraMax i3x microplate reader. The data was presented as LDH release (%)=[(unknown-negative)/(positive-negative)]×100%.

Co-Culture Experiment

Two sets of 2×10⁴ KR158-luc cells stably expressing a scrambled shRNA or shRNA against STING or AIM2 or both were seeded in each 60 mm ceramic dish, then left untreated or treated with TTFields at 200 kHz for 3 days. Supernatants were then collected, filtered using 0.45 μm filters, and then added to a 12-well plate with each well containing RPMI with 10% FBS and 10⁶ splenocytes freshly harvested from 6-8 weeks old male C57BL/6J mice, and cocultured for 3 days. On days 4 and 5, culture media from the remaining ceramic dishes were collected to replenish the co-culture. On day 6, co-cultured splenocytes (CD45±) were immunophenotyped by flow cytometry.

Intracranial Vaccination Protocol

All animal experiments were performed according to regulations and rule of institutional IACUC. KR158-luc cells stably expressing a scrambled shRNA or shRNA against STING, AIM2 or both were untreated or treated with TTFields at 200 kHz for 3 days. 3×10⁵ of these TTFields-treated KR158-luc cells suspended in 3 μl PBS were implanted slowly (1 μl/min) in the posterior frontal lobe of the brain with 6-week-old male syngeneic C57BL/6J mice (Jackson Laboratory), at 2 mm lateral to the right and 3.5 mm deep with bregma as the reference point using an automated mouse stereotaxic apparatus (Stoelting's). Orthotopic tumor growth was monitored by bioluminescence imaging (see below). One cohort was euthanized at 2 weeks after implantation for immunophenotyping, while the rest were allowed to proceed to the survival endpoint. For immunophenotyping, blood, cervical lymph nodes, spleen, bone marrow were collected and digested to single cell suspension, filtered through 40 μm filters and subjected to red blood cell lysis using a lysis buffer (BD, Cat #555899), if necessary. Mouse brains were embedded in OCT and stored at −80° C. until analysis.

For the rechallenge experiment, 6×10⁵ parental KR158-luc cells in 5 μl PBS were injected intracranially into surviving mice at day 100 post initial injection and age- and sex-matched naïve mice. At 1 and 2 weeks after re-challenge, PBMCs were collected through tail-vein phlebotomy for immunophenotyping. At 20 weeks post re-challenge, surviving mice were euthanized and dcLNs, blood and spleens were collected for immunophenotyping. For control, a cohort of age- and sex-matched naïve mice were implanted orthotopically with 6×10⁵ parental KR158-luc cells in 5 μl PBS and the same tissues collected 2 weeks later for the same immunophenotyping analysis.

In Vivo Imaging System (IVIS) Spectrum

To monitor brain tumor growth, animals were imaged using the IVIS system (Xenogen). Mice were anesthetized by isoflurane (5% induction and 2% maintenance). RediJect D-Luciferin Bioluminescent Substrate (PerkinElmer, Cat #ULO8RV01) were injected into mice subcutaneously and images taken repeatedly until the bioluminescence signal reached its peak. The data was analyzed using Living Image software (Caliper Life Sciences).

Single Cell PBMC RNA-Seq Analysis

Sample Processing

Cryopreserved PBMCs from patients were washed with PBS and viability verified by Trypan Blue staining (Supplementary Table S2). Single cell suspensions were loaded onto Chromium Single Cell Chip (10× Genomics) according to the manufacturer's instructions at a target capture rate of approximately 10,000 cells/sample. The pooled single-cell RNA-seq libraries were prepared using the Chromium Single Cell 3′ Solution (10× Genomics) according to the manufacturer's instructions. All paired samples of pre-TTFields (pre-TTF) and post-TTFields (post-TTF) treatment for each patient and the resulting libraries were processed in parallel in the same batch. In total, there were 3 batches. All single cell libraries were sequenced with an 8-base i7 sample index read, including a 28-base read 1 containing cell barcodes and unique molecular identifiers (UMI) and a 150-base read 2 for mRNA insert on Illumina Novaseq. Sample characteristics are summarized in Supplementary Table S3.

Data Processing

The main operations were performed using the Seurat R package (3.2.2)^{4, 5}, unless otherwise stated. When option parameters for function deviated from the default values, details of the changes were provided. Most of the changes to the default options were made to accommodate and leverage the large size of the dataset.

Cell Ranger Aggregation: Conversion of the raw sequencing data from the bcl to fastq format and the subsequent alignment to the reference genome GRCh38 (GENCODE v.24) and gene count were performed using the cellranger software (10× Genomics, version 4.0.0) with the command cellranger mkfastq, the STAR aligner, and the command cellranger count, respectively. Results from all libraries and batches were pooled together using the command cellranger aggr without normalization for dead cells as it will be handled downstream. The filtered background feature barcode matrix obtained from this step was used as input for sequential analysis.

Normalization of UMI: Using the global scaling normalization method, the feature expression for each cell was divided by the total expression, multiplied by the scale factor (10,000), and log transformed using the Seurat R function NormalizeData with method “Log Normalize”.

Seurat aggregation and correction for batch effect: As the counts were from three different batches, to align cells and eliminate batch effects for dimension reduction and clustering, a multi dataset integration strategy was adopted as previously described⁵. Briefly, “anchors cells” were identified between pairs of datasets and used to normalize multiple datasets from different batches. Given the size of our datasets (a total of 193760 cells), a reference-based, reciprocal PCA variant of the method detailed in the Seurat R package was chosen^{4, 5}. First, the previously integrated dataset was split by batches, using the Seurat function SplitObject. Next, for each split object, variable feature selection was performed using the function FindVariableFeatures. Features for integration were selected using the function SelectIntegrationFeatures and PCA performed for each split object on the selected features. The anchor cells were identified by using the function FindIntegrationAnchors with the reference chosen as the largest among 3 batches and the reduction option set to ‘rpca’. Finally, the whole datasets from 3 batches were reintegrated using the function IntegrateData with the identified anchor cells.

UMAP dimension reduction: The integrated multiple batch dataset was used as input for UMAP dimension reduction⁶. The feature expression was scaled using the Seurat function ScaleData, followed by a PCA run using the function RunPCA (Seurat) with the total number of principal components (PC) to compute and store option of 100. The UMAP coordinates for single cells were obtained using the RunUMAP function (Seurat) with the top 75 PCs as input features (dims=1:75) with min.dist=0.75 and the number of training epochs n.epochs=2000.

Clustering of cells: A graph-based clustering approach was implemented in the Seurat package, which embeds cells in a K-nearest neighbor graph with edges drawn between similar cells and partitions nodes in the network into communities. Briefly, a Shared Nearest Neighbor graph was constructed using the FindNeigbhors function with an option dimension of reduction input dims=1:75, error bound nn.eps=0.5. This function calculates the neighborhood overlap (Jaccard index) between every cell and its k.param nearest neighbors⁷. The graph was partitioned into clusters using the FindClusters function with different values for resolution parameter. The higher the resolution, the smaller the cluster size. Resolutions values of 0.1, 1, 3, 5, 10 were tested. Resolution 0.3 gave large clusters of all major cell types such as B and T cells without cell subtypes. Resolutions 3, 5 and 10 gave excessively small clusters, which are mostly patient specific making cross-patients generalization difficult. Resolutions 1 was chosen to perform downstream analyses as it produced reasonable cluster sizes, partitioning cells into biologically recognized cell subtypes. The differential expressed gene markers for each cluster were found using the FindAllMarkers function with the option of only returning positive markers and a minimal fraction of cells with the marker of 0.25. The default Wilcoxon Rank Sum test was used to calculate statistical differences in each cell cluster.

Analytical plan in dead cell exclusion: In all above analyses, dead cells were not filtered out before clustering, rather cluster-based dead cell exclusion was used. Filtering out dead cells was tested before clustering by mitochondrial genes content and the average number of read UMI or average number of UMI per gene. A reasonable threshold for the particular patient dataset was not identified. Even using a relaxed threshold for mitochondrial content <15% eliminated more than 40% of cells in some patients. Even though Trypan Blue staining was used to estimate the dead cell fraction, the dead cell fraction was never more than 10% (Table S2). The abnormal elevated content of mitochondrial genes in our dataset may be due to the significant stresses that these patients were under, including cancer diagnosis, recent radiotherapy and chemotherapy, and TTFields treatment, and steroid treatment, etc. As a result, all cells were analyzed without prefiltration for dead cells before clustering. Instead, dead cells were identified after clustering and dead cells formed a smear cluster (Clusters 16, 24, 28, and 30 in Resolution 1 UMAP) in the center of the UMAP map without clear cell-specific identity and with elevated mitochondrial genes and housekeeping genes. Cells in these clusters were excluded from further analysis.

TTFields Treatment Analysis

Cluster proportion changes. The cluster proportion change after TTFields treatment for each cluster was performed using Wilcoxon signed rank test on paired values of log proportion of each patient pre TTFields and post TTFields treatment using wilcox.test with option pair=TRUE in R programming environment (version 4.0.3).

Correlation between cluster proportion changes and diversity changes. The log fold changes for each patient's cluster proportions and TCR diversity indices between pre TTFields and post TTFields treatment were calculated. Next, the Spearman correlation test was performed using the log FC of proportion changes and TCR diversity changes as input using the function cor.test, method=“spearman”, R programming environment (version 4.0.3).

Gene differential expression analysis between pre TTFields and post TTFields paired samples. The differential expression analysis was done using the LIMMA/Voom method (LIMMA R package)⁸⁻¹°. Briefly, the single cell UMI counts matrix for each cluster was transformed to log 2-counts per million (log CPM) with an estimate for the mean-variance relationship and used to compute appropriate observation-level weights using the voom function. The transformed matrix was then fitted to a linear model with timepoints and patients as factors using the function in/fit. A moderated t-statistics was computed using empirical Bayes moderation of the standard errors towards a global value using the function eBayes. Next, estimated coefficients and standard errors for the contrast of pre TTFields and post TTFields timepoints was calculated using the function contrasts.fit. Contrast-specific, moderated t-statistics was then computed using the function eBayes. The log FC, t-statistics, p values were exported using the function top Table.

Pathway differential expression analysis. A Gene Set Enrichment Analysis (GSEA) was used as previously described¹¹ for analysis of immune specific pathways of interest. For each cluster, all genes were ranked using the moderated t-test from the Gene differential expression analysis step above. Then GSEA (preranked, “classic” mode, 10,000 permutations) was performed to calculate enrichment for the pathways of interest, using the command lines and Java implementation of GSEA downloaded from http://software.broadinstitute.org/gsea/index.jsp.

Heatmaps of log FC of gene expression and pathway activity for each cluster. For each cluster, the gene counts for each library were calculated by summing up all the associated UMI counts of cells and normalized to transcript per million (tpm) unit by dividing the counts by the length of the genes in kilobases to obtain read per kilobase (RPK). RPK was normalized by dividing to the total RPK values of each library and expressed in millions, and log 2 transformed. The log FC of gene expression of each patient between pre TTFields and post TTFields treatment was then calculated by subtracting the respective log tpm values.

For the global pathway activity log FC calculation, pathways and gene membership were downloaded from Gene Ontology http://geneontology.org/, selecting only those related to Biological Process. The activity for each pathway was calculated as an average tpm value of all genes in that pathway, and the log FC of a pathway of each patient between pre TTFields and post TTFields treatment calculated by dividing the pathway activity of post TTFields values by the pre TTFields values, followed by report and visualization by heatmaps.

Heatmaps of TING pathway scores at the single cell level. The score was defined as the mean expression (normalized by Seurat function NormalizeData; in brief, feature counts for each cell are divided by the total counts for that cell and multiplied by 10000 and then natural-log transformed using log 1p) of genes annotated as belonging to the Gene Ontology “response to type I interferon” GO:0034340. The gene set was downloaded from //www.gseamsigdb.org, and included 99 genes: ABCE1, ADAR, BST2, CACTIN, CDCl37, CNOT7, DCST1, EGR1, FADD, GBP2, HLA-A, HLA-B, HLA-C, HLA-E, HLA-F, HLA-G, HLA-H, HSP90AB1, IFI27, IFI35, IFI6, IFIT1, IFIT2, IFIT3, IFITM1, IFITM2, IFITM3, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IKBKE, IP6K2, IRAK1, IRF1, IRF2, IRF3, IRF4, IRF5, IRF6, IRF7, IRF8, IRF9, ISG15, ISG20, JAK1, LSM14A, MAVS, METTL3, MIR21, MMP12, MUL1, MX1, MX2, MYD88, NLRC5, OAS1, OAS2, OAS3, OASL, PSMB8, PTPN1, PTPN11, PTPN2, PTPN6, RNASEL, RSAD2, SAMHD1, SETD2, SHFL, SHMT2, SP100, STAT1, STAT2, TBK1, TREX1, TRIM56, TRIM6, TTLL12, TYK2, UBE2K, USP18, WNT5A, XAF1, YTHDF2, YTHDF3, ZBP1.

Bulk RNA-Seq of Isolated T Lymphocytes and TCR Clonotyping

Sample Preparation

Untouched T cells were selected from PBMC single cell suspension using human pan T Cell isolation kit according to the manufacturer's instructions (Miltenyi Biotec, Cat #130-096-535). RNA was extracted utilizing QIAGEN RNeasy Midi Kit (Cat #75144) according to the manufacturer's instructions. Bulk RNAseq library was constructed, pooled and sequenced on a NovaSeq 6000 Illumina instrument at University of Florida Interdisciplinary Center for Biotechnology Research Gene Expression & Genotyping/NextGen Sequencing Core.

Sequencing Analysis

Paired-end reads were trimmed with trimmomatic v/0.36. Alignment and gene counts were generated against the GRCh38.p12 genome assembly using the annotation GeneCode release 28 by STAR v2.6.0b with default options and quantmode=GeneCounts (Table S4). The heatmaps of log FC of gene expression and pathway activity were made similarly to the described procedure in sing cell analysis above.

TCR Clonotyping

To extract the T Cell receptor clones from bulk RNA-seq data, the pair end reads from bulk non-targeted RNA-seq were supplied to MiXCR v.3.0.13, an universal tool for analyzing T- and B-cell receptor repertoire sequencing data (https://milaboratory.com/software/mixcr/), using the command analyze shotgun with the option of starting-material ma, only-productive. This command performed complicated pipeline, including alignment of raw sequencing reads, assembly of overlapping fragmented reads, inputting good TCR alignments, assembly of aligned sequences into clonotypes and exporting the resulting clonotypes into a tab-delimited file. For each sample, the Inverse Simpson Index was calculated using the vdjtools v 1.2.1 (https://github.com/mikessh/vdjtools) with the command CalcDiversityStats and input of clonotypes from the previous MixCR step. The clonal change plot was created using the Immunoarch R package v0.6.5 (https://cloud.rproject.org/web/packages/immunarch/index.html) with the function trackClonotypes, option col=“a.a”, to collapse all clones that share the same amino acid sequences.

Statistical Analyses

GraphPad Prism 8 software was used for statistical analysis. All statistical tests were two-sided and P values ≤0.05 (with 95% confidence interval) considered statistically significant for each of the specific statistical comparisons (*P<0.05, **P<0.01, ***P<0.001, ****P<0.0001). Data with continuous outcomes are represented as mean±s.e.m. For scRNA-seq, the comparison was based on annotated clusters comparing before and after treatment for each patient.

The methods described herein can also be applied in the in vivo context by applying the alternating electric fields to a target region of a live subject's body (e.g., using the Novocure Optune® system). This may be accomplished, for example, by positioning electrodes on or below the subject's skin so that application of an AC voltage between selected subsets of those electrodes will impose the alternating electric fields in the target region of the subject's body.

For example, in situations where the relevant cells are located in the subject's brain, one pair of electrodes could be positioned on the front and back of the subject's head, and a second pair of electrodes could be positioned on the right and left sides of the subject's head. In some embodiments, the electrodes are capacitively coupled to the subject's body (e.g., by using electrodes that include a conductive plate and also have a dielectric layer disposed between the conductive plate and the subject's body). But in alternative embodiments, the dielectric layer may be omitted, in which case the conductive plates would make direct contact with the subject's body. In another embodiment, electrodes could be inserted subcutaneously below a patient's skin. An AC voltage generator applies an AC voltage at a selected frequency (e.g., 200 kHz) between the right and left electrodes for a first period of time (e.g., 1 second), which induces alternating electric fields where the most significant components of the field lines are parallel to the transverse axis of the subject's body.

Then, the AC voltage generator applies an AC voltage at the same frequency (or a different frequency) between the front and back electrodes for a second period of time (e.g., 1 second), which induces alternating electric fields where the most significant components of the field lines are parallel to the sagittal axis of the subject's body. This two step sequence is then repeated for the duration of the treatment. Optionally, thermal sensors may be included at the electrodes, and the AC voltage generator can be configured to decrease the amplitude of the AC voltages that are applied to the electrodes if the sensed temperature at the electrodes gets too high. In some embodiments, one or more additional pairs of electrodes may be added and included in the sequence. In alternative embodiments, only a single pair of electrodes is used, in which case the direction of the field lines is not switched. Note that any of the parameters for this in vivo embodiment (e.g., frequency, field strength, duration, direction-switching rate, and the placement of the electrodes) may be varied as described above in connection with the in vitro embodiments. But care must be taken in the in vivo context to ensure that the electric field remains safe for the subject at all times.

Note that in the experiments described herein, the alternating electric fields were applied for an uninterrupted interval of time (e.g., 72 hours or 14 days). But in alternative embodiments, the application of alternating electric fields may be interrupted by breaks that are preferably short. For example, a 72 hour interval of time could be satisfied by applying the alternating electric fields for six 12 hour blocks, with 2 hour breaks between each of those blocks.

### REFERENCES

1. Stupp, R. et al. Effects of radiotherapy with concomitant and adjuvant temozolomide versus radiotherapy alone on survival in glioblastoma in a randomised phase III study: 5-year analysis of the EORTC-NCIC trial. Lancet Oncol 10, 459-466, doi:10.1016/S1470-2045(09)70025-7 (2009).
2. Lai, A. et al. Phase II study of bevacizumab plus temozolomide during and after radiation therapy for patients with newly diagnosed glioblastoma multiforme. J Clin Oncol 29, 142-148, doi:10.1200/JCO.2010.30.2729 (2011).
3. Nduom, E. K., Weller, M. & Heimberger, A B Immunosuppressive mechanisms in glioblastoma. Neuro-Oncology 17, vii9-vii14, doi:10.1093/neuonc/nov151 (2015).
4. Sampson, J. H., Gunn, M. D., Fecci, P. E. & Ashley, D. M. Brain immunology and immunotherapy in brain tumours. Nature Reviews Cancer 20, 12-25, doi:10.1038/s41568-019-0224-7 (2020).
5. Stupp, R. et al. Effect of Tumor-Treating Fields Plus Maintenance Temozolomide vs Maintenance Temozolomide Alone on Survival in Patients With Glioblastoma: A Randomized Clinical Trial. JAMA 318, 2306-2316, doi:10.1001/jama.2017.18718 (2017).
6. Ceresoli, G. L. et al. Tumour Treating Fields in combination with pemetrexed and cisplatin or carboplatin as first-line treatment for unresectable malignant pleural mesothelioma (STELLAR): a multicentre, single-arm phase 2 trial. Lancet Oncol 20, 1702-1709, doi:10.1016/S1470-2045(19)30532-7 (2019).
7. Stupp, R. et al. NovoTTF-100A versus physician's choice chemotherapy in recurrent glioblastoma: a randomised phase III trial of a novel treatment modality. Eur J Cancer 48, 2192-2202, doi:10.1016/j.ejca.2012.04.011 (2012).
8. Villano, J. L. et al. Delayed response and survival from NovoTTF-100A in recurrent GBM. Medical Oncology 30, 338, doi:10.1007/s12032-012-0338-1 (2013).
9. Vymazal, J. & Wong, E. T. Response Patterns of Recurrent Glioblastomas Treated With Tumor-Treating Fields. Seminars in Oncology 41, S14-S24, doi:https://doi.org/10.1053/j.seminoncol.2014.09.009 (2014).
10. Hottinger, A. F., Pacheco, P. & Stupp, R. Tumor treating fields: a novel treatment modality and its use in brain tumors. Neuro-Oncology 18, 1338-1349, doi:10.1093/neuonc/now182 (2016).
11. Kirson, E. D. et al. Alternating electric fields (TTFields) inhibit metastatic spread of solid tumors to the lungs. Clin Exp Metastasis 26, 633-640, doi:10.1007/s10585-009-9262-y (2009).
12. Voloshin, T. et al. Tumor-treating fields (TTFields) induce immunogenic cell death resulting in enhanced antitumor efficacy when combined with anti-PD-1 therapy. Cancer Immunology, Immunotherapy, doi:10.1007/s00262-020-02534-7 (2020).
13. Giladi, M. et al. Mitotic Spindle Disruption by Alternating Electric Fields Leads to Improper Chromosome Segregation and Mitotic Catastrophe in Cancer Cells. Sci Rep 5, 18046, doi:10.1038/srep18046 (2015).
14. Kessler, A. F. et al. Effects of tumor treating fields (TTFields) on glioblastoma cells are augmented by mitotic checkpoint inhibition. Cell death discovery 4, 12-12, doi:10.1038/s41420-018-0079-9 (2018).
15. Allen, M., Bjerke, M., Edlund, H., Nelander, S. & Westermark, B. Origin of the U87MG glioma cell line: Good news and bad news. Science Translational Medicine 8, 354re353-354re353, doi:10.1126/scitranslmed.aaf6853 (2016).
16. Van Meir, E. G. et al. Analysis of the p53 gene and its expression in human glioblastoma cells. Cancer Res 54, 649-652 (1994).
17. Ishii, N. et al. Frequent co-alterations of TP53, p16/CDKN2A, p14ARF, PTEN tumor suppressor genes in human glioma cell lines. Brain Pathol 9, 469-479, doi:10.1111/j.1750-3639.1999.tb00536.x (1999).
18. Ishikawa, H., Ma, Z. & Barber, G. N. STING regulates intracellular DNA-mediated, type I interferon-dependent innate immunity. Nature 461, 788-792, doi:10.1038/nature08476 (2009).
19. Barber, G. N. STING: infection, inflammation and cancer. Nat Rev Immunol 15, 760-770, doi:10.1038/nri3921 (2015).
20. Li, X. et al. Cyclic GMP-AMP Synthase Is Activated by Double-Stranded DNA-Induced Oligomerization Immunity 39, 1019-1031, doi:https://doi.org/10.1016/j.immuni.2013.10.019 (2013).
21. Hornung, V. et al. AIM2 recognizes cytosolic dsDNA and forms a caspase-1-activating inflammasome with ASC. Nature 458, 514-518, doi:10.1038/nature07725 (2009).
22. Fernandes-Alnemri, T., Yu, J.-W., Datta, P., Wu, J. & Alnemri, E. S. AIM2 activates the inflammasome and cell death in response to cytoplasmic DNA. Nature 458, 509-513, doi:10.1038/nature07710 (2009).
23. Bürckstümmer, T. et al. An orthogonal proteomic-genomic screen identifies AIM2 as a cytoplasmic DNA sensor for the inflammasome. Nature Immunology 10, 266-272, doi:10.1038/ni.1702 (2009).
24. Paludan, S. R. & Bowie, A. G. Immune sensing of DNA Immunity 38, 870-880, doi:10.1016/j.immuni 2013.05.004 (2013).
25. Motwani, M., Pesiridis, S. & Fitzgerald, K. A. DNA sensing by the cGAS-STING pathway in health and disease. Nature Reviews Genetics 20, 657-674, doi:10.1038/s41576-019-0151-1 (2019).
26. Di Micco, A. et al. AIM2 inflammasome is activated by pharmacological disruption of nuclear envelope integrity. Proc Natl Acad Sci USA 113, E4671-4680, doi:10.1073/pnas.1602419113 (2016).
27. Xia, T., Konno, H., Ahn, J. & Barber, G. N. Deregulation of STING Signaling in Colorectal Carcinoma Constrains DNA Damage Responses and Correlates With Tumorigenesis. Cell Rep 14, 282-297, doi:10.1016/j.celrep.2015.12.029 (2016).
28. Vanpouille-Box, C. et al. DNA exonuclease Trex1 regulates radiotherapy-induced tumour immunogenicity. Nat Commun 8, 15618, doi:10.1038/ncomms15618 (2017).
29. Dittmer, T. A. & Misteli, T. The lamin protein family Genome Biology 12, 222, doi:10.1186/gb-2011-12-5-222 (2011).
30. Schreiber, Katherine H. & Kennedy, Brian K. When Lamins Go Bad: Nuclear Structure and Disease. Cell 152, 1365-1375, doi:https://doi.org/10.1016/j.cell.2013.02.015 (2013).
31. Jin, D., Tran, N., Thomas, N. & Tran, D. D. Combining CDK4/6 inhibitors ribociclib and palbociclib with cytotoxic agents does not enhance cytotoxicity. PLoS One 14, e0223555, doi:10.1371/journal.pone.0223555 (2019).
32. Lieber, M., Todaro, G., Smith, B., Szakal, A. & Nelson-Rees, W. A continuous tumor-cell line from a human lung carcinoma with properties of type II alveolar epithelial cells. International Journal of Cancer 17, 62-70, doi:10.1002/ijc.2910170110 (1976).
33. Lieber, M., Mazzetta, J., Nelson-Rees, W., Kaplan, M. & Todaro, G.

Establishment of a continuous tumor-cell line (PANC-1) from a human carcinoma of the exocrine pancreas. International Journal of Cancer 15, 741-747, doi:10.1002/ijc.2910150505 (1975).
34. Rivera, F. et al. Tumor treating fields in combination with gemcitabine or gemcitabine plus nab-paclitaxel in pancreatic cancer: Results of the PANOVA phase 2 study. Pancreatology 19, 64-72, doi:https://doi.org/10.1016/j.pan.2018.10.004 (2019).
35. Pless, M., Droege, C., von Moos, R., Salzberg, M. & Betticher, D. A phase 1/II trial of Tumor Treating Fields (TTFields) therapy in combination with pemetrexed for advanced non-small cell lung cancer. Lung Cancer 81, 445-450, doi:10.1016/j.lungcan.2013.06.025 (2013).
36. Prabakaran, T. et al. Attenuation of cGAS-STING signaling is mediated by a p62/SQSTM1-dependent autophagy pathway activated by TBK1. The EMBO journal 37, e97858, doi:10.15252/embj.201797858 (2018).
37. Gonugunta, V. K. et al. Trafficking-Mediated STING Degradation Requires Sorting to Acidified Endolysosomes and Can Be Targeted to Enhance Anti-tumor Response. Cell reports 21, 3234-3242, doi:10.1016/j.celrep.2017.11.061 (2017).
38. Liu, X. et al. Inflammasome-activated gasdermin D causes pyroptosis by forming membrane pores. Nature 535, 153-158, doi:10.1038/nature18629 (2016).
39. Berghe, T. V. et al. Necroptosis, necrosis and secondary necrosis converge on similar cellular disintegration features. Cell Death & Differentiation 17, 922-930, doi:10.1038/cdd.2009.184 (2010).
40. Krysko, D. V., D'Herde, K. & Vandenabeele, P. Clearance of apoptotic and necrotic cells and its immunological consequences. Apoptosis 11, 1709-1726, doi:10.1007/s10495-006-9527-8 (2006).
41. Flores, C. T. et al. Lin-CCR2⁺ hematopoietic stem and progenitor cells overcome resistance to PD-1 blockade. Nature Communications 9, 4313, doi:10.1038/s41467-018-06182-5 (2018).
42. Karachi, A. et al. Modulation of temozolomide dose differentially affects T-cell response to immune checkpoint inhibition. Neuro-Oncology 21, 730-741, doi:10.1093/neuonc/noz015 (2019).
43. Wildes, T. J. et al. Cross-talk between T Cells and Hematopoietic Stem Cells during Adoptive Cellular Therapy for Malignant Glioma. Clinical Cancer Research 24, 3955-3966, doi:10.1158/1078-0432.Ccr-17-3061 (2018).
44. Ziegler, S. F., Ramsdell, F. & Alderson, M. R. The activation antigen CD69. STEM CELLS 12, 456-465, doi:10.1002/stem.5530120502 (1994).
45. Radulovic, K. et al. The Early Activation Marker CD69 Regulates the Expression of Chemokines and CD4 T Cell Accumulation in Intestine. PLOS ONE 8, e65413, doi:10.1371/journal.pone.0065413 (2013).
46. van Faassen, H. et al. Reducing the stimulation of CD8+ T cells during infection with intracellular bacteria promotes differentiation primarily into a central (CD62LhighCD44high) subset J Immunol 174, 5341-5350, doi:10.4049/jimmunol.174.9.5341 (2005).
47. Hu, J. & August, A. Naïve and Innate Memory Phenotype CD4<sup>+</sup> T Cells Have Different Requirements for Active Itk for Their Development. The Journal of Immunology 180, 6544-6552, doi:10.4049/jimmunol.180.10.6544 (2008).
48. Ma, Q. et al. Lymphatic outflow of cerebrospinal fluid is reduced in glioma. Scientific Reports 9, 14815, doi:10.1038/s41598-019-51373-9 (2019).
49. Knopf, P. M., Cserr, H. F., Nolan, S. C., Wu, T. Y. & Harling-Berg, C. J. Physiology and immunology of lymphatic drainage of interstitial and cerebrospinal fluid from the brain. Neuropathology and Applied Neurobiology 21, 175-180, doi:10.1111/j.1365-2990.1995.tb01047.x (1995).
50. Cserr, H. F. & Knopf, P. M. Cervical lymphatics, the blood-brain barrier and the immunoreactivity of the brain: a new view Immunology Today 13, 507-512, doi:https://doi.org/10.1016/0167-5699(92)90027-5 (1992).
51. Sallusto, F., Geginat, J. & Lanzavecchia, A. Central memory and effector memory T cell subsets: function, generation, and maintenance. Annu Rev Immunol 22, 745-763, doi:10.1146/annurev.immunol.22.012703.104702 (2004).
52. Baaten, B. J. G. et al. CD44 Regulates Survival and Memory Development in Th1 Cells. Immunity 32, 104-115, doi:10.1016/j.immuni.2009.10.011 (2010).
53. Appay, V., van Lier, R. A., Sallusto, F. & Roederer, M. Phenotype and function of human T lymphocyte subsets: consensus and issues. Cytometry A 73, 975-983, doi:10.1002/cyto.a.20643 (2008).
54. Crauste, F. et al. Identification of Nascent Memory CD8 T Cells and Modeling of Their Ontogeny. Cell Syst 4, 306-317 e304, doi:10.1016/j.cels.2017.01.014 (2017).
55. Becht, E. et al. Dimensionality reduction for visualizing single-cell data using UMAP. Nature Biotechnology 37, 38-44, doi:10.1038/nbt.4314 (2019).
56. Guo, X. et al. Global characterization of T cells in non-small-cell lung cancer by single-cell sequencing. Nature Medicine 24, 978-985, doi:10.1038/s41591-018-0045-3 (2018).
57. Li, H. et al. Dysfunctional CD8 T Cells Form a Proliferative, Dynamically Regulated Compartment within Human Melanoma. Cell 176, 775-789.e718, doi:https://doi.org/10.1016/j.cell.2018.11.043 (2019).
58. Greenough, T. C. et al. A Gene Expression Signature That Correlates with CD8<sup>+</sup> T Cell Expansion in Acute EBV Infection. The Journal of Immunology 195, 4185-4197, doi:10.4049/jimmunol.1401513 (2015).
59. Szabo, P. A. et al. Single-cell transcriptomics of human T cells reveals tissue and activation signatures in health and disease. Nature Communications 10, 4706, doi:10.1038/s41467-019-12464-3 (2019).
60. Bratke, K., Kuepper, M., Bade, B., Virchow, J. C., Jr. & Luttmann, W.

Differential expression of human granzymes A, B, and K in natural killer cells and during CD8+ T cell differentiation in peripheral blood. Eur J Immunol 35, 2608-2616, doi:10.1002/eji.200526122 (2005).
61. Vieira Braga, F. A. et al. Blimp-1 homolog Hobit identifies effector-type lymphocytes in humans. Eur J Immunol 45, 2945-2958, doi:10.1002/eji.201545650 (2015).
62. Mackay, L. K. et al. Hobit and Blimp1 instruct a universal transcriptional program of tissue residency in lymphocytes. Science 352, 459-463, doi:10.1126/science.aad2035 (2016).
63. Miragaia, R. J. et al. Single-Cell Transcriptomics of Regulatory T Cells Reveals Trajectories of Tissue Adaptation. Immunity 50, 493-504.e497, doi:10.1016/j.immuni.2019.01.001 (2019).
64. Marzo, A. L., Yagita, H. & Lefrancois, L. Cutting Edge: Migration to Nonlymphoid Tissues Results in Functional Conversion of Central to Effector Memory CD8 T Cells. The Journal of Immunology 179, 36-40, doi:10.4049/jimmunol.179.1.36 (2007).
65. Castellino, F. et al. Chemokines enhance immunity by guiding naïve CD8+ T cells to sites of CD4+ T cell-dendritic cell interaction. Nature 440, 890-895, doi:10.1038/nature04651 (2006).
66. Sallusto, F., Lenig, D., Förster, R., Lipp, M. & Lanzavecchia, A. Two subsets of memory T lymphocytes with distinct homing potentials and effector functions. Nature 401, 708-712, doi:10.1038/44385 (1999).
67. Akondy, R. S. et al. Origin and differentiation of human memory CD8 T cells after vaccination. Nature 552, 362-367, doi:10.1038/nature24633 (2017).
68. Kadowaki, N., Antonenko, S., Lau, J. Y.-N. & Liu, Y.-J. Natural Interferon α/β-Producing Cells Link Innate and Adaptive Immunity. Journal of Experimental Medicine 192, 219-226, doi:10.1084/jem.192.2.219 (2000).
69. Abbas, A. et al. The activation trajectory of plasmacytoid dendritic cells in vivo during a viral infection. Nature Immunology 21, 983-997, doi:10.1038/s41590-020-0731-4 (2020).
70. Fitzgerald-Bocarsly, P., Dai, J. & Singh, S. Plasmacytoid dendritic cells and type I IFN: 50 years of convergent history. Cytokine & growth factor reviews 19, 3-19, doi:10.1016/j.cytogfr.2007.10.006 (2008).
71. Madera, S. et al. Type I IFN promotes NK cell expansion during viral infection by protecting NK cells against fratricide. The Journal of experimental medicine 213, 225-233, doi:10.1084/jem.20150712 (2016).
72. Nguyen, K. B. et al. Coordinated and Distinct Roles for IFN-αβ, IL-12, and IL-15 Regulation of NK Cell Responses to Viral Infection. The Journal of Immunology 169, 4279-4287, doi:10.4049/jimmunol.169.8.4279 (2002).
73. Margolin, A. A. et al. ARACNE: An Algorithm for the Reconstruction of Gene Regulatory Networks in a Mammalian Cellular Context. BMC Bioinformatics 7, S7, doi:10.1186/1471-2105-7-S1-57 (2006).
74. Subramanian, A. et al. Gene set enrichment analysis: A knowledge-based approach for interpreting genome-wide expression profiles. Proceedings of the National Academy of Sciences 102, 15545-15550, doi:10.1073/pnas.0506580102 (2005).
75. Laustsen, A. et al. Interferon priming is essential for human CD34+ cell-derived plasmacytoid dendritic cell maturation and function. Nature Communications 9, 3525, doi:10.1038/541467-018-05816-y (2018).
76. Gerondakis, S. & Siebenlist, U. Roles of the NF-kappaB pathway in lymphocyte development and function. Cold Spring Harbor perspectives in biology 2, a000182-a000182, doi:10.1101/cshperspect.a000182 (2010).
77. Ben-Sasson, S. Z. et al. IL-1 enhances expansion, effector function, tissue localization, and memory response of antigen-specific CD8 T cells. The Journal of experimental medicine 210, 491-502, doi:10.1084/jem.20122006 (2013).
78. Tabiasco, J. et al. Human Effector CD8<sup>+</sup> T Lymphocytes Express TLR3 as a Functional Coreceptor. The Journal of Immunology 177, 8708-8713, doi:10.4049/jimmunol.177.12.8708 (2006).
79. Tabiasco, J. et al. Human effector CD8+ T lymphocytes express TLR3 as a functional coreceptor. J Immunol 177, 8708-8713, doi:10.4049/jimmunol.177.12.8708 (2006).
80. Suzuki, I. & Fink, P. J. The dual functions of Fas ligand in the regulation of peripheral CD8<sup>+</sup> and CD4<sup>+</sup> T cells. Proceedings of the National Academy of Sciences 97, 1707-1712, doi:10.1073/pnas.97.4.1707 (2000).
81. Araki, K., Youngblood, B. & Ahmed, R. The role of mTOR in memory CD8 T-cell differentiation. Immunol Rev 235, 234-243, doi:10.1111/j.0105-2896.2010.00898.x (2010).
82. Araki, K. et al. mTOR regulates memory CD8 T-cell differentiation. Nature 460, 108-112, doi:10.1038/nature08155 (2009).
83. Dunkelberger, J. R. & Song, W.-C. Complement and its role in innate and adaptive immune responses. Cell Research 20, 34-50, doi:10.1038/cr.2009.139 (2010).
84. Clarke, E. V. & Tenner, A. J. Complement modulation of T cell immune responses during homeostasis and disease. Journal of leukocyte biology 96, 745-756, doi:10.1189/jlb.3MR0214-109R (2014).
85. Arbore, G. et al. Complement receptor CD46 co-stimulates optimal human CD8+ T cell effector function via fatty acid metabolism. Nature Communications 9, 4186, doi:10.1038/s41467-018-06706-z (2018).
86. Adams, N. M., Grassmann, S. & Sun, J. C. Clonal expansion of innate and adaptive lymphocytes. Nature Reviews Immunology 20, 694-707, doi:10.1038/s41577-020-0307-4 (2020).
87. Waldman, A. D., Fritz, J. M. & Lenardo, M. J. A guide to cancer immunotherapy: from T cell basic science to clinical practice. Nature Reviews Immunology 20, 651-668, doi:10.1038/s41577-020-0306-5 (2020).
88. Wu, T. D. et al. Peripheral T cell expansion predicts tumour infiltration and clinical response. Nature 579, 274-278, doi:10.1038/s41586-020-2056-8 (2020).
89. Cui, J.-H. et al. TCR Repertoire as a Novel Indicator for Immune Monitoring and Prognosis Assessment of Patients With Cervical Cancer. Frontiers in Immunology 9, doi:10.3389/fimmu.2018.02729 (2018).
90. Greiff, V. et al. A bioinformatic framework for immune repertoire diversity profiling enables detection of immunological status. Genome medicine 7, 49-49, doi:10.1186/s13073-015-0169-8 (2015).
91. Venturi, V., Kedzierska, K., Turner, S. J., Doherty, P. C. & Davenport, M. P. Methods for comparing the diversity of samples of the T cell receptor repertoire. Journal of Immunological Methods 321, 182-195, doi:https://doi.org/10.1016/j.jim.2007.01.019 (2007).
92. Villanueva, M. T. STINGing systemically. Nature Reviews Drug Discovery 18, 15-15, doi:10.1038/nrd.2018.236 (2019).
93. Barber, G. N. STING: infection, inflammation and cancer. Nature Reviews Immunology 15, 760-770, doi:10.1038/nri3921 (2015).
94. van der Zanden, S. Y., Luimstra, J. J., Neefjes, J., Borst, J. & Ovaa, H. Opportunities for Small Molecules in Cancer Immunotherapy. Trends in Immunology 41, 493-511, doi:10.1016/j.it.2020.04.004 (2020).
95. Berger, G., Marloye, M. & Lawler, S. E. Pharmacological Modulation of the STING Pathway for Cancer Immunotherapy. Trends in Molecular Medicine 25, 412-427, doi:10.1016/j.molmed.2019.02.007 (2019).
96. Karki, R. & Kanneganti, T.-D. Diverging inflammasome signals in tumorigenesis and potential targeting. Nature Reviews Cancer 19, 197-214, doi:10.1038/541568-019-0123-y (2019).
97. Wang, F. et al. Tumour sensitization via the extended intratumoural release of a STING agonist and camptothecin from a self-assembled hydrogel. Nature Biomedical Engineering, doi:10.1038/s41551-020-0597-7 (2020).
98. Pan, B.-S. et al. An orally available non-nucleotide STING agonist with antitumor activity. Science 369, eaba6098, doi:10.1126/science.aba6098 (2020).
99. Chin, E. N. et al. Antitumor activity of a systemic STING-activating non-nucleotide cGAMP mimetic. Science 369, 993-999, doi:10.1126/science.abb4255 (2020).
100. Hetzer, M. W. The nuclear envelope. Cold Spring Harbor perspectives in biology 2, a000539-a000539, doi:10.1101/cshperspect.a000539 (2010).
101. Arnone, J. T., Walters, A. D. & Cohen-Fix, O. The dynamic nature of the nuclear envelope: lessons from closed mitosis. Nucleus (Austin, Tex.) 4, 261-266, doi:10.4161/nucl.25341 (2013).
102. Suresh, S. Biomechanics and biophysics of cancer cells. Acta Materialia 55, 3989-4014, doi:https://doi.org/10.1016/j.actamat.2007.04.022 (2007). 103. Sampson, J. H. et al. Greater chemotherapy-induced lymphopenia enhances tumor-specific immune responses that eliminate EGFRvIII-expressing tumor cells in patients with glioblastoma. Neuro Oncol 13, 324-333, doi:10.1093/neuonc/noq157 (2011).
104. Karachi, A., Dastmalchi, F., Mitchell, D. A. & Rahman, M. Temozolomide for immunomodulation in the treatment of glioblastoma. Neuro-Oncology 20, 1566-1572, doi:10.1093/neuonc/noy072 (2018).
105. Simnica, D. et al. T cell receptor next-generation sequencing reveals cancer-associated repertoire metrics and reconstitution after chemotherapy in patients with hematological and solid tumors. Oncolmmunology 8, e1644110, doi:10.1080/2162402X.2019.1644110 (2019).
106. Grossman, S. A. et al. Iatrogenic immunosuppression in patients with high-grade gliomas treated with radiation and temozolomide: A NABTT CNS Consortium study. Journal of Clinical Oncology 28, 2013-2013, doi:10.1200/jco.2010.28.15_suppl.2013 (2010).
107. Su, Y. B. et al. Selective CD4+ Lymphopenia in Melanoma Patients Treated With Temozolomide: A Toxicity With Therapeutic Implications. Journal of Clinical Oncology 22, 610-616, doi:10.1200/jco.2004.07.060 (2004).
108. Ridola, V. et al. Feasibility study of 21-day-on/7-day-off temozolomide in children with brain tumors. Journal of Neuro-Oncology 103, 147-153, doi:10.1007/s11060-010-0371-x (2011).
109. Lin, A. J. et al. Impact of concurrent versus adjuvant chemotherapy on the severity and duration of lymphopenia in glioma patients treated with radiation therapy. Journal of Neuro-Oncology 136, 403-411, doi:10.1007/s11060-017-2668-5 (2018).
110. Grossman, S. A. et al Immunosuppression in Patients with High-Grade Gliomas Treated with Radiation and Temozolomide. Clinical Cancer Research 17, 5473-5480, doi:10.1158/1078-0432.Ccr-11-0774 (2011).
111. Fadul, C. E. et al Immune modulation effects of concomitant temozolomide and radiation therapy on peripheral blood mononuclear cells in patients with glioblastoma multiforme. Neuro-Oncology 13, 393-400, doi:10.1093/neuonc/noq204 (2011).
112. Ellsworth, S. et al. Sustained CD4+ T cell-driven lymphopenia without a compensatory IL-7/IL-15 response among high-grade glioma patients treated with radiation and temozolomide. Oncolmmunology 3, e27357, doi:10.4161/onci.27357 (2014).
113. Van Meir, E. G. et al. Analysis of the p53 gene and its expression in human glioblastoma cells. Cancer Res 54, 649-652 (1994). 114. Ishii, N. et al. Frequent co-alterations of TP53, p16/CDKN2A, p14ARF, PTEN tumor suppressor genes in human glioma cell lines. Brain Pathol 9, 469-479, doi:10.1111/j.1750-3639.1999.tb00536.x (1999).
115. Flores, C. T. et al. Lin-CCR2+ hematopoietic stem and progenitor cells overcome resistance to PD-1 blockade. Nature Communications 9, 4313, doi:10.1038/s41467-018-06182-5 (2018).
116. Butler, A., Hoffman, P., Smibert, P., Papalexi, E. & Satija, R. Integrating single-cell transcriptomic data across different conditions, technologies, and species. Nature Biotechnology 36, 411-420, doi:10.1038/nbt.4096 (2018).
117. Stuart, T. et al. Comprehensive Integration of Single-Cell Data. Cell 177, 1888-1902.e1821, doi:10.1016/j.cell.2019.05.031 (2019).
118. Becht, E. et al. Dimensionality reduction for visualizing single-cell data using UMAP. Nature Biotechnology 37, 38-44, doi:10.1038/nbt.4314 (2019).
119. Tang, M. et al. Evaluating single-cell cluster stability using the Jaccard similarity index. bioRxiv, 2020.2005.2026.116640, doi:10.1101/2020.05.26.116640 (2020).
120. Phipson, B., Lee, S., Majewski, I. J., Alexander, W. S. & Smyth, G. K. Robust hyperparameter estimation protects against hypervariable genes and improves power to detect differential expression. Ann. Appl. Stat. 10, 946-963, doi:10.1214/16-AOAS920 (2016).
121. Ritchie, M. E. et al. limma powers differential expression analyses for RNA-sequencing and microarray studies. Nucleic Acids Research 43, e47-e47, doi:10.1093/nadgkv007 (2015).
122. Law, C. W., Chen, Y., Shi, W. & Smyth, G. K. voom: precision weights unlock linear model analysis tools for RNA-seq read counts. Genome Biology 15, R29, doi:10.1186/gb-2014-15-2-r29 (2014).
123. Subramanian, A. et al. Gene set enrichment analysis: A knowledge-based approach for interpreting genome-wide expression profiles. Proceedings of the National Academy of Sciences 102, 15545-15550, doi:10.1073/pnas.0506580102 (2005).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the claims listed below, and equivalents thereof.

TABLE S1
related to FIGS. 5-6: Baseline patient characteristics

| | | | MGMT | idh | Daily dex | Daily dex |
|---|---|---|---|---|---|---|
| | | Age | promoter | mutation by | dose at 1^{st} | dose at 2^{nd} |
| Pt ID | Sex | (years) | methylation | IHC or NGS | PBMCs | PBMCs |
| 7 | Male | 70-80 | negative | negative | 0 mg | 0 mg |
| 9 | Female | 50-60 | positive | negative | 4 mg | 2 mg |
| 12 | Male | 60-70 | negative | negative | 0 mg | 0 mg |
| 14 | Male | 70-80 | positive | negative | 0 mg | 2 mg |
| 16 | Female | 40-50 | negative | negative | 2 mg | 0 mg |
| 18 | Male | 50-60 | negative | negative | 0 mg | 0 mg |
| 19 | Male | 60-70 | positive | negative | 4 mg | 4 mg |
| 22 | Male | 30-40 | negative | positive | 0 mg | 0 mg |
| 23 | Male | 60-70 | negative | negative | 0 mg | 0 mg |
| 24 | Male | 30-40 | negative | negative | 0 mg | 3 mg |
| 25 | Male | 60-70 | negative | negative | 2 mg | 4 mg |
| 28 | Male | 60-70 | negative | negative | 0 mg | 2 mg |

TABLE S2
related to FIGS. 5-6:
Viability of cryopreserved PBMCs
(% Live cell)

| Patient | Pre TTF | Post TTF |
|---|---|---|
| P7 | 87 | 95 |
| P9 | 90 | 93 |
| P12 | 92 | 96 |
| P14 | 97 | 96 |
| P16 | 96 | 96 |
| P18 | 94 | 95 |
| P19 | 96 | 95 |
| P22 | 88 | 96 |
| P23 | 89 | 69 |
| P24 | 90 | 90 |
| P25 | 95 | 98 |
| P28 | 96 | 94 |

TABLE S3
related to FIG. 5a-b: Parameters and quality control of single cell RNA-seq of PBMCs in GBM patients

| | P7 | | P9 | | P12 | |
|---|---|---|---|---|---|---|
| Patient | Pre- | Post- | Pre- | Post- | Pre- | Post- |
| TTF | TTF | TTF | TTF | TTF | TTF | TTF |
| Estimated Number of Cells | 8634 | 8213 | 5816 | 5003 | 8940 | 8395 |
| Meand Reads per Cell | 50849 | 47544 | 91050 | 87233 | 46880 | 66568 |
| Median Genes per Cell | 1573 | 1465 | 2042 | 2080 | 1069 | 1102 |
| Number of Reads | 1.1E+08 | 3.9E+08 | 5.3E+08 | 4.4E+08 | 4.2E+08 | 4.3E+08 |
| Valid Barcodes | 96.60% | 96.90% | 97.20% | 92.20% | 98.20% | 98.30% |
| Sequencing Saturation | 68.50% | 69.70% | [symbol] | 78.50% | 82.60% | 87.40% |
| Q30 Bases in Barcode | 93.70% | 93.70% | [symbol] | [symbol] | 93.90% | 93.70% |
| Q30 Bases in RNA Read | 88.20% | 88.40% | [symbol] | 88.70% | 89.30% | 89.00% |
| Q30 Bases in UMI | 92.60% | 92.70% | 92.50% | 92.50% | 93.60% | 93.40% |
| Reads Mapped to Genome | 95.00% | 95.40% | 92.00% | 94.40% | 94.60% | 94.30% |
| Reads Mapped Confidently to Genome | 92.40% | 93.20% | 84.40% | 88.40% | 92.60% | 92.30% |
| Reads Mapped Confidently to Intergenic Regions | 5.50% | 5.20% | 6.70% | 5.80% | 4.80% | 4.90% |
| Reads Mapped Confidently to Intronic Regions | 33.20% | 31.40% | 30.10% | 34.50% | 36.30% | 34.10% |
| Reads Mapped Confidently to Exonic Regions | 53.60% | 56.50% | 47.70% | 48.20% | 51.60% | 52.30% |
| Reads Mapped Confidently to Transcription | 49.60% | 54.70% | [symbol] | 44.00% | 47.70% | 48.50% |
| Read Mapped Antisense to Gene | 1.30% | 1.00% | 1.20% | 1.30% | 1.20% | 1.10% |
| Fraction Reads in Cells | 96.00% | 94.10% | [symbol] | [symbol] | 92.30% | 90.60% |
| Total Genes Detected | 21360 | 20857 | 20075 | 19816 | 20512 | 19805 |
| Median UMI Counts per Cell | 4979 | 4740 | 6510 | 6636 | 2713 | 2911 |
| | P14 | | P16 | | P18 | |
| Patient | Pre- | Post- | Pre- | Post- | Pre- | Post- |
| TTF | TTF | TTF | TTF | TTF | TTF | TTF |
| Estimated Number of Cells | 8966 | 11414 | 12766 | 13031 | 6173 | 7058 |
| Meand Reads per Cell | 45847 | 14677 | 31671 | 30619 | 64866 | 51994 |
| Median Genes per Cell | 1154 | 1015 | 759 | 805 | 1138 | 1019 |
| Number of Reads | 4.1E+08 | 4.0E+08 | 4.0E+08 | 4.0E+08 | 4.0E+08 | 3.7E+08 |
| Valid Barcodes | 98.10% | 98.20% | 98.20% | 98.20% | 98.30% | 98.20% |
| Sequencing Saturation | 79.00% | 78.90% | 78.30% | 79.00% | 87.30% | 89.70% |
| Q30 Bases in Barcode | 93.80% | 93.80% | 93.80% | 93.80% | 93.90% | 93.80% |
| Q30 Bases in RNA Read | 90.30% | 89.80% | 90.30% | 90.40% | 89.10% | 88.90% |
| Q30 Bases in UMI | 93.40% | 93.40% | 93.60% | 93.40% | 93.60% | 93.60% |
| Reads Mapped to Genome | 95.30% | 94.60% | 95.00% | 95.80% | 94.30% | 94.30% |
| Reads Mapped Confidently to Genome | 92.80% | 92.30% | 92.50% | 93.50% | 92.30% | 92.40% |
| Reads Mapped Confidently to Intergenic Regions | 5.20% | 5.70% | 5.20% | 5.40% | 4.80% | 4.30% |
| Reads Mapped Confidently to Intronic Regions | 39.90% | 38.30% | 41.20% | 39.60% | 33.00% | 32.70% |
| Reads Mapped Confidently to Exonic Regions | 47.70% | 48.30% | 46.20% | 48.40% | 54.50% | 55.40% |
| Reads Mapped Confidently to Transcription | 43.80% | 44.30% | 42.70% | 44.60% | 50.60% | 51.40% |
| Read Mapped Antisense to Gene | 1.20% | 1.10% | 0.90% | 1.00% | 1.00% | 1.00% |
| Fraction Reads in Cells | 83.70% | 82.40% | 85.00% | 88.00% | 91.90% | 91.70% |
| Total Genes Detected | 20069 | 20453 | 19193 | 20270 | 19637 | 19631 |
| Median UMI Counts per Cell | 2675 | 2422 | 1837 | 1908 | 2316 | 2587 |
| | P19 | | P22 | | P23 | |
| Patient | Pre- | Post- | Pre- | Post- | Pre- | Post- |
| TTF | TTF | TTF | TTF | TTF | TTF | TTF |
| Estimated Number of Cells | 4816 | 5046 | 5274 | 7171 | 6774 | 5542 |
| Meand Reads per Cell | 90634 | 84888 | 75065 | 47748 | 60568 | 83286 |
| Median Genes per Cell | 1015 | 1067 | 985 | 942 | 1208 | 1132 |
| Number of Reads | 4.4E+08 | 4.3E+08 | 4.0E+08 | 3.4E+08 | 4.1E+08 | 4.6E+08 |
| Valid Barcodes | 98.30% | 98.40% | 98.30% | 98.30% | 98.10% | 98.10% |
| Sequencing Saturation | 92.30% | 90.70% | 89.90% | 86.60% | 81.80% | 86.80% |
| Q30 Bases in Barcode | 93.80% | 93.90% | 94.00% | 93.70% | 94.00% | 94.00% |
| Q30 Bases in RNA Read | 89.20% | 89.20% | 89.70% | 88.50% | 90.30% | 90.20% |
| Q30 Bases in UMI | 93.50% | 93.60% | 93.60% | 93.50% | 93.60% | 93.70% |
| Reads Mapped to Genome | 94.20% | 94.70% | 95.00% | 94.50% | 94.00% | 93.80% |
| Reads Mapped Confidently to Genome | 92.30% | 92.90% | 93.10% | 92.50% | 91.60% | 91.10% |
| Reads Mapped Confidently to Intergenic Regions | 4.70% | 5.50% | 4.90% | 4.50% | 5.90% | 7.10% |
| Reads Mapped Confidently to Intronic Regions | 36.20% | 31.90% | 35.40% | 30.90% | 42.00% | 41.90% |
| Reads Mapped Confidently to Exonic Regions | 51.30% | 55.50% | 52.80% | 57.40% | 43.70% | 42.10% |
| Reads Mapped Confidently to Transcription | 47.90% | 51.90% | 49.10% | 53.40% | 39.60% | 38.00% |
| Read Mapped Antisense to Gene | 1.00% | 0.90% | 1.10% | 0.30% | 1.40% | 1.40% |
| Fraction Reads in Cells | 89.50% | 86.40% | 92.20% | 93.70% | 93.50% | 87.30% |
| Total Genes Detected | 18631 | 19193 | 19370 | 19661 | 20610 | 20326 |
| Median UMI Counts per Cell | 2301 | 2696 | 2574 | 2434 | 3461 | 3101 |
| | P24 | | P25 | | P28 | |
| Patient | Pre- | Post- | Pre- | Post- | Pre- | Post- |
| TTF | TTF | TTF | TTF | TTF | TTF | TTF |
| Estimated Number of Cells | 8105 | 10869 | 11530 | 8003 | 11302 | 6319 |
| Meand Reads per Cell | 56751 | 40230 | 38888 | 53761 | 37767 | 58227 |
| Median Genes per Cell | 1508 | 588 | 1313 | 1389 | 1054 | 1018 |
| Number of Reads | 4.6E+08 | 4.4E+08 | 4.5E+08 | 4.3E+08 | 4.3E+08 | 4.0E+08 |
| Valid Barcodes | 98.10% | 97.90% | 97.50% | 97.90% | 97.90% | 97.60$ |
| Sequencing Saturation | 76.80% | 80.90% | 72.00% | 76.80% | 76.80% | 84.50% |
| Q30 Bases in Barcode | 94.00% | 93.90% | 94.00% | 94.00% | 94.10% | 93.90% |
| Q30 Bases in RNA Read | 90.50% | 90.50% | 90.20% | 88.80% | 90.20% | 90.30% |
| Q30 Bases in UMI | 93.60% | 93.60% | 93.60% | 93.60% | 93.70% | 93.70% |
| Reads Mapped to Genome | 94.30% | 94.30% | 94.50% | 89.30% | 94.40% | 94.10% |
| Reads Mapped Confidently to Genome | 91.80% | 91.50% | 92.40% | 87.30% | 91.70% | 91.60% |
| Reads Mapped Confidently to Intergenic Regions | 5.10% | 5.60% | 5.20% | 5.20% | 6.00% | 6.60% |
| Reads Mapped Confidently to Intronic Regions | 41.40% | 42.70% | 44.00% | 43.80% | 44.70% | 43.80% |
| Reads Mapped Confidently to Exonic Regions | 45.30% | 43.20% | 43.20% | 38.20% | 41.00% | 41.20% |
| Reads Mapped Confidently to Transcription | 41.30% | 39.10% | 39.00% | 34.30% | 37.00% | 37.10% |
| Read Mapped Antisense to Gene | 1.40% | 1.50% | 1.70% | 1.60% | 1.50% | 1.60% |
| Fraction Reads in Cells | 93.70% | 91.60% | 94.10% | 96.00% | 93.20% | 89.40% |
| Total Genes Detected | 21060 | 20421 | 21330 | 20780 | 21238 | 20291 |
| Median UMI Counts per Cell | 4065 | 1994 | 2971 | 3186 | 2435 | 2317 |

[symbol]

indicates data missing or illegible when filed

TABLE S4
related to FIGS. 5-6: Parameters and quality control of bulk RNA-seq of enriched peripheral T lymphocytes in GBM patients

| | P7 | | P9 | | P12 | |
|---|---|---|---|---|---|---|
| Patient | Pre- | Post- | Pre- | Post- | Pre- | Post- |
| TTF | TTF | TTF | TTF | TTF | TTF | TTF |
| Mapping speed, Million of reads per hour | 8.60 | 4.76 | 4.71 | 8.56 | 8.42 | 8.28 |
| Number of input reads | 9.E+07 | 7.E+07 | 7.E+07 | 7.E+07 | 9.E+07 | 6.E+07 |
| Average input read length | 295 | 296 | 296 | 296 | 296 | 296 |

UNIQUE READS:

| | | | | | | |
|---|---|---|---|---|---|---|
| Uniquiely mapped reads number | 8.3E+07 | 7.2E+07 | 6.7E+07 | 6.7E+07 | 8.1E+07 | 5.4E+07 |
| Unququiely mapped reads % | 93.34% | 95.81% | 96.04% | 96.16% | 91.68% | 91.93% |
| Average mapped length | 291.75 | 292.43 | 292.18 | 292.82 | 291.40 | 291.60 |
| Number of splices: Total | 7.4E+07 | 6.7E+07 | 6.2E+07 | 6.7E+07 | 8.0E+07 | 5.7E+07 |
| Number of splices: Annotated (sjdb) | 7.3E+07 | 6.6E+07 | 6.1E+07 | 6.6E+07 | 7.9E+07 | 5.6E+07 |
| Number of splices: GT/AG | 7.3E+07 | 6.8E+07 | 6.1E+07 | 6.6E+07 | 8.0E+07 | 5.7E+07 |
| Number of splices: GC/AG | 5.0E+05 | 4.5E+05 | 4.2E+05 | 4.5E+05 | 4.9E+05 | 3.8E+05 |
| Number of splices: AT/AC | 5.7E+04 | 5.5E+04 | 5.3E+04 | 5.6E+04 | 6.2E+04 | 4.3E+04 |
| Number of splices: Non canonical | 6.9E+04 | 5.8E+04 | 5.7E+04 | 6.3E+04 | 8.2E+04 | 6.8E+04 |
| Mismatch rate per base % | 0.27% | 0.25% | 0.26% | 0.25% | 0.26% | 0.25% |
| Deletion rate per base | 0.01% | 0.01% | 0.01% | 0.01% | 0.01% | 0.01% |
| Deletion average length | 1.86 | 1.89 | 1.92 | 1.92 | 1.84 | 1.88 |
| Insertion rate per base | 0.01% | 0.01% | 0.01% | 0.01% | 0.01% | 0.01% |
| Insertion average length | 1.70 | 1.80 | 1.78 | 1.78 | 1.60 | 1.63 |

MULTI-MAPPING READS:

| | | | | | | |
|---|---|---|---|---|---|---|
| Number of read mapped to multiple loci | 5.9E+06 | 3.1E+06 | 2.7E+06 | 2.6E+06 | 6.5E+06 | 8.7E+06 |
| % of reads mapped to mulitple loci | 6.50% | 4.13% | 3.92% | 3.80% | 7.34% | 8.03% |
| Number of reads mapped to too many loci | 15605 | 12488 | 10755 | 7850 | 12358 | 6867 |
| % of reads mapped to too many loci | 0.02% | 0.02% | 0.02% | 0.01% | 0.01% | 0.01% |

UNMAPPED READS:

| | | | | | | |
|---|---|---|---|---|---|---|
| % of reads unmapped: too many mismatches | 0.00% | 0.00% | 0.00% | 0.00% | 0.00% | 0.00% |
| % of reads unmapped: too short | 0.00% | 0.00% | 0.00% | 0.00% | 0.00% | 0.00% |
| % of reads unmapped: other | 0.04% | 0.04% | 0.03% | 0.02% | 0.02% | 0.03% |

CHIMERIC READS:

| | | | | | | |
|---|---|---|---|---|---|---|
| Number of chimeric reads | 1.8E+06 | 1.6E+06 | 1.4E+06 | 1.3E+06 | 1.8E+06 | 1.5E+06 |
| % of chimeric reads | 1.99% | 2.11% | 1.02% | 1.93% | 1.68% | 2.53% |

| | P14 | | P16 | | P18 | |
|---|---|---|---|---|---|---|
| Patient | Pre- | Post- | Pre- | Post- | Pre- | Post- |
| TTF | TTF | TTF | TTF | TTF | TTF | TTF |
| Mapping speed, Million of reads per hour | 7.94 | 7.35 | 8.41 | 6.19 | 7.58 | 3.69 |
| Number of input reads | 1.E+08 | 8.E+07 | 7.E+07 | 8.E+07 | 7.E+07 | 8.E+07 |
| Average input read length | 296 | 295 | 296 | 295 | 296 | 296 |

UNIQUE READS:

| | | | | | | |
|---|---|---|---|---|---|---|
| Uniquiely mapped reads number | 9.3E+07 | 6.5E+07 | 7.0E+07 | 6.9E+07 | 7.1E+07 | 5.6E+07 |
| Unququiely mapped reads % | 94.96% | 94.10% | 95.65% | 88.93% | 94.86% | 95.19% |
| Average mapped length | 290.84 | 287.15 | 291.92 | 276.17 | 289.64 | 290.88 |
| Number of splices: Total | 9.7E+07 | 5.1E+07 | 6.7E+07 | 6.0E+07 | 7.1E+07 | 4.7E+07 |
| Number of splices: Annotated (sjdb) | 9.5E+07 | 5.0E+07 | 6.6E+07 | 5.9E+07 | 7.0E+07 | 4.6E+07 |
| Number of splices: GT/AG | 9.6E+07 | 5.1E+07 | 8.7E+07 | 5.9E+07 | 7.0E+07 | 4.6E+07 |
| Number of splices: GC/AG | 6.7E+05 | [symbol] | 4.6E+05 | 1.1E+05 | 4.7E+05 | [symbol] |
| Number of splices: AT/AC | 7.3E+04 | 3.9E+04 | 5.0E+04 | 1.4E+04 | 5.8E+04 | [symbol] |
| Number of splices: Non canonical | 9.7E+04 | 5.5E+04 | 6.5E+04 | 5.7E+04 | 7.3E+04 | 5.0E+04 |
| Mismatch rate per base % | 0.23% | 0.28% | 0.25% | 0.39% | 0.28% | 0.28% |
| Deletion rate per base | 0.01% | 0.01% | 0.01% | 0.05% | 0.01% | 0.01% |
| Deletion average length | 1.81 | 1.85 | 1.88 | 1.45 | 1.95 | 1.99 |
| Insertion rate per base | 0.01% | 0.01% | 0.01% | 0.01% | 0.01% | 0.01% |
| Insertion average length | 1.73 | 1.75 | 1.76 | 1.85 | 1.72 | 1.71 |

MULTI-MAPPING READS:

| | | | | | | |
|---|---|---|---|---|---|---|
| Number of read mapped to multiple loci | 4.9E+06 | 3.4E+06 | 3.1E+06 | 8.6E+06 | 3.8E+06 | 2.8E+06 |
| % of reads mapped to mulitple loci | 4.99% | 5.85% | 4.30% | 11.01% | 5.09% | 4.75% |
| Number of reads mapped to too many loci | 14113 | 15464 | 10342 | 36498 | 16980 | 15688 |
| % of reads mapped to too many loci | 0.01% | 0.03% | 0.01% | 0.05% | 0.02% | 0.02% |

UNMAPPED READS:

| | | | | | | |
|---|---|---|---|---|---|---|
| % of reads unmapped: too many mismatches | 0.00% | 0.00% | 0.00% | 0.00% | 0.00% | 0.00% |
| % of reads unmapped: too short | 0.00% | 0.00% | 0.00% | 0.00% | 0.00% | 0.00% |
| % of reads unmapped: other | 0.04% | 0.02% | 0.03% | 0.02% | 0.03% | 0.04% |

CHIMERIC READS:

| | | | | | | |
|---|---|---|---|---|---|---|
| Number of chimeric reads | 1.7E+06 | 1.5E+06 | 1.5E+06 | 4.9E+05 | 1.5E+06 | 1.0E+06 |
| % of chimeric reads | 1.69% | 2.51% | 2.00% | 0.63% | 1.98% | 1.75% |

| | P19 | | P22 | | P23 | |
|---|---|---|---|---|---|---|
| Patient | Pre- | Post- | Pre- | Post- | Pre- | Post- |
| TTF | TTF | TTF | TTF | TTF | TTF | TTF |
| Mapping speed, Million of reads per hour | 8.74 | 5.16 | 8.19 | 7.76 | 2.76 | 5.21 |
| Number of input reads | 8.E+07 | 8.E+07 | 8.E+07 | 7.E+07 | 8.E+07 | 9.E+07 |
| Average input read length | 296 | 295 | 295 | 296 | 296 | 295 |

UNIQUE READS:

| | | | | | | |
|---|---|---|---|---|---|---|
| Uniquiely mapped reads number | 7.4E+07 | 5.9E+0 | 7.8E+07 | 7.0E+07 | 7.5E+07 | 9.0E+07 |
| Unququiely mapped reads % | 96.18% | 96.71% | 96.18% | 95.89% | 95.52% | 86.28% |
| Average mapped length | 291.96 | 291.16 | 292.09 | 292.29 | 290.34 | 292.74 |
| Number of splices: Total | 6.8E+07 | 5.1E+07 | 8.5E+07 | 8.4E+07 | 7.9E+07 | 8.5E+07 |
| Number of splices: Annotated (sjdb) | 6.7E+07 | 9.0E+07 | 8.4E+07 | 8.3E+07 | 7.8E+07 | 8.3E+07 |
| Number of splices: GT/AG | 6.7E+07 | 9.1E+07 | 8.4E+07 | 8.3E+07 | 7.9E+07 | 8.4E+07 |
| Number of splices: GC/AG | 4.7E+05 | 3.5E+05 | 5.5E+05 | 5.2E+05 | 5.0E+05 | 5.6E+05 |
| Number of splices: AT/AC | 6.0E+04 | 4.3E+04 | 7.5E+04 | 6.2E+04 | 5.7E+04 | 6.6E+04 |
| Number of splices: Non canonical | 7.4E+04 | 5.5E+04 | 8.3E+04 | 2.6E+04 | 7.2E+04 | 2.9E+04 |
| Mismatch rate per base % | 0.27% | 0.39% | 0.25% | 0.25% | 0.26% | 0.25% |
| Deletion rate per base | 0.01% | 0.01% | 0.01% | 0.01% | 0.01% | 0.01% |
| Deletion average length | 1.95 | 1.93 | 1.83 | 1.92 | 1.63 | 1.84 |
| Insertion rate per base | 0.01% | 0.01% | 0.01% | 0.01% | 0.01% | 0.01% |
| Insertion average length | 1.79 | 1.78 | 1.74 | 1.81 | 1.79 | 1.82 |

MULTI-MAPPING READS:

| | | | | | | |
|---|---|---|---|---|---|---|
| Number of read mapped to multiple loci | 2.9E+06 | 2.3E+06 | 3.1E+06 | 3.0E+06 | 3.5E+06 | 3.4E+06 |
| % of reads mapped to mulitple loci | 3.77% | 3.74% | 3.79% | 4.08% | 4.43% | 3.69% |
| Number of reads mapped to too many loci | 11278 | 9285 | 7437 | 6652 | 19039 | 14596 |
| % of reads mapped to too many loci | 0.01% | 0.02% | 0.01% | 0.01% | 0.02% | 0.02% |

UNMAPPED READS:

| | | | | | | |
|---|---|---|---|---|---|---|
| % of reads unmapped: too many mismatches | 0.00% | 0.00% | 0.00% | 0.00% | 0.00% | 0.00% |
| % of reads unmapped: too short | 0.00% | 0.00% | 0.00% | 0.00% | 0.00% | 0.00% |
| % of reads unmapped: other | 0.03% | 0.03% | 0.02% | 0.03% | 0.04% | 0.04% |

CHIMERIC READS:

| | | | | | | |
|---|---|---|---|---|---|---|
| Number of chimeric reads | 1.6E+06 | 1.4E+06 | 1.9E+06 | 1.3E+06 | 1.3E+06 | 1.6E+06 |
| % of chimeric reads | 2.11% | 2.31% | 2.32% | 1.74% | 1.63% | 1.74% |

| | P24 | | P25 | | P28 | |
|---|---|---|---|---|---|---|
| Patient | Pre- | Post- | Pre- | Post- | Pre- | Post- |
| TTF | TTF | TTF | TTF | TTF | TTF | TTF |
| Mapping speed, Million of reads per hour | 4.85 | 7.95 | 6.02 | 8.30 | 3.10 | 8.50 |
| Number of input reads | 4.E+08 | 4.E+08 | 9.E+07 | 9.E+07 | 9.E+07 | 1.E+08 |
| Average input read length | 295 | 296 | 296 | 296 | 296 | 296 |

UNIQUE READS:

| | | | | | | |
|---|---|---|---|---|---|---|
| Uniquiely mapped reads number | 1.1E+08 | 9.5E+07 | 9.0E+07 | 9.1E+07 | 8.6E+07 | 9.3E+07 |
| Unququiely mapped reads % | 95.10% | 95.88% | 96.54% | 86.49% | 95.03% | 96.14% |
| Average mapped length | 291.82 | 292.50 | 293.44 | 293.85 | 292.62 | 292.90 |
| Number of splices: Total | 1.1E+08 | 1.0E+08 | 8.3E+07 | 9.3E+07 | 9.2E+07 | 9.4E+07 |
| Number of splices: Annotated (sjdb) | 1.1E+08 | 1.0E+08 | 8.2E+07 | 9.2E+07 | 9.1E+07 | 9.3E+07 |
| Number of splices: GT/AG | 1.1E+08 | 1.0E+08 | 8.2E+07 | 9.3E+07 | 9.1E+07 | 9.3E+07 |
| Number of splices: GC/AG | 7.4E+05 | 6.7E+05 | 5.6E+05 | 6.3E+05 | 5.9E+05 | 8.2E+05 |
| Number of splices: AT/AC | 9.8E+04 | 9.0E+04 | 7.1E+04 | 7.0E+04 | 8.1E+04 | 8.2E+04 |
| Number of splices: Non canonical | 1.1E+05 | 1.0E+05 | 7.1E+04 | 8.3E+04 | 1.1E+05 | 1.0E+05 |
| Mismatch rate per base % | 0.28% | 0.25% | 0.24% | 0.22% | 0.27% | 0.25% |
| Deletion rate per base | 0.01% | 0.01% | 0.01% | 0.01% | 0.01% | 0.01% |
| Deletion average length | 1.89 | 1.92 | 1.93 | 1.90 | 2.03 | 2.03 |
| Insertion rate per base | 0.01% | 0.01% | 0.01% | 0.01% | 0.01% | 0.01% |
| Insertion average length | 1.82 | 1.73 | 1.70 | 1.71 | 1.67 | 1.67 |

MULTI-MAPPING READS:

| | | | | | | |
|---|---|---|---|---|---|---|
| Number of read mapped to multiple loci | 4.2E+06 | 4.1E+06 | 3.2E+06 | 3.3E+06 | 3.5E+06 | 3.7E+06 |
| % of reads mapped to mulitple loci | 3.87% | 4.08% | 3.42% | 3.52% | 3.93% | 3.82% |
| Number of reads mapped to too many loci | 11546 | 10260 | 12102 | 11215 | 8365 | 10525 |
| % of reads mapped to too many loci | 0.01% | 0.01% | 0.01% | 0.01% | 0.01% | 0.01% |

UNMAPPED READS:

| | | | | | | |
|---|---|---|---|---|---|---|
| % of reads unmapped: too many mismatches | 0.00% | 0.00% | 0.00% | 0.00% | 0.00% | 0.00% |
| % of reads unmapped: too short | 0.00% | 0.00% | 0.00% | 0.00% | 0.00% | 0.00% |
| % of reads unmapped: other | 0.02% | 0.02% | 0.03% | 0.03% | 0.03% | 0.03% |

CHIMERIC READS:

| | | | | | | |
|---|---|---|---|---|---|---|
| Number of chimeric reads | 2.4E+06 | 1.8E+06 | 1.3E+06 | 1.2E+06 | 1.8E+06 | 2.3E+06 |
| % of chimeric reads | 2.21% | 1.85% | 1.35% | 1.27% | 1.99% | 2.43% |

[symbol]

indicates data missing or illegible when filed

TABLE 5
supporting FIG. 5i: Top T1IRG and T2IRG pathways upregulated after
TTFields in pDCs (C31)

| PATHWAY NAME PER GENE ONTOLOGY (GO) | SIZE | NES | NOM p-val |
|---|---|---|---|
| GO_REGULATION_OF_RESPONSE_TO_INTERFERON_GAMMA | 21.00 | 1.86 | 0.02 |
| GO_CELLULAR_RESPONSE_TO_INTERFERON_ALPHA | 8.00 | 1.83 | 0.02 |
| GO_RESPONSE_TO_TYPE_I_INTERFERON | 73.00 | 1.81 | 0.01 |
| GO_RESPONSE_TO_INTERFERON_GAMMA | 139.00 | 1.75 | 0.02 |
| GO_INTERFERON_GAMMA_MEDIATED_SIGNALING_PATHWAY | 73.00 | 1.40 | 0.11 |
| GO_POSITIVE_REGULATION_OF_TYPE_I_INTERFERON_ | 10.00 | 1.32 | 0.16 |
| MEDIATED_SIGNALING_PATHWAY | | | |
| GO_NEGATIVE_REGULATION_OF_RESPONSE_ | 5.00 | 1.28 | 0.18 |
| TO_INTERFERON_GAMMA | | | |
| GO_RESPONSE_TO_INTERFERON_ALPHA | 17.00 | 1.28 | 0.18 |
| GO_REGULATION_OF_TYPE_I_INTERFERON_ | 27.00 | 1.20 | 0.25 |
| MEDIATED_SIGNALING_PATHWAY | | | |
| GO_TYPE_I_INTERFERON_PRODUCTION | 103.00 | 1.00 | 0.42 |
| GO_NEGATIVE_REGULATION_OF_TYPE_I_INTERFERON_ | 10.00 | 0.94 | 0.51 |
| MEDIATED_SIGNALING_PATHWAY | | | |

TABLE S6
supporting FIG. 6a: Simpson diversity index of
TCRα/β V(D)J in peripheral T cells

| | TCRα | | | TCRβ | | |
|---|---|---|---|---|---|---|
| | Simpson Diversity Index | | | Simpson Diversity Index | | |
| Patient | Pre TTF | Post TTF | logFC | Pre TTF | Post TTF | logFC |
| P7 | 371.6 | 323.7 | −0.2 | 774.0 | 645.0 | −0.26 |
| P9 | 316.3 | 341.8 | 0.11 | 398.0 | 468.0 | 0.51 |
| P12 | 19.7 | 179.5 | 3.19 | 23.0 | 108.0 | 2.22 |
| P14 | 86.9 | 36.4 | −1.25 | 155.0 | 107.0 | −0.54 |
| P16 | 268.6 | 21.4 | −3.65 | 443.0 | 59.0 | −2.92 |
| P18 | 255.7 | 88.6 | −1.53 | 381.0 | 138.0 | −1.47 |
| P19 | 37.0 | 14.5 | −1.35 | 95.0 | 20.0 | −2.24 |
| P22 | 174.8 | 157.4 | −0.15 | 81.0 | 139.0 | 0.77 |
| P23 | 15.8 | 8.9 | −0.83 | 16.0 | 7.0 | −1.27 |
| P24 | 132.8 | 166.6 | 0.33 | 217.0 | 174.0 | −0.32 |
| P25 | 380.4 | 279.4 | −0.45 | 535.0 | 504.0 | −0.09 |
| P28 | 94.4 | 61.2 | −0.63 | 54.0 | 46.0 | −0.23 |

1. A method, comprising:
(a) determining in immune cells of a subject a first expression level of the following biomarker(s):
cytokines and cytotoxic genes,
immune cell functional regulators,
naïve immune cell markers,
regulatory T cell factors, or
immune inhibitory receptors, or
combinations thereof;
(b) applying alternating electric fields to tumor cells of the subject at a frequency between 50 kHz-1 MHz after step (a) and prior to step (c); and
(c) determining in immune cells of the subject a second expression level of the biomarker(s) of step (a).

2. The method of claim 1, wherein the frequency is between 100 kHz and 500 kHz.

3. The method of claim 1, wherein step (a) comprises determining a first expression level of cytokines and cytotoxic genes.

4. The method of claim 1, wherein step (a) comprises determining a first expression level of immune cell functional regulators.

5. The method of claim 1, wherein step (a) comprises determining a first expression level of cytokines and cytotoxic genes, and determining a first expression level of immune cell functional regulators.

6. The method of claim 4, wherein the immune cell functional regulators are T cell functional regulators.

7. The method of claim 1, wherein step (a) comprises determining a first expression level of:
cytokines and cytotoxic genes,
immune cell functional regulators,
naïve immune cell markers,
regulatory T cell factors, and
immune inhibitory receptors.

8. The method of claim 1, wherein
biomarker expression level is determined by nucleic acid expression or by expression of a corresponding protein.

9. The method of claim 1, further comprising treating the subject with a checkpoint inhibitor if:
(i) the first expression level of at least 50% of the cytokines and cytotoxic genes is lower than the second expression level of cytokines and cytotoxic genes,
(ii) the first expression level of at least 50% of the immune cell functional regulators is lower than the second expression level of immune cell functional regulators,
(iii) the first expression level of at least 50% of the naïve immune cell markers is greater than the second expression level of naïve immune cell markers,
(iv) the first expression level of at least 50% of the regulatory T cell factors is greater than the second expression level of regulatory T cell factors, or
(v) the first expression level of at least 50% of the immune inhibitory receptors is either greater than or unchanged compared to the second expression level of immune inhibitory receptors.

10. The method of claim 9, further comprising treating the subject with a checkpoint inhibitor if the first expression level of at least 50% of the cytokines and cytotoxic genes is lower than the second expression level of cytokines and cytotoxic genes.

11. The method of claim 9, further comprising treating the subject with a checkpoint inhibitor if the first expression level of at least 50% of the immune cell functional regulators is lower than the second expression level of immune cell functional regulators.

12. The method of claim 9, further comprising treating the subject with a checkpoint inhibitor if:
(i) the first expression level of at least 50% of the cytokines and cytotoxic genes is lower than the second expression level of cytokines and cytotoxic genes, and
(ii) the first expression level of at least 50% of the immune cell functional regulators is lower than the second expression level of immune cell functional regulators.

13. The method of claim 1, wherein the immune cell functional regulators are T cell functional regulators or the naïve immune cell markers are naïve T cell markers.

14. The method of claim 1, wherein the nucleic acids expressing cytokines and cytotoxic gene are selected from the group consisting of GZMB, GZMH, GZMK, GNLY, PRF1, INFG, NKG7, CX3CR1, CCL3, and CCL4, and combinations thereof.

15. The method of claim 1, wherein the nucleic acids expressing immune cell functional regulators are selected from the group consisting of ZEB2, ZHF683, HOPX, TBX21, ID2, TOX, GF11, EOMES, and HMGB3, and combinations thereof.

16. (canceled)

17. (canceled)

18. (canceled)

19. (canceled)

20. The method of claim 9, wherein the checkpoint inhibitor is selected from the group consisting of ipilimumab, pembrolizumab, nivolumab, cemilimab, atezolimumab, avelumab, durvalumab, IDO1 inhibitors, TIGIT inhibitors, LAG-3 inhibitors, TIM-3 inhibitors, VISTA inhibitors, and B7-H3 inhibitors.

21. The method of claim 1, wherein the tumor cells are selected from the group consisting of brain cells, blood cells, breast cells, pancreatic cells, ovarian cells, lung cells, and mesenchymal cells.

22. The method of claim 21, wherein the tumor cells are brain cells.

23. The method of claim 21, wherein the tumor cells are cancer cells.

24. A kit comprising nucleic acids for detecting expression of cytokines and cytotoxic genes, nucleic acids expressing T cell functional regulators, nucleic acids expressing naïve T cell markers, nucleic acids expressing regulatory T cell factors, and nucleic acids expressing immune inhibitory receptors.

25. The kit of claim 24, wherein the nucleic acids expressing cytokines and cytotoxic genes are selected from the group consisting of GZMB, GZMH, GZMK, GNLY, PRF1, INFG, NKG7, CX3CR1, CCL3, and CCL4, and combinations thereof, and the nucleic acids expressing immune cell functional regulators are selected from the group consisting of ZEB2, ZHF683, HOPX, TBX21, ID2, TOX, GF11, EOMES, and HMGB3, and combinations thereof.

26. (canceled)

27. (canceled)

28. (canceled)

29. (canceled)

30. (canceled)


SEQUENCE LISTING (47 sequences)

SEQ ID NO: 1 - DNA, 19 bp, Artificial Sequence
  Description of Artificial Sequence Synthetic primer
ggtggacaaatgcgacgaa

SEQ ID NO: 2 - DNA, 18 bp, Artificial Sequence
  Description of Artificial Sequence Synthetic primer
tgctgcggcccttgttat

SEQ ID NO: 3 - DNA, 20 bp, Artificial Sequence
  Description of Artificial Sequence Synthetic primer
aagtgctgccgtcattttct

SEQ ID NO: 4 - DNA, 20 bp, Artificial Sequence
  Description of Artificial Sequence Synthetic primer
cctatggccctcattctcac

SEQ ID NO: 5 - DNA, 20 bp, Artificial Sequence
  Description of Artificial Sequence Synthetic primer
gccagcggctgtatattctc

SEQ ID NO: 6 - DNA, 20 bp, Artificial Sequence
  Description of Artificial Sequence Synthetic primer
gctgtaaacccgatccttga

SEQ ID NO: 7 - DNA, 19 bp, Artificial Sequence
  Description of Artificial Sequence Synthetic primer
gactccatcttggctgtga

SEQ ID NO: 8 - DNA, 21 bp, Artificial Sequence
  Description of Artificial Sequence Synthetic primer
tgatttctgctctgacaacct

SEQ ID NO: 9 - DNA, 24 bp, Artificial Sequence
  Description of Artificial Sequence Synthetic primer
gaatgggaggcttgaatactgcct

SEQ ID NO: 10 - DNA, 25 bp, Artificial Sequence
  Description of Artificial Sequence Synthetic primer
tagcaaagatgttctggagcatctc

SEQ ID NO: 11 - DNA, 20 bp, Artificial Sequence
  Description of Artificial Sequence Synthetic primer
ggcatggactgtggtcatga

SEQ ID NO: 12 - DNA, 17 bp, Artificial Sequence
  Description of Artificial Sequence Synthetic primer
accaccatggagaaggc

SEQ ID NO: 13 - DNA, 21 bp, Artificial Sequence
  Description of Artificial Sequence Synthetic primer
tgtaagctatggcccactcca

SEQ ID NO: 14 - DNA, 23 bp, Artificial Sequence
  Description of Artificial Sequence Synthetic primer
agagacacagattgatccatgca

SEQ ID NO: 15 - DNA, 20 bp, Artificial Sequence
  Description of Artificial Sequence Synthetic primer
ctctctcctttcagggccaa

SEQ ID NO: 16 - DNA, 20 bp, Artificial Sequence
  Description of Artificial Sequence Synthetic primer
gagaggcctggctcaacaaa

SEQ ID NO: 17 - DNA, 20 bp, Artificial Sequence
  Description of Artificial Sequence Synthetic primer
caccgggaacgaaagagaag

SEQ ID NO: 18 - DNA, 20 bp, Artificial Sequence
  Description of Artificial Sequence Synthetic primer
tcatagctgggctcctggag

SEQ ID NO: 19 - DNA, 20 bp, Artificial Sequence
  Description of Artificial Sequence Synthetic primer
acatgacttccaagctggcc

SEQ ID NO: 20 - DNA, 20 bp, Artificial Sequence
  Description of Artificial Sequence Synthetic primer
cagaaatcaggaaggctgcc

SEQ ID NO: 21 - DNA, 20 bp, Artificial Sequence
  Description of Artificial Sequence Synthetic primer
gctgcaccaaaagtctctcc

SEQ ID NO: 22 - DNA, 20 bp, Artificial Sequence
  Description of Artificial Sequence Synthetic primer
acatctcctgcttgccttct

SEQ ID NO: 23 - DNA, 20 bp, Artificial Sequence
  Description of Artificial Sequence Synthetic primer
tgaagtggaccctgaaaacc

SEQ ID NO: 24 - DNA, 20 bp, Artificial Sequence
  Description of Artificial Sequence Synthetic primer
taaagccatccaggcgatag

SEQ ID NO: 25 - DNA, 20 bp, Artificial Sequence
  Description of Artificial Sequence Synthetic primer
gggaaggaatgggaatcagt

SEQ ID NO: 26 - DNA, 20 bp, Artificial Sequence
  Description of Artificial Sequence Synthetic primer
cccacagccactctggttat

SEQ ID NO: 27 - DNA, 20 bp, Artificial Sequence
  Description of Artificial Sequence Synthetic primer
gatgagcaactggtgtgtcg

SEQ ID NO: 28 - DNA, 20 bp, Artificial Sequence
  Description of Artificial Sequence Synthetic primer
actgacggtggccataaaac

SEQ ID NO: 29 - DNA, 20 bp, Artificial Sequence
  Description of Artificial Sequence Synthetic primer
catggaggtcaccagttcct

SEQ ID NO: 30 - DNA, 20 bp, Artificial Sequence
  Description of Artificial Sequence Synthetic primer
tttgttttgcttgggtttcc

SEQ ID NO: 31 - DNA, 20 bp, Artificial Sequence
  Description of Artificial Sequence Synthetic primer
ccctatggagatgacggaga

SEQ ID NO: 32 - DNA, 20 bp, Artificial Sequence
  Description of Artificial Sequence Synthetic primer
ctgtctgctggtggagttca

SEQ ID NO: 33 - DNA, 20 bp, Artificial Sequence
  Description of Artificial Sequence Synthetic primer
ccggagaggagacttcacag

SEQ ID NO: 34 - DNA, 20 bp, Artificial Sequence
  Description of Artificial Sequence Synthetic primer
tccacgatttcccagagaac

SEQ ID NO: 35 - DNA, 20 bp, Artificial Sequence
  Description of Artificial Sequence Synthetic primer
aagaagcagattgcccagaa

SEQ ID NO: 36 - DNA, 19 bp, Artificial Sequence
  Description of Artificial Sequence Synthetic primer
cgctgcagttctgtaccac

SEQ ID NO: 37 - DNA, 20 bp, Artificial Sequence
  Description of Artificial Sequence Synthetic primer
gcccagatctacctggacaa

SEQ ID NO: 38 - DNA, 20 bp, Artificial Sequence
  Description of Artificial Sequence Synthetic primer
cctcacagtccatctcagca

SEQ ID NO: 39 - DNA, 20 bp, Artificial Sequence
  Description of Artificial Sequence Synthetic primer
tgtgcaggcactatgaggag

SEQ ID NO: 40 - DNA, 20 bp, Artificial Sequence
  Description of Artificial Sequence Synthetic primer
actctggtccccaatgacag

SEQ ID NO: 41 - DNA, 20 bp, Artificial Sequence
  Description of Artificial Sequence Synthetic primer
ggggtctgacgaaagcagta

SEQ ID NO: 42 - DNA, 20 bp, Artificial Sequence
  Description of Artificial Sequence Synthetic primer
cccattgaatcacacagcat

SEQ ID NO: 43 - DNA, 20 bp, Artificial Sequence
  Description of Artificial Sequence Synthetic primer
gtttgccatgtcacaggatg

SEQ ID NO: 44 - DNA, 20 bp, Artificial Sequence
  Description of Artificial Sequence Synthetic primer
caatgaggcggcagttattt

SEQ ID NO: 45 - DNA, 20 bp, Artificial Sequence
  Description of Artificial Sequence Synthetic primer
ggagcgagaccccactaaca

SEQ ID NO: 46 - DNA, 20 bp, Artificial Sequence
  Description of Artificial Sequence Synthetic primer
acatactcagcaccggcctc

SEQ ID NO: 47 - protein, 4 aa, Artificial Sequence
  Description of Artificial Sequence Synthetic peptide
YVAD